(19) United States
(12) Patent Application Publication
(10) Pub. No.: US 2014/0303167 A1
(43) Pub. Date: Oct. 9, 2014

(54) CDK9 INHIBITORS IN THE TREATMENT OF MIDLINE CARCINOMA

(71) Applicants: Axel Choidas, Herdecke (DE); Bert Klebl, Dortmund (DE); Peter Habenberger, Dortmund (DE); Jan Eickhoff, Herdecke (DE); Roman Thomas, Bornheim (DE); Johannes Heuckmann, Koln (DE)

(72) Inventors: Axel Choidas, Herdecke (DE); Bert Klebl, Dortmund (DE); Peter Habenberger, Dortmund (DE); Jan Eickhoff, Herdecke (DE); Roman Thomas, Bornheim (DE); Johannes Heuckmann, Koln (DE)

(73) Assignee: LEAD DISCOVERY CENTER GMBH, Dortmund (DE)

(21) Appl. No.: 14/240,329

(22) PCT Filed: Aug. 22, 2012
(86) PCT No.: PCT/EP2012/066336

§ 371 Date: May 16, 2014

(30) Foreign Application Priority Data
Aug. 22, 2011 (EP) 11178328.8

(51) Int. Cl. C07D 471/04; C07D 405/10; C07D 401/12; C07D 403/04; C07D 403/12; C07C 53/18; C07D 213/40; C07D 251/16; C07D 401/04

(52) U.S. Cl. CPC C07D 471/04; C07D 251/16; C07D 405/10; C07D 401/12; C07D 401/04; C07D 403/12; C07C 53/18; C07D 213/40; C07D 403/04
USPC 514/236.2; 544/211; 514/245; 544/212; 544/113; 546/312; 514/352; 544/209

(57) ABSTRACT

The present invention relates to a CDK9 inhibitor, especially a selective CDK9 inhibitor, for use in treating, ameliorating and/or preventing midline carcinoma. Also corresponding methods for treating, preventing or ameliorating midline carcinoma are subject of the present invention. Preferably, NUT midline carcinoma is treated with the CDK9 inhibitors in accordance with the present invention.

### PRIORITY

This U.S. Non-provisional application is being filed as a 371 and claims priority under 35 U.S.C. 121 to International Application No. PCT/EP2012/066336 filed Aug. 22, 2012 which claims benefit to EP Application No. 11178328.8 filed Aug. 22, 2011, which are incorporated herein in their entirety for all purposes.

### FIELD

The present invention relates to a CDK9 inhibitor, especially a selective CDK9 inhibitor, for use in treating, ameliorating and/or preventing midline carcinoma. Also corresponding methods for treating, preventing or ameliorating midline carcinoma are subject of the present invention. Preferably, NUT midline carcinoma is treated with the CDK9 inhibitors in accordance with the present invention.

### BACKGROUND

Midline carcinomas are carcinomas arising in midline organs of subjects/patients, such as in the head, neck or mediastinum. One type of midline carcinomas are NUT midline carcinomas (subsequently referred to as “NMC”). NMC is a highly lethal cancer that has previously been described to occur in young adults and children; see French (2004) J Clin Oncology 22(20), 4135-4139. However, recent publications indicate that NMC occurs in children and adults of all ages; see French (2010) J Clin Pathol 63: 492-496.

NMC is a disease which is genetically defined by rearrangements in the nuclear protein in testis (NUT) gene on chromosome 15q14 most commonly in a balanced translocation with the BRD4 gene or the BRD3 gene. A corresponding rearrangement has first been disclosed in a cell line termed Ty-82 which had been derived from a 22-year old woman with undifferentiated thymic carcinoma; see Kuzume (1992) Int J Cancer 50, 259-264. Later, it has been found that this translocation involving rearrangement in the NUT gene is characteristic for a particularly aggressive form of a midline carcinoma and the term NUT midline carcinoma has been coined; see French (2001) Am J Pathol 159(6), 1987-1992.

NMC as a genetically defined disease does not arise from a specific organ. Most cases occur in the mediastinum and upper aerodigestive tract, but in some cases tumors have arisen in bone, bladder, abdominal retroperitoneum, pancrease and salivary glands; see French (2010), Cancer Genetics and Cytogenetics 203, 16-20 and Ziai (2010) Head and Neck Pathol 4, 163-168.

### SUMMARY

The present invention relates to a CDK9 inhibitor, especially a selective CDK9 inhibitor, for use in treating, ameliorating and/or preventing midline carcinoma. Also corresponding methods for treating, preventing or ameliorating midline carcinoma are subject of the present invention. Preferably, NUT midline carcinoma is treated with the CDK9 inhibitors in accordance with the present invention.

### BRIEF DESCRIPTION OF THE DRAWINGS

The following drawings form part of the instant specification and are included to further demonstrate certain aspects of particular embodiments herein. The embodiments may be better understood by reference to one or more of these drawings in combination with the detailed description presented herein.

FIG. 1 illustrates a proliferation inhibition profile of a potent specific CDK9 inhibitor (Cpd B1) and a selective CDK1 inhibitor (Ro-3306).

FIG. 2 illustrates CDK9 inhibition by and selectivity of described compounds.

FIG. 3 illustrates general kinase selectivity of two typical selective CDK9 inhibitors 1073485-20-7P and Cpd B1. Employing enzymatic in vitro assays on the activity of 23 different kinases.

FIG. 4 illustrates proliferation assay results of selected CDK9 inhibitors as well as other CDK standard inhibitors on various Brd4-Nut mutated as well as wild type cell lines.

FIG. 5 illustrates the expression of BrdNut fusion proteins in various cell lines (Hela, HCC2429, Ty-82, 143100, 69100 and HCC1143).

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation. In some cases, well-known methods or components have not been included in the description.

In about two thirds of NMC cases NUT is fused to BRD4 on chromosome 19; see French (2003) Cancer Res 63, 304-307 and French (2008) Oncogene 27, 2237-2242. French (2008) found that in certain cases NUT may also be fused to BRD3. Further, the authors of this document investigated the functional role of BRD-NUT fusion proteins using an siRNA assay for silencing expression. It was found that the suppression of expression of such fusion genes results in squamous differentiation and cell cycle arrest and it was concluded that BRD-NUT fusion proteins contribute to carcinogenesis. It has been suggested in the art that NUT rearrangement is a very early, possible tumour-initiating event; see French (2010) J Clin Pathol (loc. cit.).

NUT rearrangements are restricted to NMC and, therefore, the diagnosis of NMC is not in question once NUT rearrangement has been detected by immunuohistochemical testing (e.g. FISH) or by molecular testing like detection of the expression of NUT fusion genes, in particular BRD4-NUT fusion genes, BRD3-NUT fusion genes or fusions of NUT with other uncharacterized genes (termed NUT-variant fusion genes). The expression of such fusion genes goes along with corresponding NUT rearrangements. Also NMC diagnosis via detection of NUT expression with a NUT specific monoclonal antibody has been disclosed in the art; see Haack (2009) Am J Surg Pathol 33(7), 984-991. Thus, the challenge is not the diagnosis of NMC but rather the decision to perform the diagnosis on subject suspected of suffering from NMC.

Generally, it is believed that midline carcinoma, especially NMC, is a rare type of cancer; however, most cases of NMC currently go unrecognized due to its lack of characteristic histological features; see French (2010) J Clin Pathol loc. cit. NMCs are often mistaken for other cancer types such as thymic carcinoma, squamous cell carcinoma of the head and neck, lung carcinoma, Ewing sarcoma, and acute leukemia; see Schwartz (2011) Cancer Res 71(7), 2686-2696. French (2010) J Clin Pathol loc. cit. has proposed to consider any poorly differentiated, monomorphic, midline neoplasm that does not stain for lineage-specific markers for NUT rearrangement testing. Many patients with presently undiagnosed NMC would profit enormously from diagnosis and subsequent effective treatment of NMC.

Unfortunately, an effective therapy of midline carcinoma, such as NMC, is presently not available resulting in a low survival rate (1 survival out of 22 reported cases) and a mean survival of less than 1 year (9.5 months) despite aggressive chemotherapy and radiation treatment, as summarized in Table 1 of French (2010) J Clin Pathol, loc. cit. and French (2010), Cancer Genetics and Cytogenetics 203, 16-20. Further, numerous NMC tumors might not be treated at all or treatment might commence late due to a late or absent NMC diagnosis. Though reliable diagnosis of NMC is, in principle, available, there is, thus, a need in the art for the efficient treatment of midline carcinoma, especially of NMC.

Potential therapies of midline carcinoma, such as NMC, have been proposed in the art. Schwartz (loc. cit.) has investigated the mechanism underlying an NMC subtype that is characterized by the expression of the BRD4-NUT fusion gene. Schwartz found that expression of BRD4-NUT is associated with globally decreased histone deacetylation and transcriptional repression. Therefore, the authors of this document suggest the use of histone deacetylase inhibitors (HDACi) such as vorinostat and romidepsin in order to revert this effect and to thereby treat NMC. Schwartz also suggests the use of small molecule bromodomain inhibitors (Brdi) to target BRD4-NUT; yet, the authors emphasize that the most specific targeting of BRD4-NUT would be directed at NUT and that the potential difficulties in identifying deliverable NUT-directed inhibitors may be facilitated by the recent development of stapled peptides. In line with Schwartz (loc. cit.) the international patent application WO 2010/011700 describes the use of compounds, in particular histone deacetylase inhibitors that promote increased acetylation of histones for the treatment of a cancer characterized by NUT or BRD chromosomal rearrangements. Also Filippakopoulos (2010) propose the BRD4-NUT fusion as therapeutic target in NMC using a BRD4-directed inhibitor termed JQ1 (a thieno-triazolo-1,4-diazepine).

Alsarraj (2011) Cancer Res (author manuscript accepted for publication, doi:10.1158/0008-5472. CAN-10-4417) explores the role of the bromodomain-containing chromatin modifying factor BRD4 in development of cancer. Alsarraj describes that ectopic Brd4 expression represses primary tumor growth and that Brd4 activation is predictive for good outcome in human breast cancer; the authors of this document speculate that the tumor- and the metastasis-suppressive properties of Brd4 may be associated with the presence of a proline-rich region in the C-terminal part of one isoform while the extreme C-terminal domain containing a P-TEFb binding region is found to act as a metastasis enhancer. However, Alsarraj does not suggest a potential treatment of cancer and is not concerned at all with midline carcinoma, such as NMC.

Thus, the technical problem underlying the present invention is the provision of means and methods allowing the therapeutic intervention in midline carcinoma.

The technical problem is solved by provisions of the embodiments characterized in the claims.

Accordingly, the present invention relates to a CDK9 inhibitor for use in treating, ameliorating and/or preventing midline carcinoma.

In a further embodiment, the present invention relates to a method for treating, preventing or ameliorating midline carcinoma comprising the administration of a CDK9 inhibitor to a subject in need of such a treatment, prevention or amelioration. Preferably, the subject is a human.

As shown in the appended examples, it was surprisingly found that cells that comprise a rearrangement in the NUT gene are in particular susceptible to CDK9 inhibitors. The CDK9 inhibitors are also useful in the treatment of midline carcinomas in general. The examples provided herein show that CDK9 inhibitors, such as selective CDK9 inhibitors, can successfully be employed in the treatment of NUT midline carcinoma (NMC) which is, by definition, characterized by rearrangements in the NUT gene. Nothing in the art suggested the use of CDK9 inhibitors in this context. Preferably, CDK9 inhibitors Cpd B2 and Cpd B1 are used. These and further CDK9 inhibitor that may be used are described herein below in more detail. In accordance with the above, the treatment, prevention or amelioration of NUT midline carcinoma (NMC) is preferred herein. Further it is expected that the use of CDK9 inhibitors, especially of the selective CDK9 inhibitors is associated with less side effects.

The tumor cell or cancer cells of the NMC to be treated in accordance with the present invention may comprise at least one rearrangement in the NUT gene, i.e. the NMC is characterized by the presence of at least one rearrangement in the NUT gene in said tumor cell or cancer cell. The term “rearrangement in the NUT gene” refers to any rearrangement in the NUT gene that is characteristic for NUT midline carcinoma (NMC) or a rearrangement resulting in the expression of a Brd/Nut fusion protein. Exemplary “rearrangements in the NUT gene” as well as methods for their detection are known in the art (see, for example, French (2010) J Clin Pathol, loc. cit.) and also described herein. Whether a tumor or cancer cell has such a rearrangement, may be determined in an individual, isolated tumor cell or biological/medical/pathological samples, like body fluids, isolated body tissue samples and the like, wherein said samples preferably comprise cells or cell debris to be analyzed.

As mentioned above, rearrangements in the NUT gene have never been disclosed in context with susceptibility to CDK9 inhibitors, such as selective CDK9 inhibitors. At most, HDAC inhibitors, BRD inhibitors or NUT inhibitors have been proposed in context of the development of potential therapies of midline carcinomas; see Schwartz, loc. cit. In particular, Patients suffering from cancer with (a) rearrangement(s) in the NUT gene (like patients suffering from NMC) have a particularly low survival rate and a bad prognosis and known therapies are not effective. These patients and also patients suffering from midline carcinoma in general will, therefore, profit enormously from the herein provided therapy with CDK9 inhibitors.

It is also envisaged herein that one, two or more different CDK9 inhibitors (i.e. CDK9 inhibitors having different chemical formulae, optionally non-structurally related CDK9 inhibitors) may be used simultaneously. Preferred CDK9 inhibitors to be used in the present invention are described herein below.

As used herein, a kinase “inhibitor” refers to any compound capable of downregulating, decreasing, suppressing or otherwise regulating the amount and/or activity of a kinase Inhibition of these kinases can be achieved by any of a variety of mechanisms known in the art, including, but not limited to binding directly to the kinase polypeptide, denaturing or otherwise inactivating the kinase, or inhibiting the expression of the gene (e.g., transcription to mRNA, translation to a nascent polypeptide, and/or final polypeptide modifications to a mature protein), which encodes the kinase. Generally, kinase inhibitors may be proteins, polypeptides, nucleic acids, small molecules, or other chemical moieties.

As used herein the term “inhibiting” or “inhibition” refers to the ability of a compound to downregulate, decrease, reduce, suppress, inactivate, or inhibit at least partially the activity of an enzyme, or the expression of an enzyme or protein and/or the virus replication.

The term “CDK9 inhibitor” means accordingly in this context a compound capable of inhibiting the expression and/or activity of “CDK9” defined herein. A CDK9 inhibitor may, for example, interfere with transcription of a CDK9 gene, processing (e.g. splicing, export from the nucleus and the like) of the gene product (e.g. unspliced or partially spliced mRNA) and/or translation of the gene product (e.g. mature mRNA). The CDK9 inhibitor may also interfere with further modification (like phosphorylation) of the polypeptide/protein encoded by the CDK9 gene and thus completely or partially inhibit the activity of the CDK9 protein as described herein above. Furthermore, the CDK9 inhibitor may interfere with interactions of the CDK9 protein with other proteins.

In accordance with the above, the compounds according to the general formula (I) disclosed herein below as well as pharmaceutically acceptable salts thereof are used as an inhibitor for a protein kinase, preferably as an inhibitor for a cellular protein kinase.

In a preferred embodiment of this aspect said cellular protein kinase consists of Cyclin-dependent protein kinases (CDKs). The cyclin-dependent protein kinase can be selected from the group comprising: CDK1, CDK2, CDK3, CDK4, CDK5, CDK6, CDK7, CDK8, CDK9, CDK10, CDK11, CrkRS (Crk7, CDC2-related protein kinase 7), CDKL1 (cyclin-dependent kinase-like 1); KKIALRE, CDKL2 (cyclin-dependent kinase-like 2), KKIAMRE, CDKL3 (cyclin-dependent kinase-like 3), NKIAMRE, CDKL4, similar to cyclin-dependent kinase-like 1, CDC2L1 (cell division cycle 2-like 1), PITSLRE B, CDC2L1 (cell division cycle 2-like 1), PITSLRE A, CDC2L5 (cell division cycle 2-like 5), PCTK1 (PCTAIRE protein kinase 1), PCTK2 (PCTAIRE protein kinase 2), PCTK3 (PCTAIRE protein kinase 3) or PFTK1 (PFTAIRE protein kinase 1).

In a particularly preferred embodiment said cyclin-dependent protein kinase is CDK9. Thus, the compounds according to the general formula (I) as well as pharmaceutically acceptable salts thereof are, in a very preferred embodiment, used as an inhibitor for CDK9, in particular as a selective CDK9 inhibitor.

Furthermore, in another particularly preferred embodiment the compounds according to the invention show a high potency (demonstrated by a low IC₅₀ value) for inhibiting CDK9 activity. In context of the present invention, the IC₅₀ value with respect to CDK9 can be determined by the methods described in the method section of PCT patent application PCT/EP2011/001445 which is incorporated herein by reference in its entirety. Preferably, it is determined according to the method described in section 3.6 of said PCT patent application PCT/EP2011/001445.

Surprisingly it turned out that the compounds according to the general formula (I) as well as pharmaceutically acceptable salts thereof selectively inhibit CDK9 in comparison to other protein kinases and in comparison to other cyclin-dependent protein kinases. Thus, the compounds according to the general formula (I) as well as pharmaceutically acceptable salts thereof are used as selective inhibitors for CDK9.

Particularly preferred compounds of the present invention according to formula (I) show a stronger CDK9 than CDK2 inhibition. In context of the present invention, the IC₅₀ value with respect to CDK2 can be determined by the methods described in the method section of PCT patent application PCT/EP2011/001445. Preferably, it is determined according to the method described in section 3.5 of PCT/EP2011/001445.

Selectivity expresses the biologic fact that at a given compound concentration enzymes (or proteins) are affected to different degrees. In the case of enzymes selective inhibition can be defined as preferred inhibition by a compound at a given concentration. Or in other words, an enzyme is selectively inhibited over another enzyme when there is a concentration which results in inhibition of the first enzyme whereas the second enzyme is not affected. To compare compound effects on different enzymes it is crucial to employ similar assay formats, such as the LANCE assay as described in more detail below.

The inhibitors to be used herein are preferably specific for CDK9, i.e. the compounds specifically inhibit CDK9. In other words, the CDK9 inhibitors are preferably selective CDK9 inhibitors.

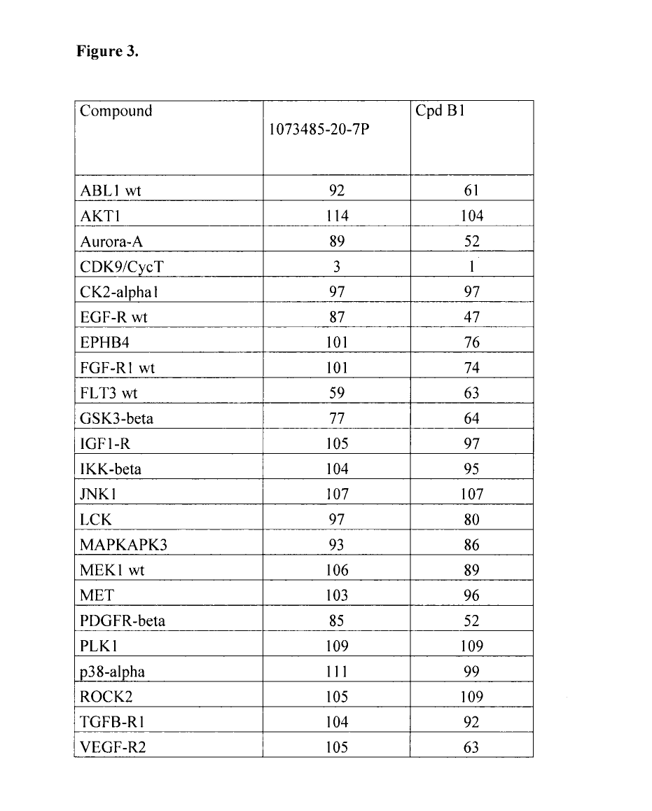

This is inter alia shown in FIG. 3 where the inhibiting effect of exemplary compounds on CDK9 is demonstrated.

A radiometric protein kinase assay (33 PanQinase® Activity Assay) was used for measuring the kinase activity of protein kinases employing exemplary CDK9 inhibitors to be used in the present invention (see FIG. 3). The low kinase activities of CDK9 show that exemplary compounds potently inhibit CDK9. Activities of other kinases are not inhibited.

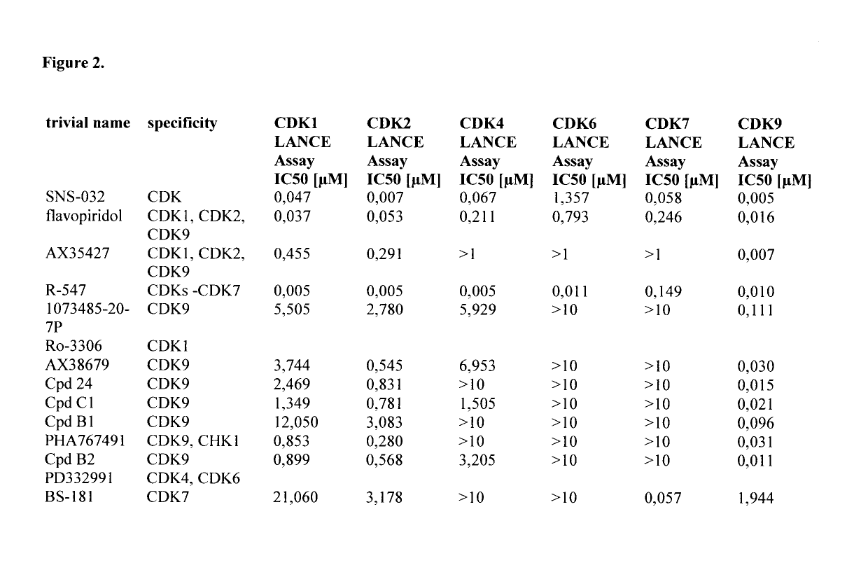

In the experimental part selectivity of the herein provided inhibitors for CDK9 is shown using, inter alia, the well known Lance Assay; see FIG. 2. The Lance assay has been described for example in Moshinsky et al.; 2003 (A widely applicable, high-throughput TR-FRET assay for the measurement of kinase autophosphorylation: VEGFR-2 as a prototype. Moshinsky D J, Ruslim L, Blake R A, Tang F. J Biomol Screen. 2003 August; 8(4):447-52). The Lance Ultra KinaSelect Assay may be used to determine half maximal inhibitory concentration (IC₅₀) of inhibitor compounds and CDK/Cyclin complexes. The principle behind this enzymatic assay is based upon the phosphorylation of the Ulight-Peptide Substrate. It is detected by using a specific EU-labeled anti-phospho peptide antibody. The binding of the Eu labeled anti-phospho peptide antibody to the phosphorylated ULight labeled peptide gives rise to a FRET-signal. Binding of an inhibitor to the kinase prevents phosphorylation of the Ulight-MBP Substrate, resulting in a loss of FRET. Based on these results, the IC50 value can be determined.

The Lance assay and the ³³ PanQinase® assay may be performed as follows:

Typically such experiments are started by generation of compounds which are serially diluted in multi titer plates in dimethylsulfoxide (DMSO). In the next step, working solutions for the enzymes, the substrates (protein and ATP separately) are generated in enzyme buffer. The preparation of the assay plate (definition of positive and negative control, reference inhibitors, test compounds and the pipetting of all solutions and compounds except the ATP working solution) is done within the next step. Finally the reaction is started by the addition of the ATP working solution. All pipetting steps can be done manually or by the help of robotics. Within the incubation of 1 h at room temperature the enzyme catalyzes the generation of phosphorylated substrate. This reaction is more ore less inhibited by the added compounds. Finally, to stop the reaction and to detect phosphorylated substrate detection buffer (see material and methods) is added followed by another incubation of 1 h. The data is evaluated by measuring the FRET-Signal. Data is processed by subtraction of the background signal (negative control) from all investigated activities. These activities are set into relation to the positive control. Altogether this is shown by the following equation:

resulting activity (%)=100×[(signal of compound−signal of negative control)/(signal of positive control−signal of negative control)]

Further analysis steps include the determination of IC50 values by using the activities of a dose response experiment and an algorithm (equation #205 in Excel fit) for calculation.

A similar experimental procedure is performed when the resulting activity within ³³ PanQinase® assay is done. In advance buffers are prepared but in this case the pipetting sequence is first ATP solution diluted with assay buffer, DMSO or compound solution. The reaction (1 h at 30° C.) is started by addition of a substrate-kinase mix. During the incubation the kinase phosphorylates the substrate (different for each kinase). Due to the fact that the ATP solution contains ³³P-labelled ATP the substrate proteins are labeled with ³³P. The reaction is stopped by addition of excess H₃PO₄. If the reaction is performed in plates binding substrate proteins, said plates are washed to reduce unspecific signals (mainly not used ATP). The incorporation of ³³P into substrate proteins is a direct measure of activity of the respective kinase. Therefore, the incorporated radioactivity is detected by scintillation counting. Data is evaluated, processed and analyzed as described for the LANCE assays.

From FIG. 2 it can be deduced that a known CDK7-inhibitor (BS-181) has an IC50 value of 1.944 in the CDK9 Lance Assay. As shown in the experimental part, the IC50 value determined for exemplary selective CDK9 inhibitors, for example according to the Lance Assay, is low, preferably below 0.2 μM, more preferably, below 0.15 μM, 0.14 μM, 0.13 μM, 0.12 μM or even lower. More preferably, the IC50 value is below 0.1 μM, 0.095 μM, 0.090 μM, 0.085 μM, 0.080 μM, 0.075 μM, 0.070 μM, 0.065 μM, 0.060 μM, 0.055 μM, 0.050 μM, 0.045 μM, 0.040 μM, 0.035 μM, 0.030 μM, or even below 0.025 μM, wherein the lower values are preferred over the higher values. Even more preferably, the IC50 value is below 0.024 μM, 0.023 μM, 0.022 μM, 0.021 μM, 0.020 μM, 0.019 μM, 0.018 μM, 0.017 μM, 0.016 μM, 0.015 μM, 0.014 μM, 0.013 μM, 0.012 μM, or 0.011 μM. The IC50 value may even be lower, for example, below 0.010 μM, 0.009 μM, 0.008 μM, 0.007 μM, 0.006 μM, or 0.005 μM. Generally, the lower values are preferred herein over the higher values.

It is preferred herein that the ratio of IC50 values of selective CDK9-inhibitors determined according to the CDK9 Lance assay and IC50 values of selective CDK9-inhibitors determined according to the CDK1 Lance assay, CDK2 Lance assay, CDK4 Lance assay, and/or the CDK6 Lance assay is about 1:10 or lower. A ratio of 1:10 or lower also indicates selectivity of the inhibitor for CDK9. More preferred is a ratio of 1:10, 1:20, 1:30, 1:40, 1:50, 1:60, 1:70, 1:80, 1:90 or 1:100 or even lower.

The following selective CDK9 inhibitors are preferably used in accordance with the present invention; these and further selective CDK9 inhibitors for use in the present invention are described in PCT/EP2011/001445, EP10075131.2 (filing date 22 Mar. 2010) EP11075037.9 (filing date 2 Mar. 2011) and EP11075038.7 (filing date 2 Mar. 2011) which are incorporated herein by reference in their entirety.

The disubstituted triazine compounds to be used according to the present invention are defined by the general formula (I)

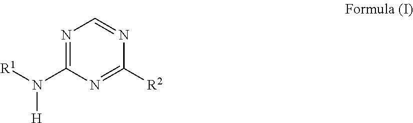

wherein

### R¹ is

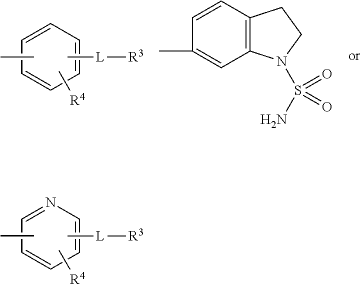

L is a bond or —CR⁵R⁶—, —CR⁵R⁶—CR⁷R⁸—, —CR⁵R⁶—CR⁷R⁸—CR⁹R¹⁰—, —CR⁵R⁶—CR⁷R⁸—CR⁹R¹⁰—CR¹¹R¹²—;
R⁵-R¹² represent independently of each other —H, —CH₃, —C₂H₅, —C₃H₇, —F, —Cl, —Br, —I;
R³ is selected from —H, —NO₂, —NH₂, —CN, —F, —Cl, —Br, —I, —CH₃, —C₂H₅, -Ph, —C₃H₇, —CH(CH₃)₂, —C₄H₉, —CH₂—CH(CH₃)₂, —CH(CH₃)—C₂H₅, —C(CH₃)₃, —O—CH₃, —O—C₂H₅, —O—C₃H₇, —O—CH(CH₃)₂, —O—C₄H₉, —O—CH₂CH₃)₂, —O—CH(CH₃)—C₂H₅, —O—C(CH₃)₃, —CR¹³R¹⁴R²¹, —CR¹³R¹⁴—CR¹⁵R¹⁶R²¹, —O—CR¹³R¹⁴R²¹, CR¹³R¹⁴—CR¹⁵R¹⁶—CR¹⁷R¹⁸R²¹, —CR¹³R¹⁴—CR¹⁵R¹⁶—CR¹⁷R¹⁸—CR¹⁹R²⁰R²¹, —O—CR¹³R¹⁴—CR¹⁵R¹⁶R²¹, —O—CR¹³R¹⁴—CR¹⁵R¹⁶—CR¹⁷R¹⁸R²¹, —SO₂R²², —CONR²³R²⁴, —NR²⁵COR²², —O—CR¹³R¹⁴—CR¹⁵R¹⁶—CR¹⁷R¹⁸—CR¹⁹R²⁰R²¹, NR²⁵SO₂NR²³R²⁴, —NR²⁵SO₂R²², —NR²⁵CONR²³R²⁴, —SO₂NR²³R²⁴, —SO(NR²⁶)R²⁷, —NH—CO—NH-Ph;
R¹³-R²¹, R²⁹-R³² and R³³-R⁴⁸ represent independently of each other —H, —F, —Cl, —Br, —I;
R²⁶ is —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CR¹³R¹⁴R²¹, —COR²⁸, —CR¹³R¹⁴—CR¹⁵R¹⁶R²¹, —CR¹³R¹⁴—CR¹⁵R¹⁶—CR¹⁷R¹⁸—CR¹⁹R²⁰—CR²⁹R³⁰R²¹, —CR¹³R¹⁴—CR¹⁵R¹⁶—CR¹⁷R¹⁸R²¹, —CR¹³R¹⁴—CR¹⁵R¹⁶—CR¹⁷R¹⁸—CR¹⁹R²⁰R²¹, —CR¹³R¹⁴—CR¹⁵R¹⁶—CR¹⁷R¹⁸—CR¹⁹R²⁰—CR²⁹R³⁰—CR³¹R³²R²¹, —COOR²⁸,

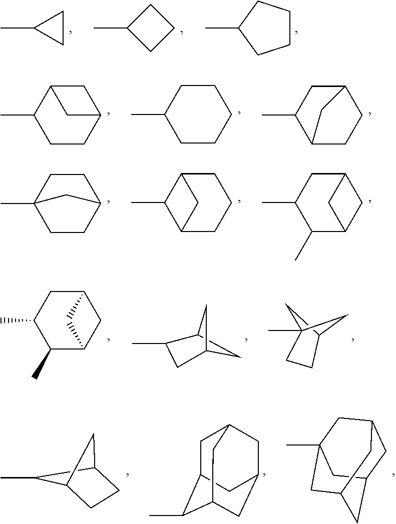

these C₃-C₆-cycloalkyl groups may further be substituted by one, two, three, four, five or more substituents selected from the group consisting of R³³-R⁴⁸;
R²², R²⁷, and R²⁸ are independently selected from —CR⁴⁹R⁵⁰R⁵¹, —CR⁴⁹R⁵⁰—CR⁵²R⁵³R⁵¹, —CR⁴⁹R⁵⁰—CR⁵²R⁵³—CR⁵⁴R⁵⁵—CR⁵⁶R⁵⁷—CR⁵⁸R⁵⁹R⁵¹, —CR⁴⁹R⁵⁰—CR⁵²R⁵³—CR⁵⁴R⁵⁵R⁵¹, CR⁴⁹R⁵⁰—CR⁵²R⁵³, —CR⁵⁴R⁵⁵—CR⁵⁶R⁵⁷R⁵¹, —CR⁴⁹R⁵⁰—CR⁵²R⁵³—CR⁵⁴R⁵⁵—CR⁵⁶R⁵⁷—CR⁵⁸R⁵⁹—CR⁶⁰R⁶¹R⁵¹, —CH₂Ph; —CH₂Ph the phenyl group of which may further be substituted by one, two, three, four or five substituents selected from the group consisting of R⁵-R¹²;
C₃-C₆-cycloalkyl groups listed for R²⁶, which may further be substituted by one, two, three, four, five or more substituents selected from the group consisting of R³³-R⁴⁸;
R⁴⁹-R⁶¹ represent independently of each other —H, —CH₃, —C₂H₅, —C₃H₇, —C₄H₉, —F, —Cl, —Br, —I, —OH, —NO₂, —NH₂;
R²³ and R²⁴ are independently selected from —H, —CR⁴⁹R⁵⁰R⁵¹, —CR⁴⁹R⁵⁰—CR⁵²R⁵³R⁵¹, —CR⁴⁹R⁵⁰—CR⁵²R⁵³—CR⁵⁴R⁵⁵—CR⁵⁶R⁵⁷—CR⁵⁸R⁵⁹R⁵¹, —CR⁴⁹R⁵⁰—CR⁵²R⁵³—CR⁵⁴R⁵⁵R⁵¹, —CR⁴⁹R⁵⁰—CR⁵²R⁵³—CR⁵⁴R⁵⁵—CR⁵⁶R⁵⁷R⁵¹, —CR⁴⁹R⁵⁰—CR⁵²R⁵³—CR⁵⁴R⁵⁵—CR⁵⁶R⁵⁷—CR⁵⁸R⁵⁹—CR⁶⁰R⁶¹R⁵¹, —CR⁴⁹R⁵⁰—CR⁵²R⁵³—O—R^{51′}, —CR⁴⁹R⁵⁰—CR⁵²R⁵³—CR⁵⁴R⁵⁵—O—R^{51′}, —CR⁴⁹R⁵⁰—CR⁵²R⁵³—NR^{51′}R^{51″}, —CR⁴⁹R⁵⁰—CR⁵²R⁵³—CR⁵⁴R⁵⁵—NR^{51′}R^{51″}, —CR⁴⁹R⁵⁰—CR⁵²R⁵³—CR⁵⁴R⁵⁵—CR⁵⁶R⁵⁷—NR^{51′}R^{51″}, —CR⁴⁹R⁵⁰—CR⁵²R⁵³—CR⁵⁴R⁵⁵—CR⁵⁶R⁵⁷—CR⁵⁸R⁵⁹—NR^{51′}R^{51″}, phenyl, substituted phenyl, benzyl, substituted benzyl, or both residues R²³ and R²⁴ together form with the nitrogen atom to which they are attached a azetidine, pyrrolidine, piperidine, piperazine, azepane, or morpholine ring;
R^{51′} and R^{51″} represent independently of each other —H, —CH₃, —C₂H₅, —C₃H₇, —C₄H₉, —CH₂Ph, —COOC(CH₃)₃, —COOCH₃, —COOCH₂CH₃, —COOCH₂CH₂CH₃, —COOCH(CH₃)₂, —COOCH₂Ph, —COCH₃;
and R²⁵ is selected from —H, —CH₃, —C₂H₅, —C₃H₇, —CH(CH₃)₂, —C₄H₉, —CH₂—CH(CH₃)₂, —CH(CH₃)—C₂H₅ or —C(CH₃)₃;
R⁴ is selected from —H, —NO₂, —NH₂, —CN, —F, —Cl, —Br, —I, —CONH₂, —SO₂CH₃, —SO₂C₂H₅, —SO₂C₃H₇, —NH—SO₂—CH₃, —NH—SO₂—C₂H₅, —NH—SO₂—C₃H₇, —NHCO—CH₃, —NHCO—C₂H₅, —NHCO—C₃H₇, —SO₂NR²³R²⁴, —CH₂—SO₂NR²³R²⁴, —C₂H₄—SO₂NR²³R²⁴, —C₃H₆—SO₂NR²³R²⁴, —SO₂NH₂, —CH₂—SO₂NH₂, —C₂H₄—SO₂NH₂, —C₃H₆—SO₂NH₂,

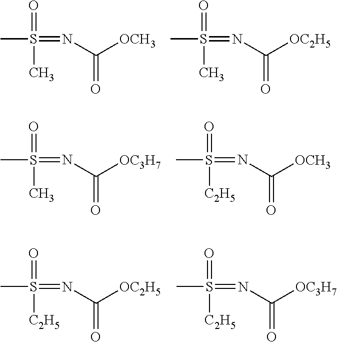

—CR⁶²R⁶³R⁶⁴, —CR⁶²R⁶³—CR⁶⁵R⁶⁶—CR⁶⁷R⁶⁸—CR⁶⁹R⁷⁰R⁶⁴, —O—CR⁶²R⁶³—CR⁶⁵R⁶⁶R⁶⁴, —O—CR⁶²R⁶³—CR⁶⁵R⁶⁶—CR⁶⁷R⁶⁸R⁶⁴, —CR⁶²R⁶³—CR⁶⁵R⁶⁶—CR⁶⁷R⁶⁸R⁶⁴, —O—CR⁶²R⁶³—CR⁶⁵R⁶⁶—CR⁶⁷R⁶⁸—CR⁶⁹R⁷⁰R⁶⁴, —CR⁶²R⁶³—CR⁶⁵R⁶⁶R⁶⁴, —O—CR⁶²R⁶³—CR⁶⁵R⁶⁶—CR⁶⁷R⁶⁸—CR⁶⁹R⁷⁰—CR⁷¹R⁷²R⁶⁴, —O—CR⁶²R⁶³R⁶⁴, —O—CR⁶²R⁶³—CR⁶⁵R⁶⁶—CR⁶⁷R⁶⁸—CR⁶⁹R⁷⁰—CR⁷¹R⁷²—CR⁷³R⁷⁴R⁶⁴, —CR⁶²R⁶³—CR⁶⁵R⁶⁶—CR⁶⁷R⁶⁸—CR⁶⁹R⁷⁰—CR⁷¹R⁷²R⁶⁴, —CR⁶²R⁶³—CR⁶⁵R⁶⁶—CR⁶⁷R⁶⁸—CR⁶⁹R⁷⁰—CR⁷¹R⁷²—CR⁷³R⁷⁴R⁶⁴, —OCH₂Ph,

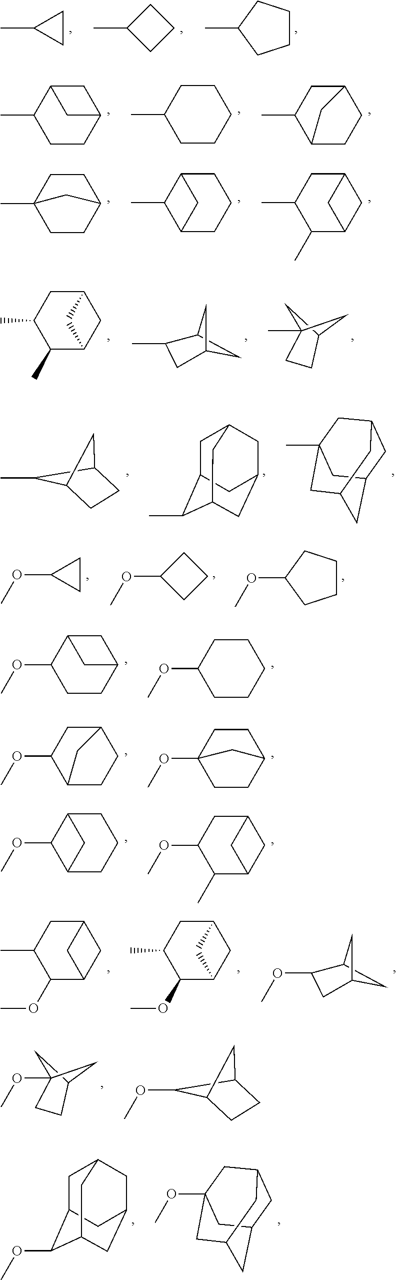

these C₃-C₆-cycloalkoxy groups and C₃-C₆-cycloalkyl groups may further be substituted by one, two, three, four, five or more substituents selected from the group consisting of R³³-R⁴⁸;
R⁶²-R⁷⁴ represent independently of each other —H, cyclo-C₃H₅, cyclo-C₄H₇, -cyclo-C₅H₉, —CR⁷⁵R⁷⁶R⁷⁷, —CR⁷⁵R⁷⁶—CR⁷⁸R⁷⁹R⁷⁷, —CR⁷⁵R⁷⁶—CR⁷⁸R⁷⁹—CR⁸⁰R⁸¹R⁷⁷, —CR⁷⁵R⁷⁶—CR⁷⁸R⁷⁹—CR⁸⁰R⁷⁹—CR⁸²R⁸¹R⁷⁷, —F, —Cl, —Br, —I, -Ph;
R⁷⁵-R⁸² represent independently of each other —H, —F, —Cl, —Br, —I, —NH₂;
R⁴ together with R²², R²³, R²⁴, or R²⁵ may form a group —CH₂CH₂— or —CH₂CH₂CH₂— if R⁴ is attached ortho to -L-R³;

### R² is

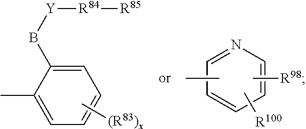

R⁸³ is selected from —H, —OH, —NO₂, —CN, —F, —Cl, —Br, —I, —NR^{23′}R^{24′}, —CF₃, —CR⁶²R⁶³R⁶⁴, —CR⁶²R⁶³—NR^{23′}R^{24′}, —CR⁶²R⁶³—CR⁶⁵R⁶⁶R⁶⁴, —CR⁶²R⁶³—CR⁶⁵R⁶⁶—NR^{23′}R^{24′}, —CR⁶²R⁶³—CR⁶⁵R⁶⁶—CR⁶⁷R⁶⁸R⁶⁴, —CR⁶²R⁶³—CR⁶⁵R⁶⁶—CR⁶⁷R⁶⁸—NR^{23′}R^{24′}, —O—CR⁶²R⁶³R⁶⁴, —O—CR⁶²R⁶³—CR⁶⁵R⁶⁶R⁶⁴, —O—CR⁶²R⁶³—CR⁶⁵R⁶⁶—CR⁶⁷R⁶⁸R⁶⁴, —CHO, —CH₂OH, —CR^{23′}O, CH₂OR^{23′};
R^{23′} and R^{24′} represent independently of each other —H, —CH₃, —C₂H₅, —C₃H₇, —CH(CH₃)₂, —C₄H₉, —CH₂—CH(CH₃)₂, —CH(CH₃)—C₂H₅, —C(CH₃)₃; -(cyclo-C₃H₅);
x is a value between 0 and 3;
B is a bond, —CR⁸⁶R⁸⁷—, —CR⁸⁶R⁸⁷—CR⁸⁸R⁸⁹—, —CR⁸⁶R⁸⁷—CR⁸⁸R⁸⁹—CR⁹⁰R⁹¹—, —CR⁸⁶R⁸⁷—CR⁸⁸R⁸⁹—CR⁹⁰R⁹¹—CR⁹²R⁹³—, —CR⁸⁶R⁸⁷—CR⁸⁸R⁸⁹—CR⁹⁰R⁹¹—CR⁹²R⁹³—CR⁹⁴R⁹⁵—, —CR⁸⁶R⁸⁷—CR⁸⁸R⁸⁹—CR⁹⁰R⁹¹—CR⁹²R⁹³—CR⁹⁴R⁹⁵—CR⁹⁶R⁹⁷—;
R⁸⁶-R⁹⁷ represent independently of each other —H, —CH₃, —C₂H₅, —C₃H₇, —C₄H₉, —F, —Cl, —Br, —I;
Y is a bond, —O—, —S—, —SO—, —SO₂—, —SO₂NH—, —NHSO₂—, —CO—, —COO—, —OOC—, —CONH—, —NHCO—, —NH—, —N(CH₃)—, —NH—CO—NH—, —O—CO—NH—, —NH—CO—O—;
R⁸⁴ is selected from a bond, —CR⁸⁶R⁸⁷—, —CR⁸⁶R⁸⁷—CR⁸⁸R⁸⁹—CR⁹⁰R⁹¹—, —CR⁸⁶R⁸⁷—CR⁸⁸R⁸⁹—CR⁹⁰R⁹¹—CR⁹²R⁹³—, —CR⁸⁶R⁸⁷—CR⁸⁸R⁸⁹—CR⁹⁰R⁹¹—CR⁹²R⁹³—CR⁹⁴R⁹⁵—, —CR⁸⁶R⁸⁷—CR⁸⁸R⁸⁹—, —CR⁸⁶R⁸⁷—CR⁸⁸R⁸⁹—CR⁹⁰R⁹¹—CR⁹²R⁹³—CR⁹⁴R⁹⁵—CR⁹⁶R⁹⁷—;
R⁸⁵ is selected from
(i) —H, —OH, —OCH₃, —OC₂H₅, —OC₃H₇, —O-cyclo-C₃H₅, —OCH(CH₃)₂, —OC(CH₃)₃, —OC₄H₉, -Ph, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —SC₄H₉, —NO₂, —F, —Cl, —Br, —I, —P(O)(OH)₂, —P(O)(OCH₃)₂, —P(O)(OC₂H₅)₂, —P(O)(OCH(CH₃)₂)₂, —Si(CH₃)₂(C(CH₃)₃), —Si(C₂H₅)₃, —Si(CH₃)₃, —CN, —CHO, —COCH₃, —COC₂H₅, —COC₃H₇, —CO-cyclo-C₃H₅, —COCH(CH₃)₂, —COC(CH₃)₃, —COC₄H₉, —COOH, —COOCH₃, —COOC₂H₅, —COOC₃H₇, —COOC₄H₉, —COO-cyclo-C₃H₅, —COOCH(CH₃)₂, —COOC(CH₃)₃, —OOC—CH₃, —OOC—C₂H₅, —OOC—C₃H₇, —OOC—C₄H₉, —OOC-cyclo-C₃H₅, —OOC—CH(CH₃)₂, —OOC—C(CH₃)₃, —CONR^{23′}R^{24′}, —NHCOCH₃, —NHCOC₂H₅, —NHCOC₃H₇, —NHCO-cyclo-C₃H₅, —NHCO—CH(CH₃)₂, —NHCOC₄H₉, —NHCO—C(CH₃)₃, —NHCO—OCH₃, —NHCO—OC₂H₅, —NHCO—OC₃H₇, —NHCO—O-cyclo-C₃H₅, —NHCO—OC₄H₉, —NHCO—OCH(CH₃)₂, —NHCO—OC(CH₃)₃, —NHCO—OCH₂Ph, —NR²³R²⁴, —CF₃, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C₄H₉, —SO₂C(CH₃)₃, —SO₃H, —SO₂NR^{23′}R^{24′}, —OCF₃, —OC₂F₅, —O—COOCH₃, —O—COOC₂H₅, —O—COOC₃H₇, —O—COO-cyclo-C₃H₅, —O—COOC₄H₉, —O—COOCH(CH₃)₂, —O—COOCH₂Ph, —O—COOC(CH₃)₃, —NH—CO—NH₂, —NH—CO—NHCH₃, —NH—CO—NHC₂H₅, —NH—CO—NHC₃H₇, —NH—CO—NHC₄H₉, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(C₄H₉)₂, —NH—CO—N(cyclo-C₃H₅)₂, —NH—CO—N[CH(CH₃)₂]₂, —NH—CO—N[C(CH₃)₃]₂, —NH—C(═NH)—NH₂, —NH—C(═NH)—NHCH₃, —NH—C(═NH)—NHC₂H₅, —NH—C(═NH)—NHC₃H₇, —NH—C(═NH)—NHC₄H₉, —NH—C(═NH)—NH-cyclo-C₃H₅, —OCH₂-cyclo-C₃H₅, —NH—C(═NH)—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —NH—C(═NH)—N(C₄H₉)₂, —NH—C(═NH)—N[CH(CH₃)₂]₂, —NH—C(═NH)—N[C(CH₃)₃]₂, —O—CO—NH₂, —O—CO—NHCH₃, —O—CO—NHC₂H₅, —O—CO—NHC₃H₇, —O—CO—NHC₄H₉, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(C₄H₉)₂, —O—CO—N(cyclo-C₃H₅)₂, —O—CO—N[CH(CH₃)₂]₂, —O—CO—N[C(CH₃)₃]₂,
(ii) an aromatic or heteroaromatic mono- or bicyclic ring selected from 2-thienyl, 3-thienyl, 2-furanyl, 3-furanyl, 2-oxazolyl, 3-oxazolyl, 4-oxazolyl, 2-thiazolyl, 3-thiazolyl, 4-thiazolyl, 1-pyrazolyl, 3-pyrazolyl, 4-pyrazolyl, 5-pyrazolyl, 1-imidazolyl, 2-imidazolyl, 4-imidazolyl, 5-imidazolyl, phenyl, 1-naphthyl, 2-naphthyl, 2-pyridyl, 3-pyridyl, 4-pyridyl, 2-pyrimidinyl, 4-pyrimidinyl, 5-pyrimidinyl, 2-pyrazinyl, 3-pyridazinyl, 4-pyridazinyl, 1,3,5-triazin-2-yl,

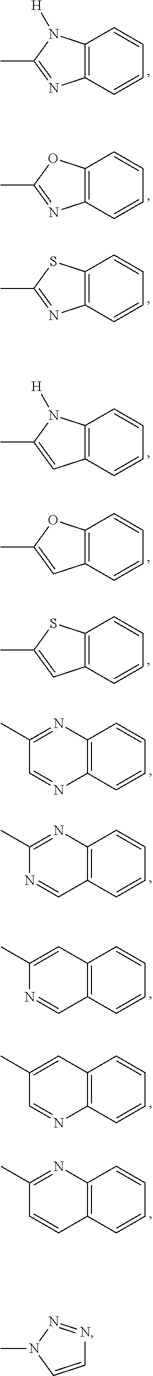

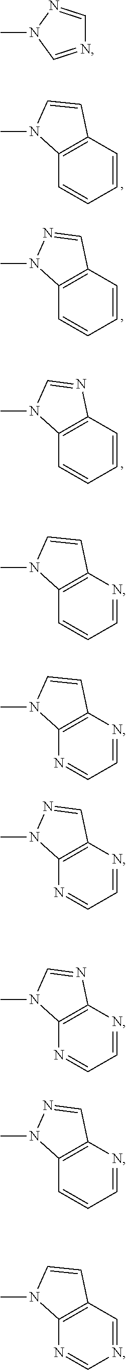

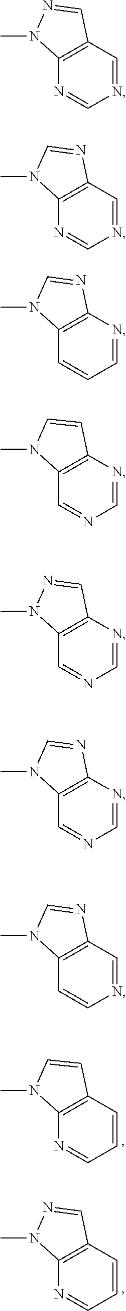

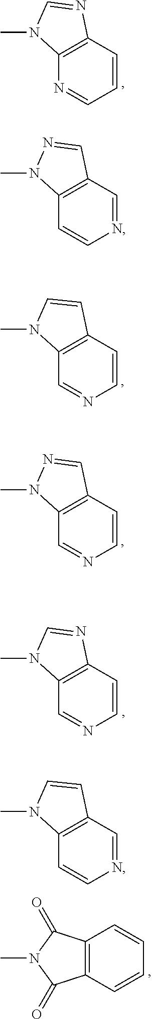

which optionally may be substituted by one or two substituents selected from —F, —Cl, —Br, —I, —OCH₃, —CH₃, —NO₂, —CN, —CF₃;
(iii) a saturated ring selected from

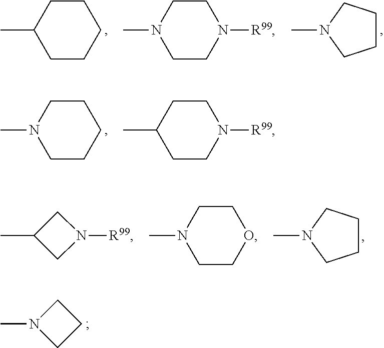

R⁹⁹ represents —H, —CH₃, —CH₂Ph, —COOC(CH₃)₃, —COOCH₃, —COOCH₂CH₃, —COOCH₂CH₂CH₃, —COOCH(CH₃)₂, —COOCH₂Ph, —COCH₃;
the group —B—Y—R⁸⁴—R⁸⁵ together with one substituent R⁸³ may form a group —OCH₂O—, if R⁸³ is attached in position ortho to —B—Y—R⁸⁴—R⁸⁵;
with the proviso that R⁸³ is not —H, if the group —B—Y—R⁸⁴—R⁸⁵ is hydrogen.
R⁹⁸ is selected from —NO₂, —CN, —F, —Cl, —Br, —I, —NH₂, —OH, —CR⁶²R⁶³—CR⁶⁵R⁶⁶—CR⁶⁷R⁶⁸—CR⁶⁹R⁷⁰R⁶⁴, —O—CR⁶²R⁶³R⁶⁴, —O—CR⁶²R⁶³—CR⁶⁵R⁶⁶R⁶⁴, —O—CR⁶²R⁶³—CR⁶⁵R⁶⁶—CR⁶⁷R⁶⁸R⁶⁴, —O—CR⁶²R⁶³—CR⁶⁵R⁶⁶—CR⁶⁷R⁶⁸—CR⁶⁹R⁷⁰R⁶⁴, —O—CR⁶²R⁶³—CR⁶⁵R⁶⁶—CR⁶⁷R⁶⁸—CR⁶⁹R⁷⁰—CR⁷¹R⁷²R⁶⁴, —CR⁶²R⁶³—CR⁶⁵R⁶⁶—CR⁶⁷R⁶⁸R⁶⁴, —O—CR⁶²R⁶³—CR⁶⁵R⁶⁶—CR⁶⁷R⁶⁸—CR⁶⁹R⁷⁰—CR⁷¹R⁷²—CR⁷³R⁷⁴R⁶⁴, —CR⁶²R⁶³—CR⁶⁵R⁶⁶R⁶⁴, —CR⁶²R⁶³—O—CR⁶⁵R⁶⁶—CR⁶⁷R⁶⁸—CR⁶⁹R⁷⁰R⁶⁴, —CR⁶²R⁶³—O—CR⁶⁵R⁶⁶—CR⁶⁷R⁶⁸R⁶⁴, CR⁶²R⁶³—O—CR⁶⁵R⁶⁶—CR⁶⁷R⁶⁸—CR⁶⁹R⁷⁰—CR⁷¹R⁷²R⁶⁴, —CR⁶²R⁶³—O—CR⁶⁵R⁶⁶R⁶⁴, CR⁶²R⁶³—O—CR⁶⁵R⁶⁶—CR⁶⁷R⁶⁸—CR⁶⁹R⁷⁰R⁷¹R⁷²—CR⁷³R⁷⁴R⁶⁴, CR⁶²R⁶³R⁶⁴, CR⁶²R⁶³—CR⁶⁵R⁶⁶—CR⁶⁷R⁶⁸—CR⁶⁹R⁷⁰—CR⁷¹R⁷²R⁶⁴, —OCH₂Ph, —OCH₂—CH₂-Ph, —CH₂—O—CH₂-Ph, —CR⁶²R⁶³—CR⁶⁵R⁶⁶—CR⁶⁷R⁶⁸—CR⁶⁹R⁷⁰—CR⁷¹R⁷²—CR⁷³R⁷⁴R⁶⁴;
with the proviso that R⁹⁸ is attached to a position ortho to the bond between the pyridine and the triazine ring if R⁹⁸ is not an amino group in para position to the bond between the pyridine and the triazine ring;
R¹⁰⁰ is selected from —H, —NO₂, —CN, —F, —Cl, —Br, —I, —NH₂, —OH, —CF₃, —CH₃, —CH₂CH₃, —CH₂CH₂CH₃, —CH(CH₃)₂, —OCH₃, —OCH₂CH₃, —OCH₂CH₂CH₃, —OCH(CH₃)₂, —OCF₃, —OCH₂Ph;
and with the proviso that if R¹ is a phenyl moiety and R² is also a phenyl moiety a chloro substituent is only allowed on the R¹ phenyl moiety or on the R² phenyl moiety but not on both simultaneously;
and with the proviso that the compound 4-[4-(2-benzoylaminophenyl)-[1,3,5]triazin-2-ylamino]benzamide is excluded;
and enantiomers, stereoisomeric forms, mixtures of enantiomers, diastereomers, mixtures of diastereomers, prodrugs, hydrates, solvates, acid salt forms, tautomers, and racemates of the above mentioned compounds and pharmaceutically acceptable salts or salts of solvates thereof.

The expression prodrug is defined as a substance, which is applied in an inactive or significantly less active form. Once applied and incorporated, the prodrug is metabolized in the body in vivo into the active compound.

The expression tautomer is defined as an organic compound that is interconvertible by a chemical reaction called tautomerization. Tautomerization can be catalyzed preferably by bases or acids or other suitable compounds.

Preferred are compounds having the general formula (I):

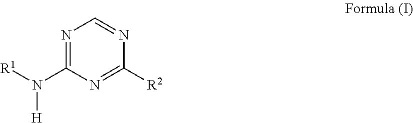

wherein

R¹ represents

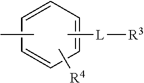

in which

L is a bond, —CH₂—, —CH₂CH₂—, or —CF₂—, particularly preferred —CH₂—;

R³ is —SO₂NH₂, —SO₂NH(CH₃), —SO₂N(CH₃)₂, —SO₂NH(CH₂CH₂OCH₃), —NHSO₂CH₃, —NHSO₂CH₂CH₃, —NHSO₂CH₂CH₂CH₃, —NHSO₂CF₃, —SO₂CH₃, —NHSO₂NH₂, —SO(NH)CH₃, particularly preferred —SO₂NH₂;

R⁴ is —H, —CH₃, —F, —Cl, or —CF₃, particularly preferred —H;

R² represents

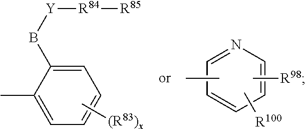

in which the group —B—Y—R⁸⁴—R⁸⁵ is —OCH₃, —OCH₂CH₃, —OCH₂CH₂CH₃, —OCH₂CH₂CH₂CH₃, —OCH(CH₃)₂, —OPh, —OCH₂Ph, —OCH₂(4-pyridyl), particularly preferred —OCH₃;

R⁸³ is —H, —F, or —Cl;

x is 0, 1, or 2;

R⁹⁸ is —OCH₃ and R¹⁰⁰ is —H, provided that R⁹⁸ is attached to a position ortho to the bond between the pyridine and the triazine ring.

In more preferred compounds of Formula (I)

the substituent -L-R³ is —SO₂NH₂, —CH₂SO₂NH₂, —CH₂CH₂SO₂NH₂, —CF₂SO₂NH₂, —NHSO₂NH₂, —CH₂NHSO₂NH₂, —SO₂CH₃, —SO(NH)CH₃, —CH₂SO(NH)CH₃,

and R⁴ is —H;

R² is 2-methoxyphenyl, 4-fluoro-2-methoxyphenyl, or 6-fluoro-2-methoxyphenyl.

Preferred are compounds of general formula (I), wherein R¹ is

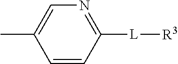

and wherein L is a bond or is —CH₂— or —CH₂CH₂— and R³ has the meanings as defined herein and more preferably R³ represents —SO₂R²² or —SO₂NR²³R²⁴, wherein R²², R²³ and R²⁴ have the meanings as defined herein and preferably R²², R²³ and R²⁴ represent independently of each other

—H, —CF₃, —CH₃, —CH₂CH₃, —CH₂CH₂CH₃, —CH₂CH₂CH₂CH₃, —CH(CH₃)₂, —CH₂—NH₂, —CH₂—CH₂—NH₂, —CH₂—CH₂—CH₂—NH₂, —CH₂—CH₂—CH₂—CH₂—NH₂, —CH₂NH—CO—O—C(CH₃)₃, —CH₂—CH₂NH—CO—O—C(CH₃)₃, —CH₂—CH₂—CH₂NH—CO—O—C(CH₃)₃, —CH₂—CH₂—CH₂—CH₂—NH—CO—O—C(CH₃)₃.

Also preferred are compounds of general formula (I), wherein L is a bond, —CH₂—, —CH₂CH₂—, —CH₂CH₂CH₂—, or —CF₂—, more preferred —CH₂— or —CH₂CH₂—.

Preferred are compounds of general formula (I), wherein R² is

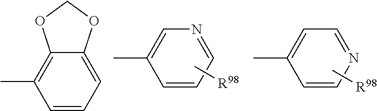

If residue R² is a phenyl ring, it is preferred that the substituent B—Y—R⁸⁴—R⁸⁵ in ortho position of the linkage to the triazine core is not hydrogen and if that substituent is hydrogen, R⁸³ is not hydrogen and moreover that at least one substituent R⁸³ is in ortho position of the linkage to the triazine core. Thus one substituent of B—Y—R⁸⁴—R⁸⁵ and R⁸³ has to be different from hydrogen so that R² cannot be an unsubstituted phenyl ring. Moreover it is preferred that R⁸⁵ is not —H, if B, Y and R⁸⁴ are bonds and R⁸³ is different from hydrogen. If two substituents are present, it is preferred that the second substituent is in meta position or para position of the linkage to the triazine core. If a third substituent is present the substitution pattern 2, 3, 5 or 2, 3, 4 are preferred. Fluorine is a preferred second and/or third substituent and is preferably in meta or para position of the linkage to the triazine core. Thus, the following residues R² are preferred:

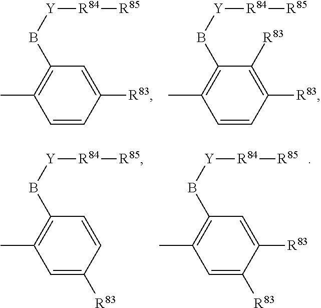

If residue R² is a pyridyl ring it is preferred that one substituent of R⁹⁸ is in ortho position of the linkage to the triazine core. Preferred are the following R² residues:

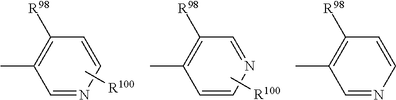

Also preferred are compounds of general formula (I), wherein R⁸⁵ is

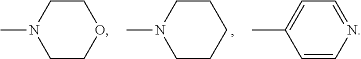

R³ is preferably selected from —H, —NO₂, —NH₂, —CN, —F, —Cl, —Br, —I, —CH₃, —C₂H₅, -Ph, —C₃H₇, —CH(CH₃)₂, —C₄H₉, —CH₂—CH(CH₃)₂, —CH(CH₃)—C₂H₅, —C(CH₃)₃, —O—CH₃, —O—C₂H₅, —O—C₃H₇, —O—CH(CH₃)₂, —O—C₄H₉, —O—CH₂—CH(CH₃)₂, —O—CH(CH₃)—C₂H₅, —O—C(CH₃)₃, —SO₂R²² and —SO₂NR²³R²⁴.

R²⁶ is preferably selected from —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, -cyclo-C₃H₅, -cyclo-C₄H₇ and -cyclo-C₅H₉.

Moreover compounds of general formula (I), wherein R²², R²³, R²⁴, R²⁷ and R²⁸ are independently of each other selected from —H, —CH₃, —C₂H₅, —C₃H₇, —C₄H₉ or —CH₂Ph.

Preferably R⁶²-R⁷⁴ represent independently of each other —H, -Ph, -cyclo-C₃H₅, -cyclo-C₄H₇, —CH₃, —C₂H₅, —C₃H₇, —C₄H₉, -cyclo-C₅H₉, —F, —Cl, —Br or —I.

Furthermore preferred are compounds of general formula (I), wherein R⁴ is selected from —H, —NO₂, —NH₂, —CN, —F, —Cl, —Br, —I, -cyclo-C₃H₅, -cyclo-C₄H₇, -cyclo-C₅H₉, —CH₃, —C₂H₅, —C₃H₇, —CH(CH₃)₂, —C₄H₉, —CONH₂, —SO₂CH₃, —SO₂C₂H₅, —SO₂C₃H₇, —NH—SO₂—CH₃, —NH—SO₂—C₂H₅, —NH—SO₂—C₃H₇, —NHCO—CH₃, —NHCO—C₂H₅, —NHCO—C₃H₇, —SO₂NR²³R²⁴, —CH₂—SO₂NR²³R²⁴, —C₂H₄—SO₂NR²³R²⁴, —C₃H₆—SO₂NR²³R²⁴, —SO₂NH₂, —CH₂—SO₂NH₂, —C₂H₄—SO₂NH₂, —C₃H₆—SO₂NH₂,

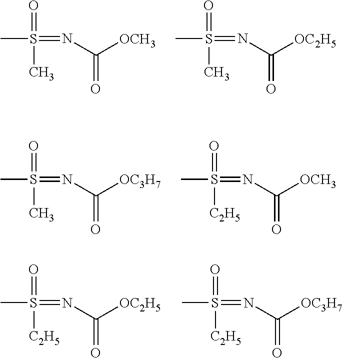

—CH(CH₃)—C₂H₅, —C(CH₃)₃, —C₅H₁₁, —CH₂—CH₂—CH₂—CH₂R⁶⁴, —O—CH₂—CH₂R⁶⁴, —CH₂R⁶⁴, —O—CH₂—CH₂—CH₂R⁶⁴, —CH₂—CH₂—CH₂R⁶⁴, —O—CH₂—CH₂—CH₂—CH₂R⁶⁴, —CH₂—CH₂R⁶⁴, —O—CH₂—CH₂—CH₂—CH₂—CH₂R⁶⁴, —CH₂—CH₂—CH₂—CH₂—CH₂R⁶⁴, —O—CH₂—CH₂—CH₂—CH₂—CH₂—CH₂R⁶⁴, —CH₂—CH₂—CH₂—CH₂—CH₂—CH₂R⁶⁴, —OCH₂Ph, —O—CH₂R⁶⁴, wherein R⁶⁴ represents -Ph, —F, —Cl, —Br or —I. Preferred are compounds wherein, R⁴ is selected from —NO₂, —NH₂, —CONH₂, —SO₂CH₃, —SO₂C₂H₅, —SO₂C₃H₇, —NH—SO₂—CH₃, —NH—SO₂—C₂H₅, —NH—SO₂—C₃H₇, —NHCO—CH₃, —NHCO—C₂H₅, —NHCO—C₃H₇, —SO₂NR²³R²⁴, —CH₂—SO₂NR²³R²⁴, —C₂H₄—SO₂NR²³R²⁴, —C₃H₆—SO₂NR²³R²⁴, —SO₂NH₂, —CH₂—SO₂NH₂, —C₂H₄—SO₂NH₂, —C₃H₆—SO₂NH₂,

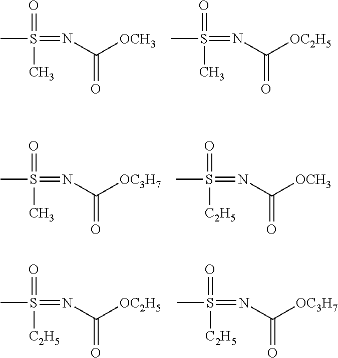

Moreover it is especially preferred that not both substituents -L-R³ and —R⁴ are hydrogen. Thus it is preferred that the phenyl substituent R¹ and the pyridyl substituent R¹ have at least one substituent and preferably one substituent in meta position and most preferably the preferred substituents mentioned above for -L-R³ and —R⁴ in meta position and especially preferred for —R⁴ in meta position. Consequently the following R¹ residues are preferred and especially preferred are the following substituents R¹ with the preferred substituents for -L-R³ and —R⁴:

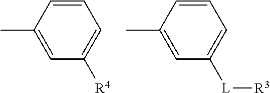

Also preferred are compounds of general formula (I), wherein R⁸³ is —H, —OH, —NO₂, —CN, —F, —Cl, —Br, —I, —NH₂, —NH(CH₃), —N(CH₃)₂, —NH(C₂H₅), —N(C₂H₅)₂, —CF₃, —CH₃, —C₂H₅, —C₃H₇, —CH(CH₃)₂, —C₄H₉, —C(CH₃)₃, —CH₂—NH₂, —CH₂—NH(CH₃), —CH₂—N(CH₃)₂, —CH₂—NH(C₂H₅), —CH₂—N(C₂H₅)₂, —CH₂—CH₂NH₂, —CH₂—CH₂—NH(CH₃), —CH₂—CH₂—N(CH₃)₂, —CH₂—CH₂—NH(C₂H₅), —CH₂—CH₂—N(C₂H₅)₂, —CH₂—CH₂—CH₂—NH₂, —CH₂—CH₂—CH₂—NH(CH₃), —CH₂—CH₂—CH₂—N(CH₃)₂, —CH₂—CH₂—CH₂—NH(C₂H₅), —CH₂—CH₂—CH₂—N(C₂H₅)₂, —O—CH₃, —O—CH₂—CH₃, —O—CH₂—CH₂—CH₃, —CHO, —CH₂OH, —CO—CH₃, —CO—CH₂—CH₃, —CO—CH₂—CH₂—CH₃, —CO—CH₂—CH₂—CH₂—CH₃, —CH₂O—CH₃, —CH₂O—CH₂—CH₃, —CH₂O—CH₂—CH₂—CH₃, —CH₂F, —CH₂Cl, —CH₂Br, —CH₂—CH₂F, —CH₂—CH₂Cl, —CH₂—CH₂Br, —CH₂—CH₂—CH₂F, —CH₂—CH₂—CH₂Cl, —CH₂—CH₂—CH₂Br.

Moreover compounds of general formula (I) are preferred, wherein B represents a bond, —CH₂—, —CH₂CH₂—, —CH₂CH₂CH₂—, —CH₂CH₂CH₂CH₂—, —CH₂CH₂CH₂CH₂CH₂— and/or wherein Y represents a bond, —O—, or —NH—.

In addition compounds of general formula (I) are preferred, wherein R⁸⁴ represents a bond, —CH₂—, —CH₂CH₂—, —CH₂CH₂CH₂—, —CH₂CH₂CH₂CH₂—, —CH₂CH₂CH₂CH₂CH₂—.

Preferred are also compounds of general formula (I), wherein R⁸⁵ is —H, —OH, —OCH₃, —OC₂H₅, —OC₃H₇, —O-cyclo-C₃H₅, —OCH(CH₃)₂, —OC(CH₃)₃, —OC₄H₉, -Ph, —OPh, —OCH₂-Ph, —OCPh₃, —NO₂, —F, —Cl, —Br, —I, —CN, —CHO, —COCH₃, —COC₂H₅, —COC₃H₇, —CO-cyclo-C₃H₅, —COCH(CH₃)₂, —COC(CH₃)₃, —COC₄H₉, —COOH, —COOCH₃, —COOC₂H₅, —COOC₃H₇, —COOC₄H₉, —COO-cyclo-C₃H₅, —COOCH(CH₃)₂, —COOC(CH₃)₃, —OOC—CH₃, —OOC—C₂H₅, —OOC—C₃H₇, —OOC—C₄H₉, —OOC-cyclo-C₃H₅, —OOC—CH(CH₃)₂, —OOC—C(CH₃)₃, —CONR^{23′}R^{24′}, —NHCOCH₃, —NHCOC₂H₅, —NHCOC₃H₇, —NHCO-cyclo-C₃H₅, —NHCO—CH(CH₃)₂, —NHCOC₄H₉, —NHCO—C(CH₃)₃, —NHCO—OCH₃, —NHCO—OC₂H₅, —NHCO—OC₃H₇, —NHCO—O-cyclo-C₃H₅, —NHCO—OC₄H₉, —NHCO—OCH(CH₃)₂, —NHCO—OC(CH₃)₃, —NHCO—OCH₂Ph, —NR²³R²⁴, —CF₃, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, SO₂C₄H₉, —SO₂C(CH₃)₃, —SO₃H, —SO₂NR^{23′}R^{24′}, —OCF₃, —OC₂F₅, —NH—CO—NH₂, —NH—CO—NHCH₃, —NH—CO—NHC₂H₅, —NH—CO—NHC₃H₇, —NH—CO—NHC₄H₉, —NH—CO—NH-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —O—CO—NH₂, —O—CO—NHCH₃, —O—CO—NHC₂H₅, —O—CO—NHC₃H₇, —O—CO—NHC₄H₉, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(C₄H₉)₂, —O—CO—N(cyclo-C₃H₅)₂, —O—CO—N[CH(CH₃)₂]₂, —O—CO—N[C(CH₃)₃]₂, 2-thienyl, 3-thienyl, 2-furanyl, 3-furanyl, 2-oxazolyl, 3-oxazolyl, 4-oxazolyl, 2-thiazolyl, 3-thiazolyl, 4-thiazolyl, 1-pyrazolyl, 3-pyrazolyl, 4-pyrazolyl, 5-pyrazolyl, 1-imidazolyl, 2-imidazolyl, 4-imidazolyl, 5-imidazolyl, phenyl, 1-naphthyl, 2-naphthyl, 2-pyridyl, 3-pyridyl, 4-pyridyl, 2-pyrimidinyl, 4-pyrimidinyl, 5-pyrimidinyl, 2-pyrazinyl, 3-pyridazinyl, 4-pyridazinyl, 1,3,5-triazin-2-yl,

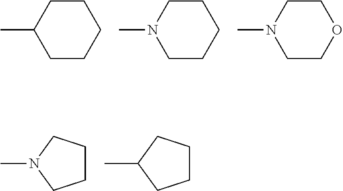

with the proviso that R⁸³ is not —H, if the group —B—Y—R⁸⁴—R⁸⁵ is hydrogen.

Also preferred are compounds of general formula (I), wherein R⁹⁸ is —NO₂, —CN, —F, —Cl, —Br, —I, —NH₂, —OH, —CF₃, —CH₃, —C₂H₅, —C₃H₇, —CH(CH₃)₂, —C₄H₉, —C(CH₃)₃, —CH₂—NH₂, —CH₂—NH(CH₃), —CH₂—N(CH₃)₂, —CH₂—NH(C₂H₅), —CH₂—N(C₂H₅)₂, —CH₂—CH₂—NH₂, —CH₂—CH₂—NH(CH₃), —CH₂—CH₂—N(CH₃)₂, —CH₂—CH₂—NH(C₂H₅), —CH₂—CH₂—N(C₂H₅)₂, —CH₂—CH₂—CH₂—NH₂, —CH₂—CH₂—CH₂—NH(CH₃), —CH₂—CH₂—CH₂—N(CH₃)₂, —CH₂—CH₂—CH₂—NH(C₂H₅), —CH₂—CH₂—CH₂—N(C₂H₅)₂, —O—CH₃, —O—CH₂—CH₃, —O—CH₂—CH₂—CH₃, —CH₂O—CH₃, —CH₂O—CH₂—CH₃, —CH₂O—CH₂—CH₂—CH₃, —CH₂F, —CH₂Cl, —CH₂Br, —CH₂—CH₂F, —CH₂—CH₂Cl, —CH₂—CH₂Br, —CH₂—CH₂—CH₂F, —CH₂—CH₂—CH₂Cl, —CH₂—CH₂—CH₂Br, —OCH₂Ph, —OCH₂—CH₂-Ph, —CH₂—O—CH₂-Ph.

Moreover especially preferred are compounds of the general formula (I), wherein

R¹ is

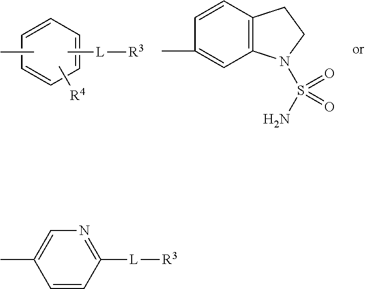

L is a bond, —CH₂—, or —CH₂CH₂—;

R³ is —H, —SO₂NR²³R²⁴, —CONR²³R²⁴, —NO₂, —NH₂, —NHSO₂R²², —NHCOR²², —SO₂R²², —NH—CO—NH-Ph, or -Ph,

R⁴ is —H, —CH₂—SO₂NR²³R²⁴, —SO₂NR²³R²⁴, —CONH₂, —C₂H₄—SO₂NR²³R²⁴, —NH—SO₂—CH₃, —NH—SO₂—C₂H₅, —NH—SO₂—C₃H₇, —NHCO—CH₃, —NHCO—C₂H₅, —NO₂, —NH₂, —SO₂CH₃, or

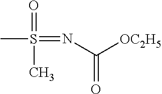

R²³ and R²⁴ are independently selected from —H, —CH₃, —C₂H₅, —C₃H₇, -(cyclo-C₃H₅), —CH₂—CH₂—CH₂—CH₂—NH₂, or —CH₂—CH₂—CH₂—CH₂—NH—COOC(CH₃)₃,

R² represents

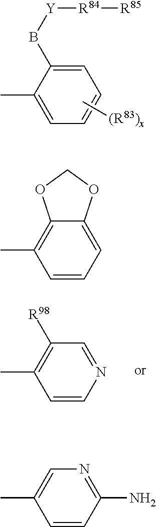

B is a bond or CH₂;

Y is a bond, —O—, or —NH—;

R⁸³ is selected from —H, CN, F, Cl, —O—CR⁶²R⁶³R⁶⁴, —CF₃, —CH₂OR^{23′}, CR^{23′}O, —CR⁶²R⁶³NR^{23′}R^{24′}, —CR⁶²R⁶³R⁶⁴;

R^{23′} and R^{24′} represent independently of each other —H, —CH₃, (cyclo-C₃H₅);

R⁶²R⁶⁴ represent independently of each other —H, —CH₃, -Ph, —F, (cyclo-C₃H₅);

R⁸⁴ is selected from a bond, —CH₂, or —CH₂—CH₂—CH₂—CH₂—;

R⁸⁵ is selected from —H, —CF₃, —OCH₃, —OCH(CH₃)₂, —CN, —NHCOCH₃, —OCH₂-(cyclo-C₃H₅), —NR²³R²⁴, -Ph, —OPh, —NHCO—OC(CH₃)₃,

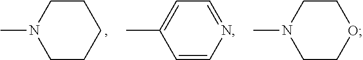

R⁹⁸ represents OCH₃;

and salts, solvates or salts of solvates of the afore-mentioned compounds and especially the hydrochloride salt or the trifluoroacetate salt of these compounds.

Moreover especially preferred are compounds of the general formula (I), wherein

R¹ is

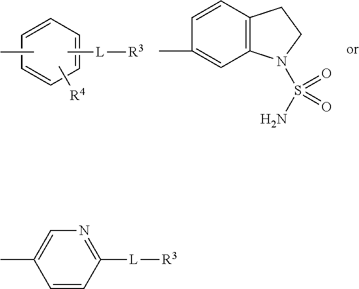

L is a bond, —CH₂, or —CH₂CH₂—;

R³ is —H, —SO₂NH₂, —CONH₂, —NO₂, —NH₂, NH—SO₂—CH₃, —NH—SO₂—C₃H₇, —NHCO—CH₃, —SO₂CH₃, -Ph, —SO₂NH—CH₂—CH₂—CH₂—CH₂NH—COOC(CH₃)₃, —NH—CO—NH-Ph, or —SO₂—NH—CH₂—CH₂—CH₂—CH₂—NH₂,

R⁴ is —H, —CH₂—SO₂NH₂, —SO₂NH₂, —C₂H₄—SO₂NH₂, —CONH₂, —NH—SO₂—CH₃, —NH—SO₂—C₃H₇, —NHCO—CH₃, —NO₂, —NH₂, —SO₂CH₃, or

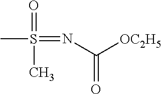

R² represents

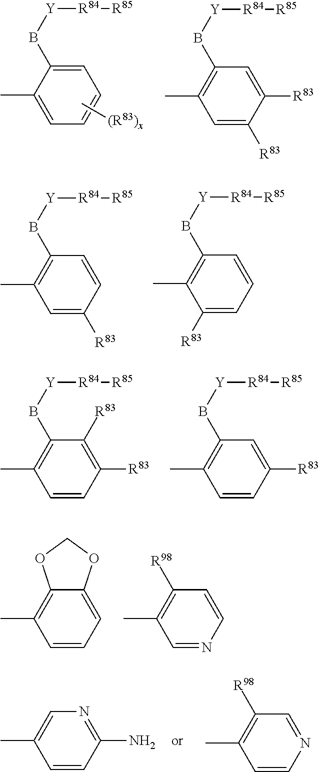

B is a bond or —CH₂—;

Y is a bond, —O—, or —NH—;

R⁸³ is selected from —H, —F, —Cl, —O—CH₃, —O—C₂H₅, —OCH₂-(cyclo-C₃H₅), —CN, —CF₃, —CH₂OH, —CHO, —CH₂—NH(cyclo-C₃H₅), —CH₂—NH(CH₃), —CF₃;

R⁸⁴ is selected from a bond, —CH₂—, or —CH₂—CH₂—CH₂CH₂;

R⁸⁵ is selected from —H, —CF₃, —OCH₃, —OCH(CH₃)₂, —CN, —NHCOCH₃, —OCH₂-(cyclo-C₃H₅), —NH₂, —NH-(cyclo-C₃H₅), -Ph, —OPh, NHCO—OC(CH₃)₃,

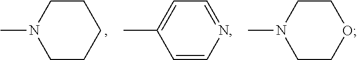

R⁹⁸ represents OCH₃;

and salts, solvates or salts of solvates of the afore-mentioned compounds and especially the hydrochloride salt or the trifluoroacetate salt of these compounds.

In a particularly preferred embodiment the present invention concerns compounds of formula (I), wherein

R¹ represents

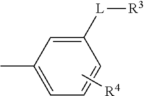

in which

the substituent -L-R³ is —SO₂NH₂ or —CH₂SO₂NH₂,

R⁴ is H;

R² represents 2-methoxyphenyl, 4-fluoro-2-methoxyphenyl or 2-benzyloxyphenyl,

or their salts, solvates or salts of solvates and especially the hydrochloride salt or the trifluoroacetate salt.

In another particularly preferred embodiment the present invention concerns compounds of formula (I) selected from
3-[(4-(2-Methoxyphenyl)-1,3,5-triazin-2-yl)amino]benzenemethanesulfonamide (B1),
3-[(4-(2-Methoxyphenyl)-1,3,5-triazin-2-yl)amino]benzenesulfonamide (C1),
3-[(4-(4-Fluoro-2-methoxyphenyl)-1,3,5-triazin-2-yl)amino]benzenemethanesulfonamide (B2),
3-[(4-(2-Benzyloxyphenyl)-1,3,5-triazin-2-yl)amino]benzenemethanesulfonamide (B 13),
or their salts, solvates or salts of solvates and especially the hydrochloride salt or the trifluoroacetate salt.

In another particularly preferred embodiment the present invention concerns 3-[(4-(4-Fluoro-2-methoxyphenyl)-1,3,5-triazin-2-yl)amino]benzenemethanesulfonamide, or its salts, solvates or salts of solvates and especially the hydrochloride salt or the trifluoroacetate salt.

In another particularly preferred embodiment the present invention concerns 1-(3-{[4-(4-fluoro-2-methoxyphenyl)-1,3,5-triazin-2-yl]amino}phenyl)methanesulfonamide hydrochloride.

Excluded are these compounds from the present invention, wherein —R⁴ and -L-R³ are methoxy or ethoxy groups.

Excluded from the present invention are also compounds, wherein R¹ is

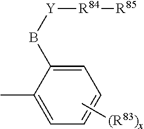

and wherein R² is

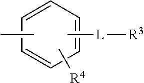

and wherein one of

—R⁴ and -L-R³ is a chloro substituent and wherein one of B—Y—R⁸⁴R⁸⁵ and —R⁸³ is also a chloro substituent. More general, compounds of general formula (I) with two or more chloro substituents are not preferred and might be excluded.

If the group B—Y—R⁸⁴—R⁸⁵ represents the substituent —NH—CO-Ph, the phenyl moiety R¹ has at least one substituent which is not in para position to the bond between the phenyl moiety R¹ and the triazine ring or the substituent -L-R³, wherein L is a bond is different from the substituent —CO—NH₂. In addition the following compound is excluded from the scope of the present invention by disclaimer:

4-[4-(2-benzoylaminophenyl)-[1,3,5]triazin-2-ylamino]benzamide

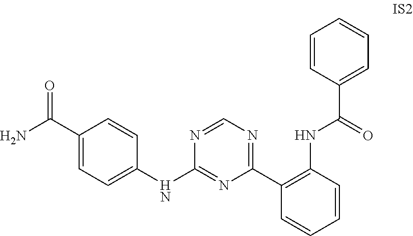 [SMILES: NC(=O)c1ccc(Nc2ncnc(-c3ccccc3NC(=O)c3ccccc3)n2)cc1]

In a further aspect of the present invention, the novel compounds according to the general formula (I) represent chiral compounds. The novel compounds according to the general formula (I) represent a racemate, or a S or a R enantiomer or a mixture of isomers.

In yet another preferred embodiment of the present invention, the compound according to the general formula (I) is selected from the group of compounds depicted in the following Table 1.

TABLE 1

| Compound No. | Structure | Nomenclature |
|---|---|---|
| B1 | 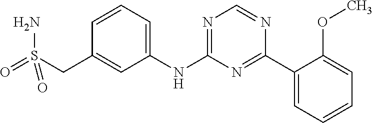 [SMILES: COc1ccccc1-c1ncnc(Nc2cccc(CS(N)(=O)=O)c2)n1] | 3-[(4-(2-Methoxyphenyl)-1,3,5-triazin- 2- yl)amino]benzenemethanesulfonamide |
| B2 | 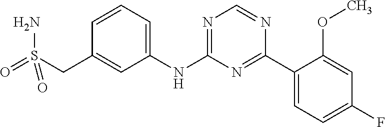 [SMILES: COc1cc(F)ccc1-c1ncnc(Nc2cccc(CS(N)(=O)=O)c2)n1] | 3-[(4-(4-Fluoro-2-methoxyphenyl)- 1,3,5-triazin-2-yl)amino] benzenemethanesulfonamide |
| B3 | 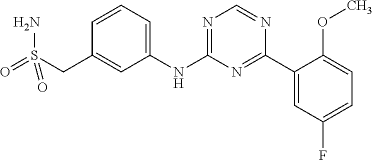 [SMILES: COc1ccc(F)cc1-c1ncnc(Nc2cccc(CS(N)(=O)=O)c2)n1] | 3-[(4-(5-Fluoro-2-methoxyphenyl)- 1,3,5-triazin-2-yl)amino] benzenemethanesulfonamide |
| B4 | 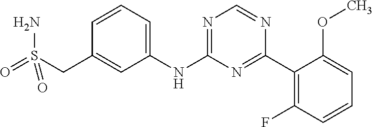 [SMILES: COc1cccc(F)c1-c1ncnc(Nc2cccc(CS(N)(=O)=O)c2)n1] | 3-[(4-(6-Fluoro-2-methoxyphenyl)- 1,3,5-triazin-2-yl)amino] benzenemethanesulfonamide |
| B5 | 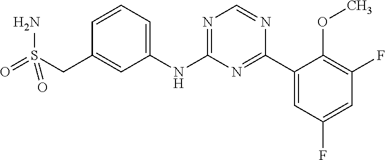 [SMILES: COc1c(F)cc(F)cc1-c1ncnc(Nc2cccc(CS(N)(=O)=O)c2)n1] | 3-[(4-(3,5-Difluoro-2-methoxyphenyl)- 1,3,5-triazin-2-yl)amino] benzenemethanesulfonamide |
| B6 | 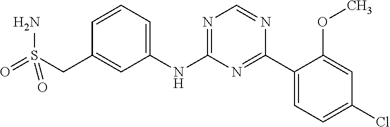 [SMILES: COc1cc(Cl)ccc1-c1ncnc(Nc2cccc(CS(N)(=O)=O)c2)n1] | 3-[(4-(4-Chloro-2-methoxyphenyl)- 1,3,5-triazin-2-yl)amino] benzenemethanesulfonamide |
| B7 | 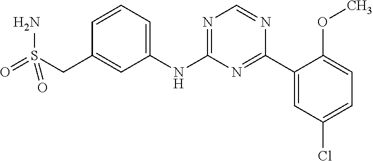 [SMILES: COc1ccc(Cl)cc1-c1ncnc(Nc2cccc(CS(N)(=O)=O)c2)n1] | 3-[(4-(5-Chloro-2-methoxyphenyl)- 1,3,5-triazin-2-yl)amino] benzenemethanesulfonamide |
| B8 | 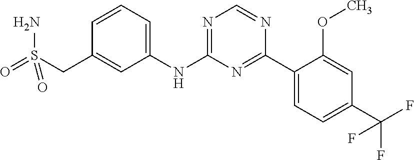 [SMILES: COc1cc(C(F)(F)F)ccc1-c1ncnc(Nc2cccc(CS(N)(=O)=O)c2)n1] | 3-[(4-(2-Methoxy-4-trifluoromethyl- phenyl)-1,3,5-triazin-2-yl)amino] enzenemethanesulfonamide |
| B9 | 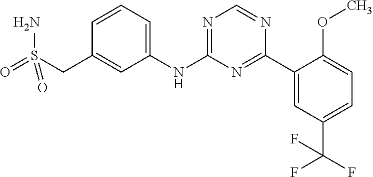 [SMILES: COc1ccc(C(F)(F)F)cc1-c1ncnc(Nc2cccc(CS(N)(=O)=O)c2)n1] | 3-[(4-(2-Methoxy-5-trifluoromethyl- phenyl)-1,3,5-triazin-2-yl)amino] benzenemethanesulfonamide |
| B10 | 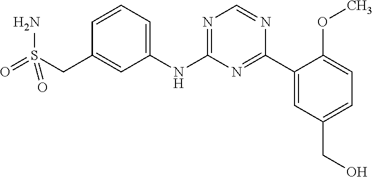 [SMILES: COc1ccc(CO)cc1-c1ncnc(Nc2cccc(CS(N)(=O)=O)c2)n1] | 3-[(4-(5-Hydroxymethyl-2- methoxyphenyl)-1,3,5-triazin-2- yl)amino] benzenemethanesulfonamide |
| B11 | 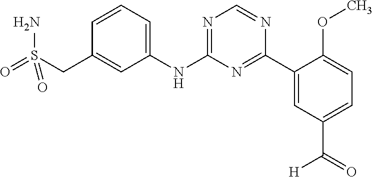 [SMILES: COc1ccc(C=O)cc1-c1ncnc(Nc2cccc(CS(N)(=O)=O)c2)n1] | 3-[(4-(5-Formyl-2-methoxyphenyl)- 1,3,5-triazin-2-yl)amino] benzenemethanesulfonamide |
| B12 | 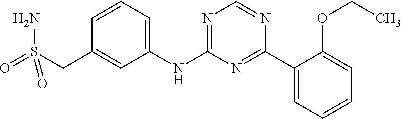 [SMILES: CCOc1ccccc1-c1ncnc(Nc2cccc(CS(N)(=O)=O)c2)n1] | 3-[(4-(2-Ethoxyphenyl)-1,3,5-triazin-2- yl)amino]benzenemethanesulfonamide |
| B13 | 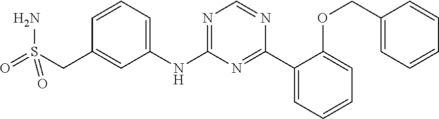 [SMILES: NS(=O)(=O)Cc1cccc(Nc2ncnc(-c3ccccc3OCc3ccccc3)n2)c1] | 3-[(4-(2-Benzyloxyphenyl)-1,3,5-triazin- 2-yl)amino] benzenemethanesulfonamide |
| B14 | 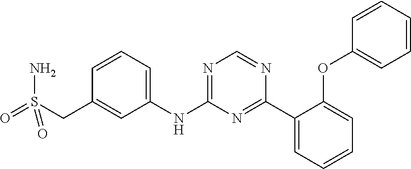 [SMILES: NS(=O)(=O)Cc1cccc(Nc2ncnc(-c3ccccc3Oc3ccccc3)n2)c1] | 1-(3-{[4-(2-phenoxyphenyl)-1,3,5- triazin-2- yl]amino}phenyl)methanesulfonamide |
| B15 | 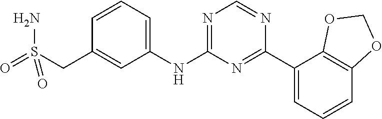 [SMILES: NS(=O)(=O)Cc1cccc(Nc2ncnc(-c3cccc4c3OCO4)n2)c1] | 3-[(4-(1,3-Benzodioxo1-4-yl)-1,3,5- triazin-2- yl)amino]benzenemethanesulfonamide |
| B16 | 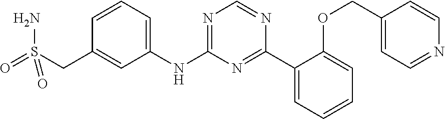 [SMILES: NS(=O)(=O)Cc1cccc(Nc2ncnc(-c3ccccc3OCc3ccncc3)n2)c1] | 3-[(4-(2-((4-Pyridinyl)methoxy)phenyl)- 1,3,5-triazin-2- yl)amino]benzenemethanesulfonamide |
| B17 | 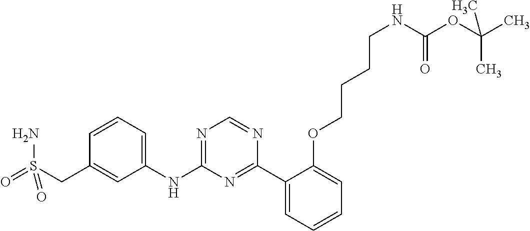 [SMILES: CC(C)(C)OC(=O)NCCCCOc1ccccc1-c1ncnc(Nc2cccc(CS(N)(=O)=O)c2)n1] | 3-[(4-(2-(4-(tert- Butoxycarbonylamino)butoxy)phenyl)- 1,3,5-triazin-2- yl)amino]benzenemethanesulfonamide |
| B18 | 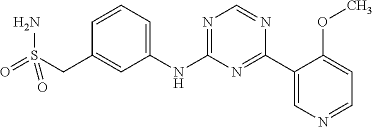 [SMILES: COc1ccncc1-c1ncnc(Nc2cccc(CS(N)(=O)=O)c2)n1] | 3-[(4-(4-Methoxypyridin-3-yl)-1,3,5- triazin-2- yl)amino]benzenemethanesulfonamide |
| B19 | 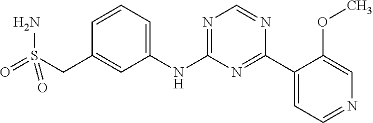 [SMILES: COc1cnccc1-c1ncnc(Nc2cccc(CS(N)(=O)=O)c2)n1] | 3-[(4-(3-Methoxypyridin-4-yl)-1,3,5- triazin-2- yl)amino]benzenemethanesulfonamide |
| B20 | 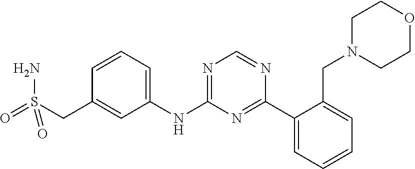 [SMILES: NS(=O)(=O)Cc1cccc(Nc2ncnc(-c3ccccc3CN3CCOCC3)n2)c1] | 3-[(4-(2-((Morpholin-4- yl)methyl)phenyl)-1,3,5-triazin-2- yl)amino]benzenemethanesulfonamide |
| B21 | 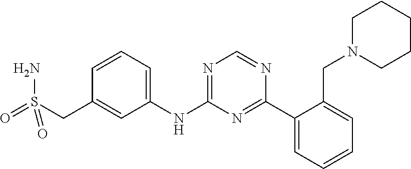 [SMILES: NS(=O)(=O)Cc1cccc(Nc2ncnc(-c3ccccc3CN3CCCCC3)n2)c1] | 3-[(4-(2-((Piperidin-1-yl)methyl)phenyl)- 1,3,5-triazin-2- yl)amino]benzenemethanesulfonamide |
| B22 | 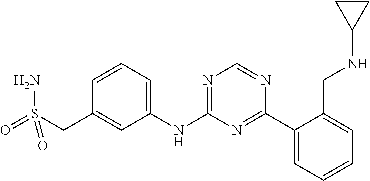 [SMILES: NS(=O)(=O)Cc1cccc(Nc2ncnc(-c3ccccc3CNC3CC3)n2)c1] | 3-[(4-(2-(Cyclopropylamino- methyl)phenyl)-1,3,5-triazin-2- yl)amino]benzenemethanesulfonamide |
| B23 | 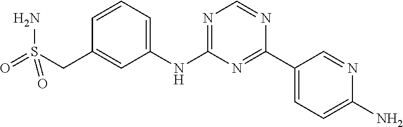 [SMILES: Nc1ccc(-c2ncnc(Nc3cccc(CS(N)(=O)=O)c3)n2)cn1] | 3-[(4-(6-Aminopyridin-3-yl)-1,3,5- triazin-2- yl)amino]benzenemethanesulfonamide |
| B24 | 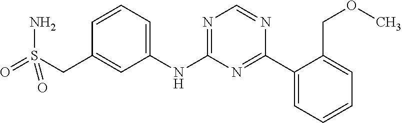 [SMILES: COCc1ccccc1-c1ncnc(Nc2cccc(CS(N)(=O)=O)c2)n1] | 3-[(4-(2-(Methoxymethyl)phenyl)-1,3,5- triazin-2- yl)amino]benzenemethanesulfonamide |
| C1 | 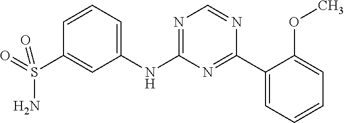 [SMILES: COc1ccccc1-c1ncnc(Nc2cccc(S(N)(=O)=O)c2)n1] | 3-[(4-(2-Methoxyphenyl)-1,3,5-triazin- 2-yl)amino]benzenesulfonamide |
| D1 | 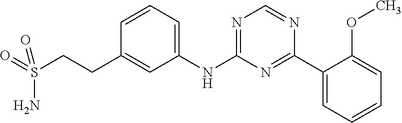 [SMILES: COc1ccccc1-c1ncnc(Nc2cccc(CCS(N)(=O)=O)c2)n1] | 2-[3-((4-(2-Methoxyphenyl)-1,3,5- triazin-2- yl)amino)phenyl]ethanesulfonamide |
| D2 | 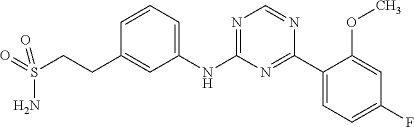 [SMILES: COc1cc(F)ccc1-c1ncnc(Nc2cccc(CCS(N)(=O)=O)c2)n1] | 2-[3-((4-(4-Fluoro-2-methoxyphenyl)- 1,3,5-triazin-2- yl)amino)phenyl]ethanesulfonamide |
| E1 | 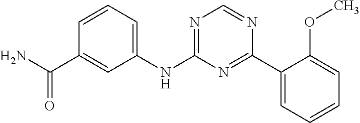 [SMILES: COc1ccccc1-c1ncnc(Nc2cccc(C(N)=O)c2)n1] | 3-[(4-(2-Methoxyphenyl)-1,3,5-triazin- 2-yl)amino]benzamide |
| F1 | 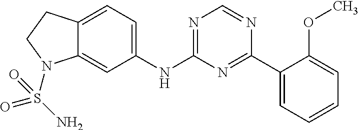 [SMILES: COc1ccccc1-c1ncnc(Nc2ccc3c(c2)N(S(N)(=O)=O)CC3)n1] | 6-[(4-(2-Methoxyphenyl)-1,3,5-triazin- 2-yl)amino]-2,3-dihydro-1H-indole-1- sulfonamide |
| G1 | 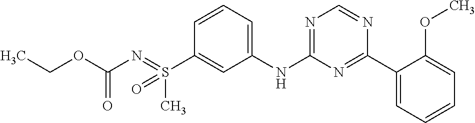 [SMILES: CCOC(=O)N=S(C)(=O)c1cccc(Nc2ncnc(-c3ccccc3OC)n2)c1] | rac-S-[3-((4-(2-Methoxyphenyl)-1,3,5- triazin-2-yl)amino)phenyl]-N- ethoxycarbonyl-S-methyl-sulfoximide |
| H1 | 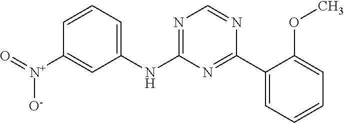 [SMILES: COc1ccccc1-c1ncnc(Nc2cccc([N+](=O)[O-])c2)n1] | 4-(2-Methoxyphenyl)-N-(3-nitrophenyl)- 1,3,5-triazine-2-amine |
| I1 | 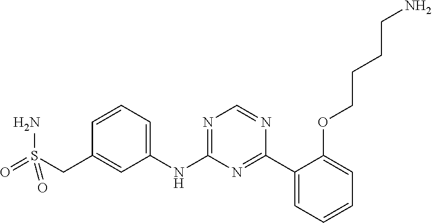 [SMILES: NCCCCOc1ccccc1-c1ncnc(Nc2cccc(CS(N)(=O)=O)c2)n1] | 3-[(4-(2-(4-Aminobutoxy)phenyl)-1,3,5- triazin-2- yl)amino]benzenemethanesulfonamide |
| J1 | 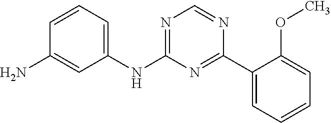 [SMILES: COc1ccccc1-c1ncnc(Nc2cccc(N)c2)n1] | N-(3-Aminophenyl)-4-(2- methoxyphenyl)-1,3,5-triazine-2-amine |
| K1 | 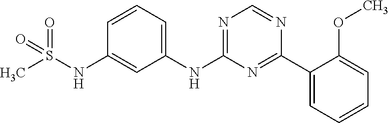 | N-[3-((4-(2-Methoxyphenyl)-1,3,5- triazin-2-yl)amino)phenyl]- methanesulfonamide |
| L1 | 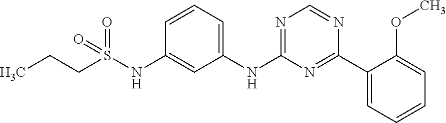 [SMILES: CCCS(=O)(=O)Nc1cccc(Nc2ncnc(-c3ccccc3OC)n2)c1] | N-[3-((4-(2-Methoxyphenyl)-1,3,5- triazin-2-yl)amino)phenyl]- propanesulfonamide |
| M1 | 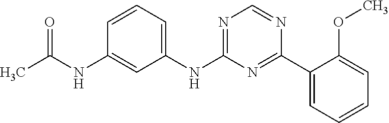 [SMILES: COc1ccccc1-c1ncnc(Nc2cccc(NC(C)=O)c2)n1] | N-[3-((4-(2-Methoxyphenyl)-1,3,5- triazin-2-yl)amino)phenyl]acetamide |
| N1 | 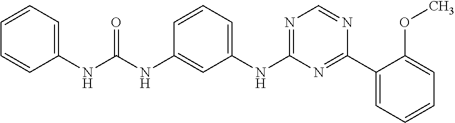 [SMILES: COc1ccccc1-c1ncnc(Nc2cccc(NC(=O)Nc3ccccc3)c2)n1] | N-[3-((4-(2-Methoxyphenyl)-1,3,5- triazin-2-yl)amino)phenyl]-N′-phenyl- urea |
| O1 | 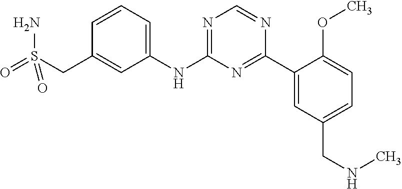 [SMILES: CNCc1ccc(OC)c(-c2ncnc(Nc3cccc(CS(N)(=O)=O)c3)n2)c1] | 3-[(4-(2-Methoxy-5-(methylamino- methyl)phenyl)-1,3,5-triazin-2- yl)amino]benzenemethanesulfonamide |
| P1 | 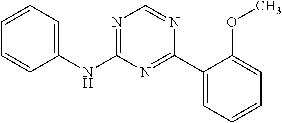 [SMILES: COc1ccccc1-c1ncnc(Nc2ccccc2)n1] | 4-(2-Methoxyphenyl)-N-phenyl-1,3,5- triazine-2-amine |
| Q1 | 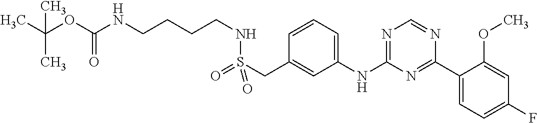 [SMILES: COc1cc(F)ccc1-c1ncnc(Nc2cccc(CS(=O)(=O)NCCCCNC(=O)OC(C)(C)C)c2)n1] | tert-Butyl [4-((3-((4-(4-Fluoro-2- methoxyphenyl)-1,3,5-triazin-2- yl)amino)phenyl)methylsulfonamido) butyl]carbamate |
| R1 | 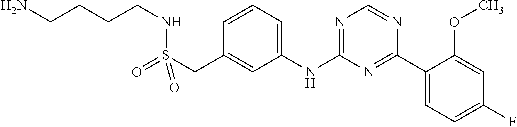 | N-(4-Aminobutyl)-1-[3-((4-(4-fluoro-2- methoxyphenyl)-1,3,5-triazin-2- yl)amino)phenyl]methanesulfonamide |
| S1 | 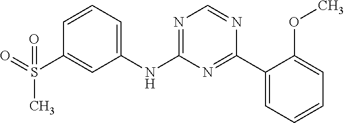 [SMILES: COc1ccccc1-c1ncnc(Nc2cccc(S(C)(=O)=O)c2)n1] | 4-(2-Methoxyphenyl)-N-(3- (methylsulfonyl)phenyl)-1,3,5-triazin-2- amine |
| T1 | 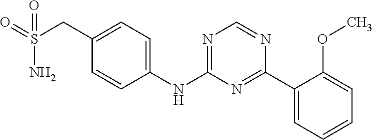 [SMILES: COc1ccccc1-c1ncnc(Nc2ccc(CS(N)(=O)=O)cc2)n1] | 4-[(4-(2-Methoxyphenyl)-1,3,5-triazin- 2-yl)amino]benzenemethane- sulfonamide |
| U1 | 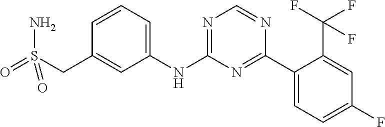 [SMILES: NS(=O)(=O)Cc1cccc(Nc2ncnc(-c3ccc(F)cc3C(F)(F)F)n2)c1] | 1-[3-({4-[4-fluoro-2- (trifluoromethyl)phenyl]-1,3,5-triazin-2- yl}amino)phenyl]methanesulfonamide |
| U2 | 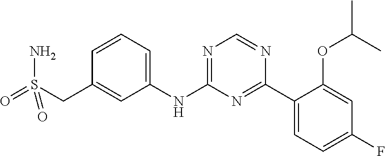 [SMILES: CC(C)Oc1cc(F)ccc1-c1ncnc(Nc2cccc(CS(N)(=O)=O)c2)n1] | 1-[3-({4-[4-fluoro-2-(propan-2- yloxy)phenyl]-1,3,5-triazin-2- yl}amino)phenyl]methanesulfonamide |
| U3 | 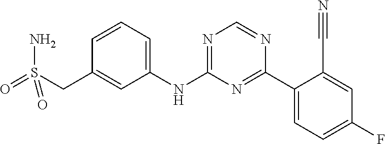 [SMILES: N#Cc1cc(F)ccc1-c1ncnc(Nc2cccc(CS(N)(=O)=O)c2)n1] | 1-(3-{[4-(2-cyano-4-fluorophenyl)- 1,3,5-triazin-2- yl]amino}phenyl)methanesulfonamide |
| U4 | 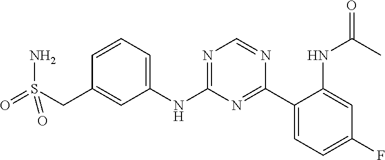 [SMILES: CC(=O)Nc1cc(F)ccc1-c1ncnc(Nc2cccc(CS(N)(=O)=O)c2)n1] | N-[5-fluoro-2-(4-{[3- (sulfamoylmethyl)phenyl]amino}-1,3,5- triazin-2-yl)phenyl]acetamide |
| U5 | 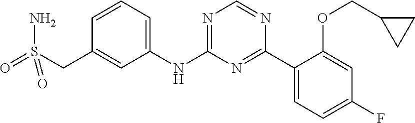 [SMILES: NS(=O)(=O)Cc1cccc(Nc2ncnc(-c3ccc(F)cc3OCC3CC3)n2)c1] | 1-[3-({4-[2-(cyclopropylmethoxy)-4- fluorophenyl]-1,3,5-triazin-2- yl}amino)phenyl]methanesulfonamide |
| U6 | 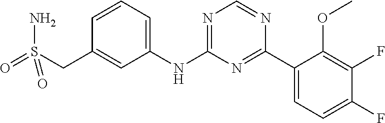 [SMILES: COc1c(-c2ncnc(Nc3cccc(CS(N)(=O)=O)c3)n2)ccc(F)c1F] | 1-(3-{[4-(3,4-difluoro-2- methoxyphenyl)-1,3,5-triazin-2- yl]amino}phenyl)methanesulfonamide |
| U7 | 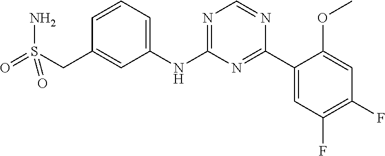 [SMILES: COc1cc(F)c(F)cc1-c1ncnc(Nc2cccc(CS(N)(=O)=O)c2)n1] | 1-(3-{[4-(4,5-difluoro-2- methoxyphenyl)-1,3,5-triazin-2- yl]amino}phenyl)methanesulfonamide |
| U8 | 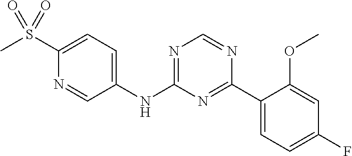 [SMILES: COc1cc(F)ccc1-c1ncnc(Nc2ccc(S(C)(=O)=O)nc2)n1] | 4-(4-fluoro-2-methoxyphenyl)-N-[6- (methylsulfonyl)pyridin-3-yl]-1,3,5- triazin-2-amine |
| B1′ | 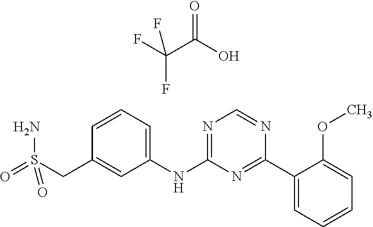 | 3-[(4-(2-Methoxyphenyl)-1,3,5-triazin- 2- yl)amino]benzenemethanesulfonamide trifluoroacetic acid salt |
| B2′ | 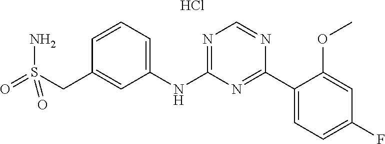 [SMILES: Cl ; COc1cc(F)ccc1-c1ncnc(Nc2cccc(CS(N)(=O)=O)c2)n1] | 1-(3-{[4-(4-fluoro-2-methoxyphenyl)- 1,3,5-triazin-2- yl]amino}phenyl)methanesulfonamide hydrochloride |
| B13′ | 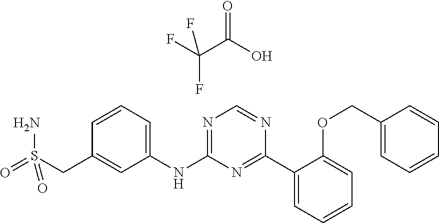 | 3-[(4-(2-Benzyloxyphenyl)-1,3,5-triazin- 2-yl)amino]benzenemethanesulfon- amide trifluoroacetic acid salt |
| C1′ | 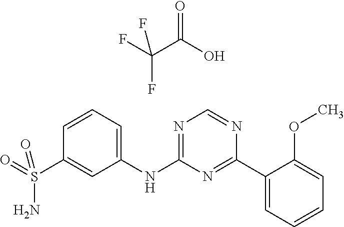 | 3-[(4-(2-Methoxyphenyl)-1,3,5-triazin- 2-yl)amino]benzenesulfonamide trifluoroacetic acid salt |
| B2″ | 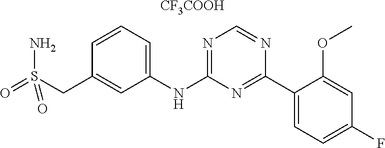 [SMILES: COc1cc(F)ccc1-c1ncnc(Nc2cccc(CS(N)(=O)=O)c2)n1 ; "CF3COOH"] | 1-(3-{[4-(4-fluoro-2-methoxyphenyl)- 1,3,5-triazin-2- yl]amino}phenyl)methanesulfonamide trifluoroacetic acid salt |

It is particularly preferred to use the following selective CDK9 inhibitors shown in Table 2 in accordance with the present invention:

TABLE 2

| Compound No. | Structure | Nomenclature |
|---|---|---|
| B1 | 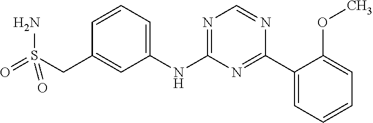 [SMILES: COc1ccccc1-c1ncnc(Nc2cccc(CS(N)(=O)=O)c2)n1] | 3-[(4-(2-Methoxyphenyl)-1,3,5-triazin- 2- yl)amino]benzenemethanesulfonamide |
| B2 | 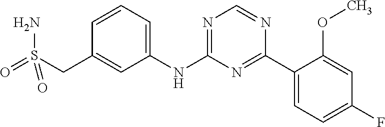 [SMILES: COc1cc(F)ccc1-c1ncnc(Nc2cccc(CS(N)(=O)=O)c2)n1] | 3-[(4-(4-Fluoro-2-methoxyphenyl)- 1,3,5-triazin-2-yl)amino] benzenemethanesulfonamide |
| B3 | 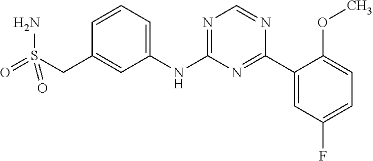 [SMILES: COc1ccc(F)cc1-c1ncnc(Nc2cccc(CS(N)(=O)=O)c2)n1] | 3-[(4-(5-Fluoro-2-methoxyphenyl)- 1,3,5-triazin-2-yl)amino] benzenemethanesulfonamide |
| B4 | 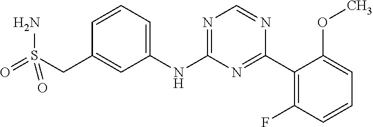 [SMILES: COc1cccc(F)c1-c1ncnc(Nc2cccc(CS(N)(=O)=O)c2)n1] | 3-[(4-(6-Fluoro-2-methoxyphenyl)- 1,3,5-triazin-2-yl)amino] benzenemethanesulfonamide |
| B6 | 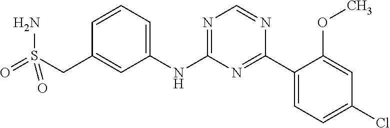 [SMILES: COc1cc(Cl)ccc1-c1ncnc(Nc2cccc(CS(N)(=O)=O)c2)n1] | 3-[(4-(4-Chloro-2-methoxyphenyl)- 1,3,5-triazin-2-yl)amino] benzenemethanesulfonamide |
| B7 | 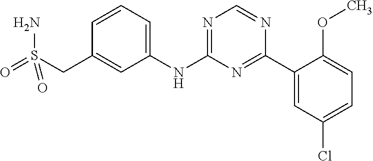 [SMILES: COc1ccc(Cl)cc1-c1ncnc(Nc2cccc(CS(N)(=O)=O)c2)n1] | 3-[(4-(5-Chloro-2-methoxyphenyl)- 1,3,5-triazin-2-yl)amino] benzenemethanesulfonamide |
| B12 | 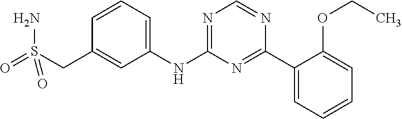 [SMILES: CCOc1ccccc1-c1ncnc(Nc2cccc(CS(N)(=O)=O)c2)n1] | 3-[(4-(2-Ethoxyphenyl)-1,3,5-triazin-2- yl)amino]benzenemethanesulfonamide |
| B13 | 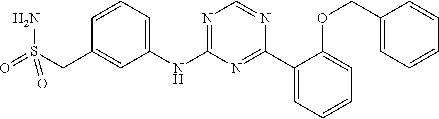 [SMILES: NS(=O)(=O)Cc1cccc(Nc2ncnc(-c3ccccc3OCc3ccccc3)n2)c1] | 3-[(4-(2-Benzyloxyphenyl)-1,3,5-triazin- 2-yl)amino] benzenemethanesulfonamide |
| B14 | 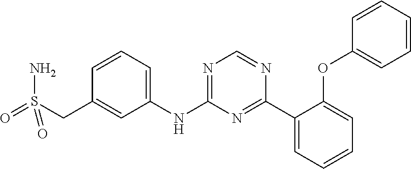 [SMILES: NS(=O)(=O)Cc1cccc(Nc2ncnc(-c3ccccc3Oc3ccccc3)n2)c1] | 1-(3-{[4-(2-phenoxyphenyl)-1,3,5- triazin-2- yl]amino}phenyl)methanesulfonamide |
| B16 | 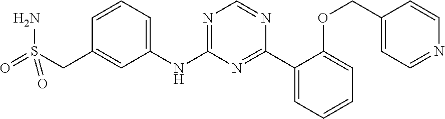 [SMILES: NS(=O)(=O)Cc1cccc(Nc2ncnc(-c3ccccc3OCc3ccncc3)n2)c1] | 3-[(4-(2-((4-Pyridinyl)methoxy)phenyl)- 1,3,5-triazin-2- yl)amino]benzenemethanesulfonamide |
| B17 | 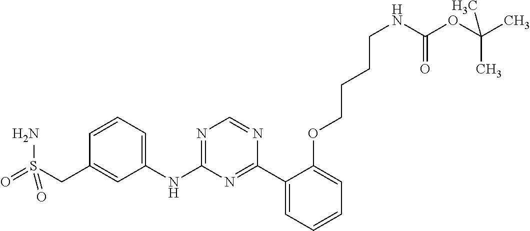 [SMILES: CC(C)(C)OC(=O)NCCCCOc1ccccc1-c1ncnc(Nc2cccc(CS(N)(=O)=O)c2)n1] | 3-[(4-(2-(4-(tert- Butoxycarbonylamino)butoxy)phenyl)- 1,3,5-triazin-2- yl)amino]benzenemethanesulfonamide |
| B18 | 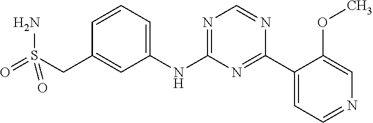 [SMILES: COc1cnccc1-c1ncnc(Nc2cccc(CS(N)(=O)=O)c2)n1] | 3-[(4-(3-Methoxypyridin-4-yl)-1,3,5- triazin-2- yl)amino]benzenemethanesulfonamide |
| B23 | 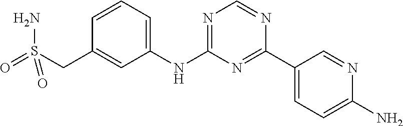 [SMILES: Nc1ccc(-c2ncnc(Nc3cccc(CS(N)(=O)=O)c3)n2)cn1] | 3-[(4-(6-Aminopyridin-3-yl)-1,3,5- triazin-2- yl)amino]benzenemethanesulfonamide |
| B24 | 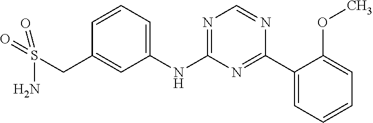 [SMILES: COc1ccccc1-c1ncnc(Nc2cccc(CS(N)(=O)=O)c2)n1] | 3-[(4-(2-Methoxyphenyl)-1,3,5-triazin- 2-yl)amino]benzenesulfonamide |
| C1 | 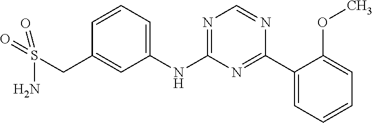 [SMILES: COc1ccccc1-c1ncnc(Nc2cccc(CS(N)(=O)=O)c2)n1] | 2-[3-((4-(2-Methoxyphenyl)-1,3,5- triazin-2- yl)amino)phenyl]ethanesulfonamide |
| D1 | 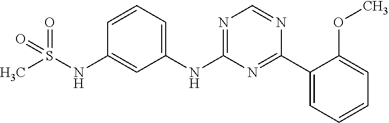 [SMILES: COc1ccccc1-c1ncnc(Nc2cccc(NS(C)(=O)=O)c2)n1] | N-[3-((4-(2-Methoxyphenyl)-1,3,5- triazin-2-yl)amino)phenyl]- methanesulfonamide |
| L1 | 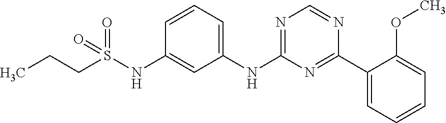 [SMILES: CCCS(=O)(=O)Nc1cccc(Nc2ncnc(-c3ccccc3OC)n2)c1] | N-[3-((4-(2-Methoxyphenyl)-1,3,5- triazin-2-yl)amino)phenyl]- propanesulfonamide |
| Q1 | 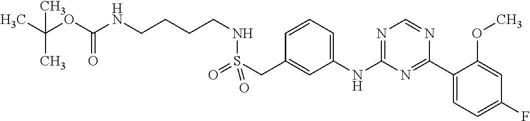 [SMILES: COc1cc(F)ccc1-c1ncnc(Nc2cccc(CS(=O)(=O)NCCCCNC(=O)OC(C)(C)C)c2)n1] | tert-Butyl [4-((3-((4-(4-Fluoro-2- methoxyphenyl)-1,3,5-triazin-2- yl)amino)phenyl)methylsulfonamido) butyl]carbamate |
| R1 | 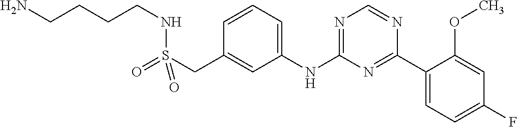 | N-(4-Aminobutyl)-1-[3-((4-(4-fluoro-2- methoxyphenyl)-1,3,5-triazin-2- yl)amino)phenyl]methanesulfonamide |
| S1 | 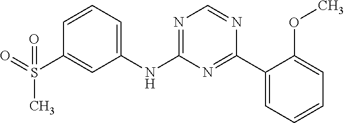 [SMILES: COc1ccccc1-c1ncnc(Nc2cccc(S(C)(=O)=O)c2)n1] | 4-(2-Methoxyphenyl)-N-(3- (methylsulfonyl)phenyl)-1,3,5-triazin-2- amine |
| U2 | 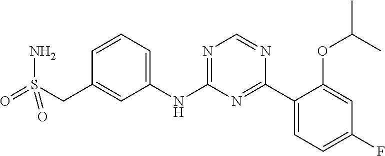 [SMILES: CC(C)Oc1cc(F)ccc1-c1ncnc(Nc2cccc(CS(N)(=O)=O)c2)n1] | 1-[3-({4-[4-Fluoro-2-(propan-2- yloxy)phenyl]-1,3,5-triazin-2- yl}amino)phenyl]methanesulfonamide |
| U5 | 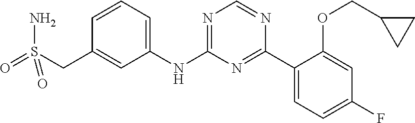 [SMILES: NS(=O)(=O)Cc1cccc(Nc2ncnc(-c3ccc(F)cc3OCC3CC3)n2)c1] | 1-[3-({4-[2-(Cyclopropylmethoxy)-4- fluorophenyl]-1,3,5-triazin-2- yl}amino)phenyl]methanesulfonamide |
| U7 | 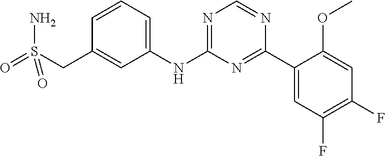 [SMILES: COc1cc(F)c(F)cc1-c1ncnc(Nc2cccc(CS(N)(=O)=O)c2)n1] | 1-(3-{[4-(4,5-Difluoro-2- methoxyphenyl)-1,3,5-triazin-2- yl]amino}phenyl)methanesulfonamide |
| 24 | 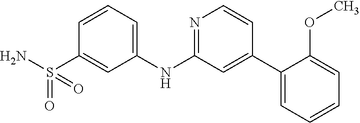 [SMILES: COc1ccccc1-c1ccnc(Nc2cccc(S(N)(=O)=O)c2)c1] | 3-[(4-(2-Methoxyphenyl)pyridin-2- yl)amino]benzenesulfonamide |
| 25 | 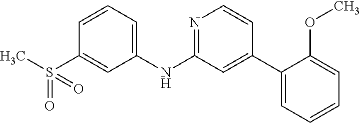 | 4-(2-Methoxyphenyl)-N-(3- (methylsulfonyl)phenyl)pyridin-2-amine |
| 26 | 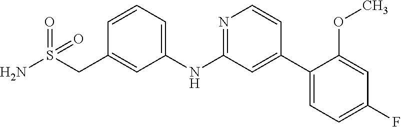 [SMILES: COc1cc(F)ccc1-c1ccnc(Nc2cccc(CS(N)(=O)=O)c2)c1] | [3-((4-(4-Fluoro-2- methoxyphenyl)pyridin-2- yl)amino)phenyl]methanesulfonamide |
| 27 | 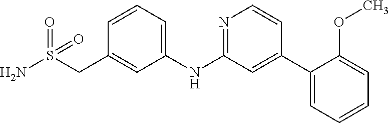 [SMILES: COc1ccccc1-c1ccnc(Nc2cccc(CS(N)(=O)=O)c2)c1] | [3-((4-(2-Methoxyphenyl)pyridin-2- yl)amino)phenyl]methanesulfonamide |
| 28 | 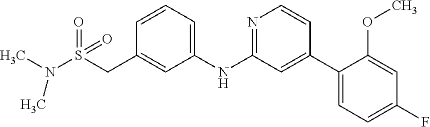 [SMILES: COc1cc(F)ccc1-c1ccnc(Nc2cccc(CS(=O)(=O)N(C)C)c2)c1] | 1-[3-((4-(4-Fluoro-2- methoxyphenyl)pyridin-2- yl)amino)phenyl]-N,N- dimethylmethanesulfonamide |
| 29 | 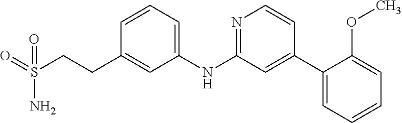 [SMILES: COc1ccccc1-c1ccnc(Nc2cccc(CCS(N)(=O)=O)c2)c1] | 2-[3-((4-(2-Methoxyphenyl)pyridin-2- yl)amino)phenyl]ethanesulfonamide |
| 30 | 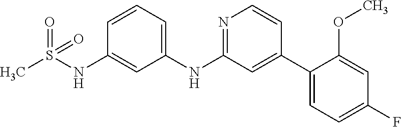 [SMILES: COc1cc(F)ccc1-c1ccnc(Nc2cccc(NS(C)(=O)=O)c2)c1] | N-[3-((4-(4-Fluoro-2- methoxyphenyl)pyridin-2- yl)amino)phenyl]methanesulfonamide |
| 31 | 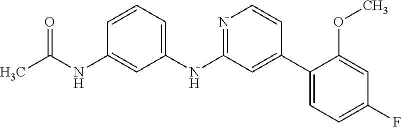 [SMILES: COc1cc(F)ccc1-c1ccnc(Nc2cccc(NC(C)=O)c2)c1] | N-[3-((4-(4-Fluoro-2- methoxyphenyl)pyridin-2- yl)amino)phenyl]acetamide |
| 32 | 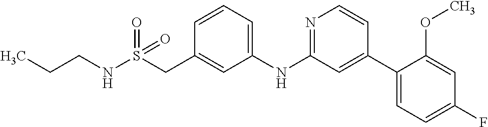 [SMILES: CCCNS(=O)(=O)Cc1cccc(Nc2cc(-c3ccc(F)cc3OC)ccn2)c1] | 1-[3-((4-(4-Fluoro-2- methoxyphenyl)pyridin-2- yl)amino)phenyl]-N- propylmethanesulfonamide |
| 33 | 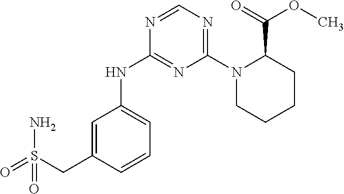 [SMILES: COC(=O)[C@H]1CCCCN1c1ncnc(Nc2cccc(CS(N)(=O)=O)c2)n1] | (R)-Methyl 1-[4-((3- (Sulfamoylmethyl)phenyl)amino)-1,3,5- triazin-2-yl]pipendine-2-carboxylate |
| 34 | 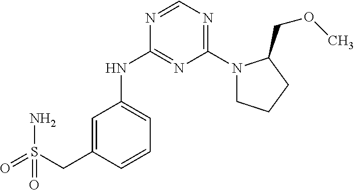 [SMILES: COC[C@H]1CCCN1c1ncnc(Nc2cccc(CS(N)(=O)=O)c2)n1] | (R)-3-[(4-(2-(Methoxymethyl)pyrrolidin- 1-yl)-1,3,5-triazin-2- yl)amino]benzenemethanesulfonamide |
| 35 | 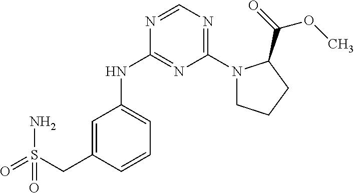 [SMILES: COC(=O)[C@H]1CCCN1c1ncnc(Nc2cccc(CS(N)(=O)=O)c2)n1] | (R)-Methyl 1-[4-((3- (Sulfamoylmethyl)phenyl)amino)-1,3,5- triazin-2-yl]pyrrolidine-2-carboxylate |
| 36 | 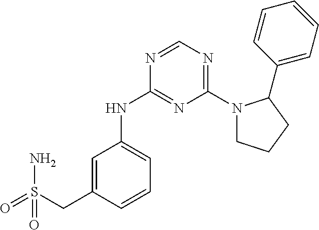 [SMILES: NS(=O)(=O)Cc1cccc(Nc2ncnc(N3CCCC3c3ccccc3)n2)c1] | rac-3-[(4-(2-Phenylpyrrolidin-1-yl)- 1,3,5-triazin-2- yl)amino]benzenemethanesulfonamide |
| 37 | 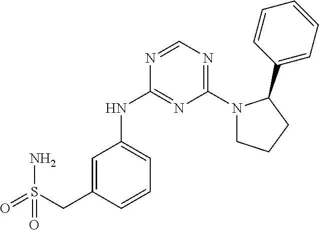 [SMILES: NS(=O)(=O)Cc1cccc(Nc2ncnc(N3CCC[C@@H]3c3ccccc3)n2)c1] | (R)-3-[(4-(2-Phenylpyrrolidin-1-yl)- 1,3,5-triazin-2- yl)amino]benzenemethanesulfonamide |
| 38 | 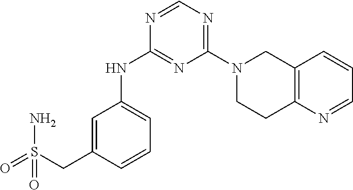 [SMILES: NS(=O)(=O)Cc1cccc(Nc2ncnc(N3CCc4ncccc4C3)n2)c1] | 3-[(4-(7,8-Dihydro-1,6-naphthyridin- 6(5H)-yl)-1,3,5-triazin-2 yl)amino]benzenemethanesulfonamide |
| 39 | 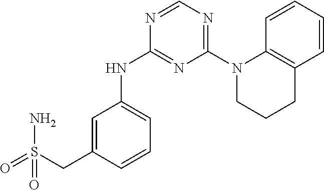 [SMILES: NS(=O)(=O)Cc1cccc(Nc2ncnc(N3CCCc4ccccc43)n2)c1] | 3-[(4-(3,4-Dihydroquinolin-1(2H)-yl)- 1,3,5-triazin-2- yl)amino]benzenemethanesulfonamide |
| 40 | 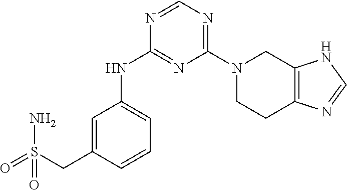 [SMILES: NS(=O)(=O)Cc1cccc(Nc2ncnc(N3CCc4nc[nH]c4C3)n2)c1] | 3-[(4-(6,7-Dihydro-3H-imidazo[4,5- c]pyridin-5(4H)-yl)-1,3,5-triazin-2- yl)amino]benzenemethanesulfonamide |
| 41 | 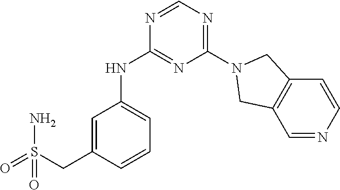 [SMILES: NS(=O)(=O)Cc1cccc(Nc2ncnc(N3Cc4ccncc4C3)n2)c1] | 3-[(4-(1H-Pyrrolo[3,4-c]pyridin-2(3H)- yl)-1,3,5-triazin-2- yl)amino]benzenemethanesulfonamide |
| 42 | 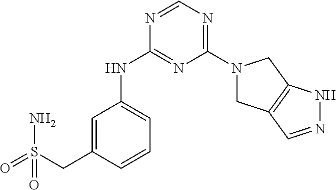 [SMILES: NS(=O)(=O)Cc1cccc(Nc2ncnc(N3Cc4cn[nH]c4C3)n2)c1] | 3-[(4-(Pyrrolo[3,4-c]pyrazol- 5(1H,4H,6H)-yl)-1,3,5-triazin-2- yl)amino]benzenemethanesulfonamide |
| 43 | 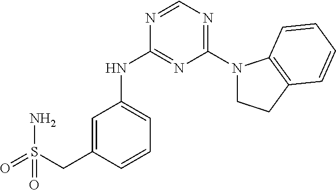 [SMILES: NS(=O)(=O)Cc1cccc(Nc2ncnc(N3CCc4ccccc43)n2)c1] | 3-[(4-(Indolin-1-yl)-1,3,5-triazin-2- yl)amino]benzenemethanesulfonamide |
| 44 | 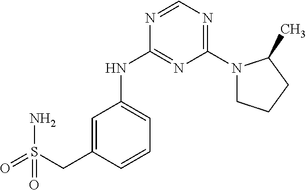 [SMILES: C[C@H]1CCCN1c1ncnc(Nc2cccc(CS(N)(=O)=O)c2)n1] | (S)-3-[(4-(2-Methylpyrrolidin-1-yl)- 1,3,5-triazin-2- yl)amino]benzenemethanesulfonamide |

The above and further compounds which can be used in accordance with the present invention are also disclosed in PCT/EP2011001445, EP10075131.2 (filing date 22 Mar. 2010) EP11075037.9 (filing date 2 Mar. 2011) and EP11075038.7 (filing date 2 Mar. 2011) which are incorporated herein by reference in their entirety.

The compounds of the present invention may form salts with organic or inorganic acids or bases. Examples of suitable acids for such acid addition salt formation are hydrochloric acid, hydrobromic acid, sulfuric acid, phosphoric acid, acetic acid, citric acid, oxalic acid, malonic acid, salicylic acid, p-aminosalicylic acid, malic acid, fumaric acid, succinic acid, ascorbic acid, maleic acid, sulfonic acid, phosphonic acid, perchloric acid, nitric acid, formic acid, propionic acid, gluconic acid, lactic acid, tartaric acid, hydroxymaleic acid, pyruvic acid, phenylacetic acid, benzoic acid, p-aminobenzoic acid, p-hydroxybenzoic acid, methanesulfonic acid, ethanesulfonic acid, nitrous acid, hydroxyethanesulfonic acid, ethylenesulfonic acid, p-toluenesulfonic acid, naphthylsulfonic acid, sulfanilic acid, camphorsulfonic acid, china acid, mandelic acid, o-methylmandelic acid, hydrogen-benzenesulfonic acid, picric acid, adipic acid, d-o-tolyltartaric acid, tartronic acid, (o, m, p)-toluic acid, naphthylamine sulfonic acid, trifluoroacetic acid, and other mineral or carboxylic acids well known to those skilled in the art. The salts are prepared by contacting the free base form with a sufficient amount of the desired acid to produce a salt in the conventional manner. Preferred is the mesylate salt, hydrochloride alt and the trifluoroacetate salt and especially preferred is the trifluoroacetate salt and the hydrochloride salt.

In the case the inventive compounds bear acidic groups, salts could also be formed with inorganic or organic bases. Examples for suitable inorganic or organic bases are, for example, NaOH, KOH, NH₄OH, tetraalkylammonium hydroxide, lysine or arginine and the like. Salts may be prepared in a conventional manner using methods well known in the art, for example by treatment of a solution of the compound of the general formula (I) with a solution of an acid, selected out of the group mentioned above.

### Syntheses of Compounds

The synthesis of the inventive disubstituted triazines according to the present invention is preferably carried out according to the general synthetic sequences, shown in Schemes 1 to 3.

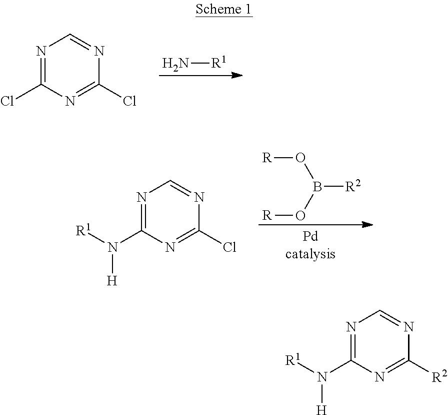

In a first step 2,4-Dichloro-1,3,5-triazine is reacted with anilines R¹NH₂ to give 2-arylamino-4-chloro-1,3,5-triazines. The reaction is carried out with one equivalent of the aniline in an inert solvent like DMF, THF, DME, dioxane or an alcohol like isopropanol, or mixtures of such solvents. Preferably the reaction is carried out at a temperature below room temperature in such a way that the reaction mixture is kept homogenous. Preferred conditions use an additional base like triethylamine or N,N-diisopropylethylamine.

In a second step the intermediate 2-arylamino-4-chloro-1,3,5-triazine is reacted with a boronic acid derivative R²—B(OR)₂ to give compounds of Formula (I). The boronic acid derivative may be a boronic acid (R═H) or an ester of the boronic acid, e.g. its isopropyl ester (R═—CH(CH₃)₂), preferably an ester derived from pinacol in which the boronic acid intermediate forms a 2-aryl-4,4,5,5-tetramethyl-1,3,2-dioxaborolane (R—R═—C(CH₃)₂—C(CH₃)₂—. Both R represent independently of each other preferably hydrogen or an alkyl chain with 1-10 carbon atoms or a cycloalkyl chain with 3 to 12 carbon atoms or both residues R represent together a residue derived from pinacol. The coupling reaction is catalyzed by Pd catalysts, e.g. by Pd(0) catalysts like tetrakis(triphenylphosphine)palladium(0) [Pd(PPh₃)₄], tris(dibenzylideneacetone)di-palladium(0) [Pd₂(dba)₃], or by Pd(II) catalysts like dichlorobis(triphenylphosphine)-palladium(II) [Pd(PPh₃)₂Cl₂], palladium(II) acetate and triphenylphosphine or more preferred by [1,1′-bis(diphenylphosphino)ferrocene]palladium dichloride. The reaction is preferably carried out in a mixture of a solvent like dioxane, DMF, DME, THF, or isopropanol with water and in the presence of a base like aqueous sodium bicarbonate or K₃PO₄.

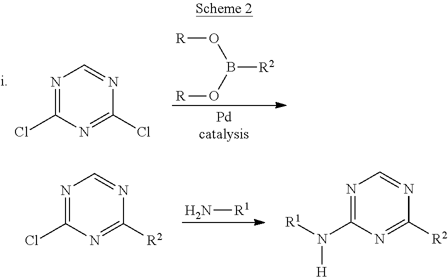

The synthesis of 1,3,5-triazines of Formula (I) starting from 2,4-dichloro-1,3,5-triazine may be carried out in the inverse order of the reaction steps compared to Scheme 1, in such a manner that in a first step the reaction of a triazine with a boronic acid derivative is followed in a second step by the reaction of the intermediate triazine with an aniline. Preferred conditions for the coupling reaction of the first step are heating the reacting agents in toluene with dichlorobis(triphenylphosphine)palladium(II) [Pd(PPh₃)₂Cl₂] as a catalyst in the presence of sodium or potassium carbonate as a base.

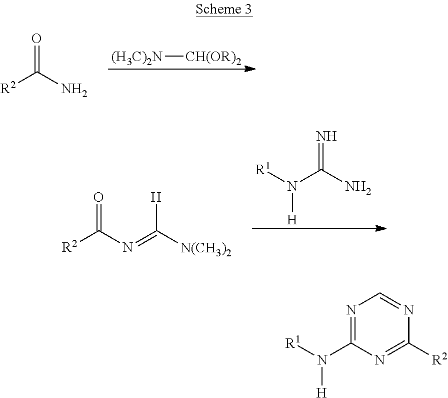

Compounds of Formula (I) may be prepared by the methodology described in J. Org. Chem. 60 (1995), 8428-8430. Primary amides R²—CONH₂ are heated with acetals and preferably dialkylacetals of N,N-dimethylformamide, preferably with its dimethyl or diethyl acetal, in particular with the dimethyl acetal (R═—CH₃). The intermediate N-acylformamidine is not isolated and subsequently converted to 1,3,5-triazines of Formula (I) by heating with a guanidine R¹—NH—C(NH)NH₂. Preferably the reaction is carried out by heating the reacting agents in dioxane in the presence of a base like potassium tert-butoxide.

Several compounds of Formula (I) may be prepared by converting substituents which are attached to the aromatic rings R¹ and/or R² to other substituents using standard reactions which are known to the person skilled in the art. For example, a nitro group can be reduced to an amino group, such an amino group can be converted to a sulfonamide by reaction with a sulfonyl chloride, to a carboxamide by reaction with a carbonyl chloride or another activated derivative of a carboxylic acid, to an urea by reaction with an isocyanate. Carbamate substituents may be cleaved to amino groups, in particular tert-butyl carbamates by reaction with acids like trifluoroacetic acid or hydrochloric acid. Formyl groups may be converted to aminomethyl groups by reaction with primary amines under conditions of a reductive amination; see, for example, synthesis of the compounds as shown in Table 2.

Further CDK9 inhibitors to be used in accordance with the present invention are well known in the art and are, for example, described in Krystof (2009) Medicinal Research Reviews, DOI 10.1002/med.20172, as well as in international patent applications published as WO 2009047359, WO 2010003133, WO 200879933 and WO 2011012661. All these documents are incorporated herein by reference in their entirety.

Potential CDK9 inhibitors, especially selective CDK9 inhibitors, as defined herein above may be screened/identified by routine assays, such as a radiometric protein kinase assay (33 PanQinase® Activity Assay; and/or the well known Lance Assay.

The following exemplary inhibitors can be used in accordance with the present invention, for example, in cotherapy as described herein: SNS-032: Piperidine-4-carboxylic acid [5-(5-tert-butyl-oxazol-2-ylmethylsulfanyl)-thiazol-2-yl]-amide; Misra R N et al. J Med. Chem. 2004, 47(7):1719-28;

flavopiridol: 2-(2-Chloro-phenyl)-5,7-dihydroxy-8-(3-hydroxy-1-methyl-piperidin-4-yl)-chromen-4-one, Lloyd R Kelland. Expert Opinion on Investigational Drugs. 2000, 9(12):2903-2911;

AX35427: N-(5-((6-(3-aminophenyl)pyrimidin-4-yl)amino)-2-methylphenyl)propane-1-sulfonamide, 848637-29-6P in WO 2005026129;

R-547: [4-amino-2-(1-methanesulfonylpiperidin-4-ylamino)pyrimidin-5-yl]-(2,3-difluoro-6-methoxyphenyl)methanone, DePinto W et al., Mol Cancer Ther 2006, 5:2644-2658;

1073485-20-7P: 3-[[6-(2-methoxyphenyl)-4-pyrimidinyl]amino]-Benzenemethanesulfonamide compound 1073485-20-7P in WO 2008132138.

AX38679: 3-((6-(2-methoxyphenyl)pyrimidin-4-yl)amino)benzenesulfonamide, 848637-62-7P in WO 2005026129;

PHA767491: 1,5,6,7-tetrahydro-2-(4-pyridinyl)-4H-pyrrolo[3,2-c]pyridin-4-one, Montagnoli, A. Nat Chem Biol 2008, 4(6) 357-365;

BS181: N5-(6-aminohexyl)-3-(1-methylethyl)-N-7-(phenylmethyl), Ali S et al. Cancer Res. 2009, 69(15):6208-15);

DRB:

5,6-dichloro-1-b-ribofuranosyl-benzimidazole; Mancebo H S, Lee G, Flygare J, Tomassini J, Luu P, Zhu Y, Peng J, Blau C, Hazuda D, Price D, Flores O. P-TEFb kinase is required for HIV Tat transcriptional activation in vivo and in vitro. Genes Dev 1997; 11:2633-2644;

Roscovitine: 6-Benzylamino-2[(R)-(1′-ethyl-2′-hydroxyethylamino)]-9-isopropylpurine, Meijer, Laurent; Bisagni, Emile; Legraverend, Michel. Purine derivatives with antiproliferative properties, their preparation, and biological uses thereof. PCT Int. Appl. (1997), 52 pp. WO 9720842 A1;

AG-012986:

4-[[4-Amino-5-(2,6-difluorobenzoyl)thiazol-2-yl]amino]-N-((R)-2-dimethylamino-1-methylethyl)benzamide Zhang C, Lundgren K, Yan Z, Arango M E, Price S, Huber A, Higgins J, Troche G, Skaptason J, Koudriakova T, Nonomiya J, Yang M, O'Connor P, Bender S, Los G, Lewis C, Jessen B. Pharmacologic properties of AG-012986, a pan-cyclin-dependent kinase inhibitor with antitumor efficacy. Mol Cancer Ther 2008; 7:818-828;

P276-00:

4H-1-Benzopyran-4-one, 2-(2-chlorophenyl)-5,7-dihydroxy-8-[(2R,3S)-2-(hydroxymethyl)-1-methyl-3-pyrrolidinyl]-, hydrochloride (1:1); Joshi, Kalpana S.; Rathos, Maggie J.; Joshi, Rajendra D.; Sivakumar, Meenakshi; Mascarenhas, Malcolm; Kamble, Shrikant; Lal, Bansi; Sharma, Somesh. In vitro antitumor properties of a novel cyclin-dependent kinase inhibitor, P276-00. Molecular Cancer Therapeutics (2007), 6(3), 918-925. CODEN: MCTOCF ISSN:1535-7163. CAN 146:513967 AN 2007:289479;

ZK 304709:

4-[3-Chloro-5-(4-methylpiperazin-1-yl)benzoylamino]-1H-pyrazole-3-carboxylic acid cyclohexylamide Siemeister, G.; Luecking, U.; Wagner, C.; Detjen, K.; Mc Coy, C.; Bosslet, Klaus. Molecular and pharmacodynamic characteristics of the novel multi-target tumor growth inhibitor ZK304709. Biomedicine & Pharmacotherapy (2006), 60(6), 269-272. CODEN: BIPHEX ISSN:0753-3322. CAN 146:176348 AN 2006:831518;

EXEL-8647 and/or EXEL-3700:

Heuer T S. Discovery of Selective CDK9 Small Molecule Inhibitors: CDK9 Inhibition in Tumor Cells is Associated with Inhibition of Proliferation and Induction of Apoptosis. AACR-NCIEORTC International Conference on Molecular Targets and Cancer Therapeutics. Geneva, Switzerland; 21-24 Oct. 2008;

AT7519:

4-(2,6-Dichlorobenzoylamino)-1H-pyrazole-3-carboxylic acid N-(piperidin-4-yl)amide Squires M S, Feltell R E, Lock V, Smith D, Lewis E J, Higgins J, Yule M, Thompson N T, Cooke L, Croce Della K, Qi W, Lyons J F, Mahadevan D. AT7519, a potent CDK inhibitor, is active in leukemia models and primary CLL patient samples. 49th Annual Meeting and Exposition of American Society for Hematology. Atlanta, Ga., 8-11 Dec. 2007;

Compound 7d:

N-[2-(dimethylamino)ethyl]-2-fluoro-4-[[5-fluoro-4-[2-methyl-1-(1-methylethyl)-1H-imidazol-5-yl]-2-pyrimidinyl]amino]; Jones C D, Andrews D M, Barker A J, Blades K, Daunt P, East S, Geh C, Graham M A, Johnson K M, Loddick S A, McFarland H M, McGregor A, Moss L, Rudge D A, Simpson P B, Swain M L, Tam K Y, Tucker J A, Walker M. The discovery of AZD5597, a potent imidazole pyrimidine amide CDK inhibitor suitable for intravenous dosing. Bioorg Med Chem Lett 2008; 18:6369-6373;

AZD5597:

[4-[[5-fluoro-4-[2-methyl-1-(1-methylethyl)-1H-imidazol-5-yl]-2-pyrimidinyl]amino]phenyl][(3S)-3-(methylamino)-1-pyrrolidinyl]-Jones C D, Andrews D M, Barker A J, Blades K, Daunt P, East S, Geh C, Graham M A, Johnson K M, Loddick S A, McFarland H M, McGregor A, Moss L, Rudge D A, Simpson P B, Swain M L, Tam K Y, Tucker J A, Walker M. The discovery of AZD5597, a potent imidazole pyrimidine amide CDK inhibitor suitable for intravenous dosing. Bioorg Med Chem Lett 2008; 18:6369-6373;

RGB-286638:

N-[1,4-dihydro-3-[4-[[4-(2-methoxyethyl)-1-piperazinyl]methyl]phenyl]-4-oxoindeno[1,2-c]pyrazol-5-yl]-N′-4-morpholinyl-, hydrochloride (1:2) Pharmacological targeting of CDK9 in cardiac hypertrophy Krystof V, Chamrad I, Jorda R, Kohoutek J. Med Res Rev. 2010 July; 30(4):646-66. Review;

LCQ195AT9311:

4-(2,6-dichlorobenzamido)-N-(1-(methylsulfonyl)piperidin-4-yl)-1H-pyrazole-3-carboxamide McMillin, Douglas W.; Delmore, Jake; Negri, Joseph; Buon, Leutz; Jacobs, Hannah M.; Laubach, Jacob; Jakubikova, Jana; Ooi, Melissa; Hayden, Patrick; Schlossman, Robert; Munshi, Nikhil c.; Lengauer, Christoph; Richardson, Paul G.; Anderson, Kenneth C.; Mitsiades, Constantine S. Molecular and cellular effects of multi-targeted cyclin-dependent kinase inhibition in myeloma: biological and clinical implications. British Journal of Haematology (2011), 152(4), 420-432. CODEN: BJHEAL ISSN:0007-1048. AN 2011:307444 CAPLUS.

Particularly preferred compounds for use in the present invention are Cpd 24, Cpd C1, Cpd B1 and Cpd B2 as described and defined herein above.

Also siRNAs/RNAis, antisense molecules and ribozymes directed against nucleic acid molecules encoding CDK9 or its activators Cyclin T or Cyclin K are envisaged as (an) CDK9 inhibitor(s) for the use and the method of the present invention. The above-mentioned antagonist/inhibitor of CDK9 may also be a co-suppressive nucleic acid.

An siRNA approach is, for example, disclosed in Elbashir ((2001), Nature 411, 494-498)). It is also envisaged in accordance with this invention that for example short hairpin RNAs (shRNAs) are employed in accordance with this invention as pharmaceutical composition. The shRNA approach for gene silencing is well known in the art and may comprise the use of st (small temporal) RNAs; see, inter alia, Paddison (2002) Genes Dev. 16, 948-958.

As mentioned above, approaches for gene silencing are known in the art and comprise “RNA”-approaches like RNAi (iRNA) or siRNA. Successful use of such approaches has been shown in Paddison (2002) loc. cit., Elbashir (2002) Methods 26, 199-213; Novina (2002) Mat. Med. Jun. 3, 2002; Donze (2002) Nucl. Acids Res. 30, e46; Paul (2002) Nat. Biotech 20, 505-508; Lee (2002) Nat. Biotech. 20, 500-505; Miyagashi (2002) Nat. Biotech. 20, 497-500; Yu (2002) PNAS 99, 6047-6052 or Brummelkamp (2002), Science 296, 550-553. These approaches may be vector-based, e.g. the pSUPER vector, or RNA polIII vectors may be employed as illustrated, inter alia, in Yu (2002) loc. cit.; Miyagishi (2002) loc. cit. or Brummelkamp (2002) loc. cit.

However, use of CDK9 inhibitors in accordance with the present invention is not limited to known CDK9 inhibitors. Accordingly, also yet unknown CDK9 inhibitors may be used in accordance with the present invention. Such inhibitors may be identified by the methods described and provided herein and methods known in the art, like high-throughput screening using biochemical assays for inhibition of CDK9. Assays for screening of potential CDK9 inhibitors and, in particular, for identifying selective CDK9 inhibitors as defined herein are shown in the experimental part and described herein above. For example, a radiometric protein kinase assay (33 PanQinase® Activity Assay; see FIG. 3. In addition/in the alternative, the well known Lance Assay can also be used; see FIG. 2. Based on his general knowledge a person skilled in the art is easily in the position to identify inhibitors or verify the inhibiting activity of compounds suspected of being CDK9 inhibitors. These tests may be employed on cell(s) or cell culture(s) described in the appended example, but also further cell(s)/tissue(s)/cell culture(s) may be used, such as cell(s)/tissue(s)/cell culture(s) derived from biopsies.

The following refers to screening or validating potential CDK9 inhibitors, especially selective CDK9 inhibitors to be used in accordance with the present invention. For example, the activity/level of expression of CDK9 may be determined, wherein a lower activity/level of expression of CDK9 compared to a control is indicative for the capacity of a candidate molecule/substance to selectively inhibit CDK9. The term “activity of CDK9” used herein refers to the activity of a CDK9 protein (protein encoded by a CDK9 gene). The term “expression of CDK9” is used herein interchangeably with “expression of CDK9 gene” and refers to the expression of the CDK9 gene. It is to be understood that the activity/expression level of CDK9 determined in (a) cell(s), (a) tissue(s) or (a) cell culture(s) contacted with/exposed to an CDK9 inhibitor is compared with the activity/expression level of CDK9 in (a) control cell(s), (a) tissue(s) or (a) cell culture(s), i.e. cell(s), (a) tissue(s) or (a) cell culture(s) not contacted with/exposed to an CDK9 inhibitor. A skilled person will be aware of means and methods for performing such tests and selecting appropriate controls. Preferably, the control cell(s), (a) tissue(s) or (a) cell culture(s) will be identical to the cell(s), (a) tissue(s) or (a) cell culture(s) to be tested as described herein with the only exception that the control (s), (a) tissue(s) or (a) cell culture(s) are not contacted with/exposed to the CDK9 inhibitor.

Preferably, decreased CDK9 activity/expression levels of CDK9 proteins/polypeptides and/or CDK9 polynucleotides/nucleic acid molecules are indicative of the capacity of a candidate molecule/substance to selectively inhibit CDK9. It is preferred herein that the CDK9 activity/expression level is decreased by at least 10%, 20%, 30%, 40%, 50%, 60%, 70%, 80% and most preferably by at least 90% in cell(s), (a) tissue(s) or (a) cell culture(s) contacted with/exposed to an CDK9 inhibitor compared with the activity/expression level of CDK9 in (a) control cell(s), (a) control tissue(s) or (a) control cell culture(s). It is of note that the CDK9 activity must not necessarily correlate with the expression level. Thus, it may be, that CDK9 activity is decreased in the presence of an CDK9 inhibitor even though CDK9 expression is the same or even increased. However, a person skilled of the art will be aware of this and preferably evaluate CDK9 activity (i.e. activity/function of the CDK9 protein) when determining the capacity of a candidate substance to inhibit CDK9.

As mentioned, a person skilled in the art will be aware of corresponding means and methods for detecting and evaluating the CDK9 activity/expression level. Exemplary methods to be used include but are not limited to molecular assessments such as Western Blots, Northern Blots, Real-Time PCR and the like.

If the gene product is an RNA, in particular an mRNA (e.g. unspliced, partially spliced or spliced mRNA), determination can be performed by taking advantage of northern blotting techniques, hybridization on microarrays or DNA chips equipped with one or more probes or probe sets specific for mRNA transcripts or PCR techniques referred to above, like, for example, quantitative PCR techniques, such as Real time PCR. These and other suitable methods for binding (specific) mRNA are well known in the art and are, for example, described in Sambrook and Russell (2001, loc. cit.). A skilled person is capable of determining the amount of the component, in particular said gene products, by taking advantage of a correlation, preferably a linear correlation, between the intensity of a detection signal and the amount of the gene product to be determined.

In case the component is a polypeptide/protein, quantification can be performed by taking advantage of the techniques referred to above, in particular Western blotting techniques. Generally, the skilled person is aware of methods for the quantitation of (a) polypeptide(s)/protein(s). Amounts of purified polypeptide in solution can be determined by physical methods, e.g. photometry. Methods of quantifying a particular polypeptide in a mixture rely on specific binding, e.g of antibodies. Specific detection and quantitation methods exploiting the specificity of antibodies comprise for example immunohistochemistry (in situ). Western blotting combines separation of a mixture of proteins by electrophoresis and specific detection with antibodies. Electrophoresis may be multi-dimensional such as 2D electrophoresis. Usually, polypeptides are separated in 2D electrophoresis by their apparent molecular weight along one dimension and by their isoelectric point along the other direction. Alternatively, protein quantitation methods may involve but are not limited to mass spectrometry or enzyme-linked immunosorbant assay methods.

Also the use of high throughput screening (HTS) is envisaged in context of the present invention, in particular the screening methods for potential CDK9 inhibitors to be used herein. Suitable (HTS) approaches are known in the art and a person skilled in the art is readily in the position to adapt such protocols or known HTS approaches to the performance of the methods of the present invention.

Screening-assays are usually performed in liquid phase, wherein for each cell/tissue/cell culture to be tested at least one reaction batch is made. Typical containers to be used are micro titer plates having for example, 384, 1536, or 3456 wells (i.e. multiples of the “original” 96 reaction vessels).

Robotics, data processing and control software, and sensitive detectors, are further commonly used components of a HTS device. Often robot system are used to transport micro titer plates from station to station for addition and mixing of sample(s) and reagent(s), incubating the reagents and final readout (detection). Usually, HTS can be used in the simultaneous preparation, incubation and analysis of many plates.

The assay can be performed in a singly reaction (which is usually preferred), may, however, also comprise washing and/or transfer steps. Detection can be performed taking advantage of radioactivity, luminescence or fluorescence, like fluorescence-resonance-energytransfer (FRET) and fluorescence polarisation (FP) and the like. The biological samples described herein can also be used in such a context. In particular cellular assays and in vivo assays can be employed in HTS. Cellular assays may also comprise cellular extracts, i.e. extracts from cells, tissues and the like. However, preferred herein is the use of cell(s) or tissue(s) as biological sample (in particular a sample obtained from a patient/subject suffering or being prone to suffer from midline carcinoma, especially NMC), whereas in vivo assays (wherein suitable animal models are employed, e.g. the herein described mouse models) are particularly useful in the validation of potential CDK9 inhibitors. Depending on the results of a first assay, follow up assays can be performed by re-running the experiment to collect further data on a narrowed set (e.g. samples found “positive” in the first assay), confirming and refining observations.

HTS is useful in identifying further CDK9 inhibitors to be used herein. The screening of compound libraries with usually several hundred thousands of substances takes usually between days and weeks. An experimental high throughput screen may be supplemented (or even be replaced) by a virtual screen. For example, if the structure of the target molecule (e.g. CDK9) is known, methods can be employed, which are known under the term “docking”. If the structure of several target-binding molecules is known (e.g. the herein described CDK9) methods for Pharmacophor-Modelling can be used aiming at the development new substances which also bind to the target molecule. A suitable readout in animal (in vivo) models is tumor growth (or respectively the complete or partial inhibition of tumor growth and/or its remission).

High-throughput methods for the detection of mutations involve massively parallel sequencing approaches, such as the “picotiter plate pyrosequencing”. This approach relies on emulsion PCR-based clonal amplification of a DNA library adapted onto micron-sized beads and subsequent pyrosequencing-by-synthesis (Thomas R K et al. Nature Med 2007) of each clonally amplified template in a picotiter plate, generating over 200,000 unique clonal sequencing reads per experiment. Furthermore, mass spectrometric genotyping approaches (Thomas R K et al.; Nat Gen 2007) and other next generation sequencing methods (Marguerat S et al.; Biochem Soc Trans 2008) may be employed.

The meaning of the terms “cell(s)”, “tissue(s)” and “cell culture(s)” is well known in the art and may, for example, be deduced from “The Cell” (Garland Publishing, Inc., third edition). Generally, the term “cell(s) used herein refers to a single cell or a plurality of cells. The term “plurality of cells” means in the context of the present invention a group of cells comprising more than a single cell. Thereby, the cells out of said group of cells may have a similar function. Said cells may be connected cells and/or separate cells. The term “tissue” in the context of the present invention particularly means a group of cells that perform a similar function. The term “cell culture(s)” means in context of the present invention cells as defined herein above which are grown/cultured under controlled conditions. Cell culture(s) comprise in particular cells (derived/obtained) from multicellular eukaryotes, preferably animals as defined elsewhere herein. It is to be understood that the term “cell culture(s)” as used herein refers also “tissue culture (s)” and/or “organ culture(s)”, an “organ” being a group of tissues which perform the some function.

Preferably, the cell(s), tissue(s) or cell culture(s) to be contacted with/exposed to a selective CDK9 inhibitor comprise/are derived from or are (a) tumor cell(s). The tumor cells may, for example, be obtained from a biopsy, in particular a biopsy/biopsies from a patient/subject suffering from midline carcinoma, like NMC or, though less preferred a patient/subject being prone to suffer from midline carcinoma, like NMC. It is preferred herein that said subject is a human. The term “mammalian tumor cell(s)” used herein refers to (a) tumor cell(s) which is derived from or is a tumor cell from a mammal, the term mammal being derived herein below. As described herein above in respect of “cell(s)”, “tissue(s)” and “cell culture(s)” the “mammalian tumor cells” may be obtained from a biopsy, in particular a biopsy/biopsies from a patient/subject suffering from midline carcinoma, like NMC or, though less preferred a patient/subject being prone to suffer from midline carcinoma, like NMC. The term “tumor cell” also relates to “cancer cells”.

Generally, said tumor cell or cancer cell may be obtained from any biological source/organism, particularly any biological source/organism, suffering from the above-mentioned midline carcinoma, like NMC.

Preferably, the (tumor) cell(s) or (cancer) cell to be contacted is (are) obtained/derived from an animal. More preferably, said (tumor)(cancer) cell(s) is (are) derived from a mammal. The meaning of the terms “animal” or “mammal” is well known in the art and can, for example, be deduced from Wehner und Gehring (1995; Thieme Verlag). Non-limiting examples for mammals are even-toed ungulates such as sheep, cattle and pig, odd-toed angulates such as horses as well as carnivores such as cats and dogs. In the context of this invention, it is particularly envisaged that DNA samples are derived from organisms that are economically, agronomically or scientifically important. Scientifically or experimentally important organisms include, but are not limited to, mice, rats, rabbits, guinea pigs and pigs.

The tumor cell(s) may also be obtained from primates which comprise lemurs, monkeys and apes. The meaning of the terms “primate”, “lemur”, “monkey” and “ape” is known and may, for example, be deduced by an artisan from Wehner und Gehring (1995, Thieme Verlag). As mentioned above, the tumor or cancer cell(s) is (are) most preferably derived from a human being suffering from the above-mentioned NMCs. In context of this invention particular useful cells, in particular tumor or cancer cells, are, accordingly, human cells. These cells can be obtained from e.g. biopsies or from biological samples but the term “cell” also relates to in vitro cultured cells.

A preferred, however non-limiting cell(s) or cell culture(s) also used in the appended example is cell line 143100 (showing a t15;19 translocation resulting in the formation of a BRD4-NUT-fusion protein). A further cell line to be used in accordance with the present invention is HCC2429 (showing NOTCH3 overexpression in addition to the t15;19 translocation). Further cell lines that can be used include HCC1143 (NOTCH3 overexpression), PC9 (EGFRmut) or A549 (KRAS mut). These cell lines are well known in the art and may be obtained from ATCC and/or DSMZ and/or from the U.S. National Cancer Institute (www.lgcpromochem-atcc.com/; www.dsmz.de/; dtp.nci.nih.gov/docs/misc/common_files/cell_list.html).

The following explanations refer, inter alia, to rearrangements in the NUT gene which is characteristic especially for NUT midline carcinoma.

The below explanations concerning the NUT gene and NUT protein, apply, mutatis mutandis, to other nucleic acid sequences and amino acid sequences to be employed in context of the present invention, such as partner genes of NUT in NUT fusion genes like BRD4-NUT fusion genes, BRD3-NUT fusion genes or NUT-variant fusion genes characteristic for NMC. Accordingly, the explanations apply, mutatis mutandis, to members of the BET family (BRD2, BRDT and, in particular, human BRD3 gene and BRD3 protein (SEQ ID NOs: 5 and 6, respectively) and human BRD4 gene and BRD4 protein (SEQ ID NOs: 3 and 4, respectively). The explanations apply also to human CDK9 gene and CDK9 protein (SEQ ID NOs: 7 and 8, respectively), in particular CDK9 proteins to be used in the screening and/or validation of potential selective CDK9 inhibitors as described herein.

The term “NUT gene” (“nuclear protein in testis”) as used in context of this invention refers to a gene encoding an unstructured polypeptide of unknown function that is highly expressed in normal spermatids; see Schwartz, loc. cit. It has been reported that the NUT protein binds to the histone acetyltransferase (HAT) p300; see Schwartz, loc. cit.

The nucleic acid sequence of the human NUT gene and the corresponding amino acid sequence is shown in SEQ ID NO: 1 and SEQ ID NO: 2, respectively. Generally, the term NUT used herein refers to any amino acid sequence having (partial) NUT activity as described herein and nucleic acid sequence(s) encoding such (an) amino acid sequence(s).

The nucleic acid sequences of NUT of other mammalian or non-mammalian species (in particular mouse, rat, chimpanzee) than the herein provided sequences for human NUT can be identified by the skilled person using methods known in the art, e.g. by nucleic acid sequencing or using hybridization assays or by using alignments, either manually or by using computer programs such as those mentioned herein below in connection with the definition of the term “hybridization” and degrees of homology. In one embodiment, the nucleic acid sequence encoding for orthologs of human NUT gene is at least 40% homologous to the nucleic acid sequences as shown in SEQ ID NO: 1. More preferably, the nucleic acid sequence encoding for orthologs of the human NUT gene is at least 45%, 50%, 55%, 60%, 65%, 70%, 75%, 80%, 85%, 90%, 91%, 92%, 93%, 94%, 95%, 96%, 97% or 98% homologous to the nucleic acid sequence as shown in SEQ ID NO. 1, wherein the higher values are preferred. Most preferably, the nucleic acid sequence encoding for orthologs of the human NUT gene is at least 99% homologous to the nucleic acid sequence as shown in SEQ ID NO. 1.

Hybridization assays for the characterization of orthologs of known nucleic acid sequences/promoters are well known in the art; see e.g. Sambrook, Russell “Molecular Cloning, A Laboratory Manual”, Cold Spring Harbor Laboratory, N.Y. (2001); Ausubel, “Current Protocols in Molecular Biology”, Green Publishing Associates and Wiley Interscience, N.Y. (1989). The term “hybridization” or “hybridizes” as used herein may relate to hybridizations under stringent or non-stringent conditions. If not further specified, the conditions are preferably non-stringent. Said hybridization conditions may be established according to conventional protocols described, e.g., in Sambrook (2001) loc. cit.; Ausubel (1989) loc. cit., or Higgins and Hames (Eds.) “Nucleic acid hybridization, a practical approach” IRL Press Oxford, Washington D.C., (1985). The setting of conditions is well within the skill of the artisan and can be determined according to protocols described in the art. Thus, the detection of only specifically hybridizing sequences will usually require stringent hybridization and washing conditions such as, for example, the highly stringent hybridization conditions of 0.1×SSC, 0.1% SDS at 65° C. or 2×SSC, 60° C., 0.1% SDS. Low stringent hybridization conditions for the detection of homologous or not exactly complementary sequences may, for example, be set at 6×SSC, 1% SDS at 65° C. As is well known, the length of the probe and the composition of the nucleic acid to be determined constitute further parameters of the hybridization conditions.

In accordance with the present invention, the terms “homology” or “percent homology” or “identical” or “percent identity” or “percentage identity” or “sequence identity” in the context of two or more nucleic acid sequences refers to two or more sequences or subsequences that are the same, or that have a specified percentage of nucleotides that are the same (preferably at least 40% identity, more preferably at least 45%, 50%, 55%, 60%, 65%, 70%, 75%, 80%, 85%, 90%, 91%, 92%, 93%, 94%, 95%, 96%, 97% or 98% identity, most preferably at least 99% identity), when compared and aligned for maximum correspondence over a window of comparison, or over a designated region as measured using a sequence comparison algorithm as known in the art, or by manual alignment and visual inspection. Sequences having, for example, 75% to 90% or greater sequence identity may be considered to be substantially identical. Such a definition also applies to the complement of a test sequence. Preferably the described identity exists over a region that is at least about 15 to 25 nucleotides in length, more preferably, over a region that is at least about 50 to 100 nucleotides in length, more preferably at least about 200 to 400 nucleotides, at least about 300 to 500 nucleotides, at least about 400 to 600 nucleotides in length, at least about 500 to 1000 nucleotides, at least about 800 to 1500 nucleotides, at least about 1000 to 2000 nucleotides, at least about 1500 to 2500 nucleotides or at least about 2000 to 3000 nucleotides. Even more preferably, the described identity exists over a region that is at least about 3000 to 4200 nucleotides in length, more preferably at least about 3200 to 4000 nucleotides, more preferably at least about 3300 to 3900 nucleotides. Most preferably, the described identity exists over a region that is at least about 3350 to 3850 nucleotides in length. In a most preferred embodiment, the described identity exists over the entire length of the nucleotide sequence shown in SEQ ID NO. 1, preferably the region thereof encoding the NUT protein. The coding region ranges from nucleotide 156 to nucleotide 3554 of the nucleotide sequence shown in SEQ ID NO: 1. Those having skill in the art will know how to determine percent identity between/among sequences using, for example, algorithms such as those based on CLUSTALW computer program (Thompson Nucl. Acids Res. 2 (1994), 4673-4680) or FASTDB (Brutlag Comp. App. Biosci. 6 (1990), 237-245), as known in the art.

Although the FASTDB algorithm typically does not consider internal non-matching deletions or additions in sequences, i.e., gaps, in its calculation, this can be corrected manually to avoid an overestimation of the % identity. CLUSTALW, however, does take sequence gaps into account in its identity calculations. Also available to those having skill in this art are the BLAST and BLAST 2.0 algorithms (Altschul, (1997) Nucl. Acids Res. 25:3389-3402; Altschul (1993) J. Mol. Evol. 36:290-300; Altschul (1990) J. Mol. Biol. 215:403-410). The BLASTN program for nucleic acid sequences uses as defaults a word length (W) of 11, an expectation (E) of 10, M=5, N=4, and a comparison of both strands. The BLOSUM62 scoring matrix (Henikoff (1989) PNAS 89:10915) uses alignments (B) of 50, expectation (E) of 10, M=5, N=4, and a comparison of both strands.

In order to determine whether a nucleotide residue in a nucleic acid sequence corresponds to a certain position in the nucleotide sequence of e.g. SEQ ID NO 1, the skilled person can use means and methods well-known in the art, e.g., alignments, either manually or by using computer programs such as those mentioned herein. For example, BLAST 2.0, which stands for Basic Local Alignment Search Tool BLAST (Altschul (1997), loc. cit.; Altschul (1993), loc. cit.; Altschul (1990), loc. cit.), can be used to search for local sequence alignments. BLAST, as discussed above, produces alignments of nucleotide sequences to determine sequence similarity. Because of the local nature of the alignments, BLAST is especially useful in determining exact matches or in identifying similar sequences. The fundamental unit of BLAST algorithm output is the High-scoring Segment Pair (HSP). An HSP consists of two sequence fragments of arbitrary but equal lengths whose alignment is locally maximal and for which the alignment score meets or exceeds a threshold or cut-off score set by the user. The BLAST approach is to look for HSPs between a query sequence and a database sequence, to evaluate the statistical significance of any matches found, and to report only those matches, which satisfy the user-selected threshold of significance. The parameter E establishes the statistically significant threshold for reporting database sequence matches. E is interpreted as the upper bound of the expected frequency of chance occurrence of an HSP (or set of HSPs) within the context of the entire database search. Any database sequence whose match satisfies E is reported in the program output.

Analogous computer techniques using BLAST (Altschul (1997), loc. cit.; Altschul (1993), loc. cit.; Altschul (1990), loc. cit.) are used to search for identical or related molecules in nucleotide databases such as GenBank or EMBL. This analysis is much faster than multiple membrane-based hybridizations. In addition, the sensitivity of the computer search can be modified to determine whether any particular match is categorized as exact or similar. The basis of the search is the product score which is defined as:

$$\frac{\% sequence identity \times \% maximum B L A S T score}{100}$$

and it takes into account both the degree of similarity between two sequences and the length of the sequence match. For example, with a product score of 40, the match will be exact within a 1-2% error; and at 70, the match will be exact. Similar molecules are usually identified by selecting those which show product scores between 15 and 40, although lower scores may identify related molecules. Another example for a program capable of generating sequence alignments is the CLUSTALW computer program (Thompson (1994) Nucl. Acids Res. 2:4673-4680) or FASTDB (Brutlag (1990) Comp. App. Biosci. 6:237-245), as known in the art.

Also envisaged herein is not only the use of nucleic acid sequences encoding the NUT gene but also amino acid sequences of NUT protein. In line with the above explanations concerning orthologs/homologs of human NUT gene as shown in SEQ ID NO: 2, also orthologous/homologous amino acid sequences of the human NUT protein may be employed in accordance with the present invention. Accordingly, the terms “homology” or “percent homology” or “identical” or “percent identity” or “percentage identity” or “sequence identity” refer in the context of two or more amino acid sequences to two or more sequences or subsequences that are the same, or that have a specified percentage of amino acids that are the same (preferably at least 40% identity, more preferably at least 45%, 50%, 55%, 60%, 65%, 70%, 75%, 80%, 85%, 90%, 91%, 92%, 93%, 94%, 95%, 96%, 97% or 98% identity, most preferably at least 99% identity) compared to the amino acid sequence of human NUT protein as shown in SEQ ID NO. 2.

As mentioned above, the term “rearrangement in the NUT gene” used herein refers to any rearrangement in the NUT gene that is characteristic for NUT midline carcinoma (NMC). Exemplary “rearrangements in the NUT gene” as well as methods for their detection are known in the art (see, for example, French (2010) J Clin Pathol, loc. cit.). For example, the rearrangement can be or can be caused by a translocation of the NUT gene (or a part or fragment thereof).

One particularly preferred translocation is the t15;19 translocation known in the art. This translocation has resulted in the formation of a fusion gene of NUT, the so called BRD4-NUT fusion gene. Accordingly, rearrangement in the NUT gene which are or are caused/associated by the formation of a BRD4/NUT fusion gene are particularly preferred in context of the present invention. Also envisaged in this context is the formation of a fusion gene comprising a sequence encoding the complete BRD4 gene, and/or one or more parts or fragments thereof and comprising a sequence encoding the complete NUT gene and/or one or more parts or fragments thereof. The exemplary BRD4-NUT fusion protein is composed of the N-terminal of BRD4 (amino acids 1-720 out of 1372) and almost the entire protein sequence of NUT (amino acids 6-1127). The N-terminal of BRD4 includes bromodomains 1 and 2 and other, less well characterized functional domains.

Though the majority of known NMC cases are associated with the formation of a BRD4-NUT fusion gene, further rearrangements in the NUT gene have been described in the art, and the term “NUT variant fusion gene” has been coined in the art to cover the remaining NMC subtypes.

One exemplary “NUT variant fusion gene” is the so called “BRD3-NUT fusion gene”. Accordingly, rearrangements in the NUT gene which are or are caused/associated by the formation of a BRD3/NUT fusion gene are also envisaged in context of the present invention. Again, the formation of a fusion gene comprising a sequence encoding the complete BRD3 gene, and/or one or more parts or fragments thereof and comprising a sequence encoding the complete NUT gene and/or one or more parts or fragments thereof is envisaged herein.

The rearrangements in the NUT gene and optionally mutations/rearrangements/aberrant expression of further genes can be detected by methods known in the art. Such methods are, for example described in French CA, 2010 (NUT midline carcinoma. French CA. Cancer Genet Cytogenet. 2010 November; 203(1):16-20.). A person skilled in the art is in the position to adapt the methods for detecting rearrangements in genes described in the above-mentioned documents to the rearrangements in the NUT gene described herein and further rearrangements in this gene known in the art. A person skilled in the art will readily understand that also rearrangements in said gene not described herein but known in the art or mutations yet to be identified may also be used in the context of the present invention.

Exemplary, non-limiting methods to be used in the detection of rearrangements in the NUT gene are described in WO 2010011700, Haack (2009), loc. cit., French (2010) Cancer Genet Cytogenet (loc. cit.) and French (2010) J Clin Pathol (loc. cit.).

Particularly preferred is diagnosis via in situ hybridisation (FISH, CISH, SISH and the like), since these methods can detect any rearrangement in the NUT gene. However, also detection of a gene product of the above described NUT fusion genes is envisaged using routine techniques like immunohistochemical methods, Northern Blot, Real time PCR and the like. This especially useful in cases where said rearrangement in the NUT gene is reflected in expression of the formed NUT fusion gene, as the expression level of the formed NUT fusion gene may be detected. Such methods are particularly envisaged in the detection of BRD3-NUT transcripts and/or BRD4-NUT transcripts. Also immunohistochemical methods (or other routine methods like Western Blots etc.) may be employed to detect expression products on a protein level. For example, antibodies French (2010) Cancer Genet Cytogenet (loc. cit.) or Haack (2009), loc. cit. describe the use of a diagnostic NUT specific monoclonal antibody, taking advantage of the fact the native protein is not expressed outside of the testis.

Further methods which are useful for detecting mutations or rearrangements are methods for sequencing of nucleic acids (e.g. Sanger di-deoxy sequencing), “next generation” methods, single molecule sequencing, methods enabling detection of variant alleles/mutations, such as Real-time PCR, PCR-RFLP assay (see Cancer Research 59 (1999), 5169-5175), mass-spectrometric genotyping (e.g. MALDI-TOF), HPLC, enzymatic methods and SSPC (single strand conformation polymorphism analysis; see Pathol Int (1996) 46, 801-804).

In particular, such methods may include enzymatic amplification of DNA or cDNA fragments using oligonucleotides specifically hybridizing to exonic or intronic parts of the rearranged NUT gene by PCT. Such amplifications may be carried out in two reactions when employing genomic DNA or even in only a single reaction when employing cDNA. The resulting PCR products may be subjected to either conventional Sanger-based dideoxy nucleotide sequencing methods or employing novel parallel sequencing methods (“next generation sequencing”) such as those marketed by Roche (454 technology), Illumina (Solexa technology) or ABI (Solid technology). Rearrangements or mutations may be identified from sequence reads by comparison with publicly available gene sequence data bases. Alternatively, mutations may be identified by allele-specific incorporation of probes that can either be detected using enzymatic detection reactions, fluorescence, mass spectrometry or others; see Vogeser (2007) Dtsch Arztebl 104 (31-32), A2194-200.

Paraffin-embedded clinical material may be used in the detection of rearrangements in the NUT gene. Detection may comprise a histolopathology review of the sample to be tested to ensure tumour tissue is present. A commercially available Kit to be used in the detection method is the AllPrep DNA/RNA FFPE Kit form Quiagen (Germany). Further kits to be used for detecting rearrangements in the NUT gene are commercially available.

A positive result in the detection method indicates the presence of (a) rearrangement(s) in the NUT gene.

In one embodiment of the present invention, the tumor or cancer cell is not only characterized by the presence of at least one rearrangement in the NUT gene, but also, as a further option, by expression of the NOTCH3 gene. It has been shown in the appended examples that cell lines having a NOTCH3 overexpression in addition to a rearrangement in the NUT gene are particularly susceptible to a CDK9 inhibitor. A nucleic acid sequence of the human NOTCH3 gene and a corresponding amino acid sequence are depicted in SEQ ID NOs: 11 and 12, respectively.

As mentioned above, (a) tumor cell(s)/tumor(s) with (a) rearrangement(s) in the NUT gene and overexpression of the NOTCH3 gene is (are) sensitive to treatment with selective CDK9 inhibitors. Therefore, it is envisaged that (a) tumor cell(s)/tumor(s) with (a) with (a) rearrangement(s) in the NUT gene and, optionally, overexpression of the NOTCH3 gene might be particularly sensitive to treatment with CDK9 inhibitors. Therefore, (a) cell(s), (a) tissue(s) or (a) cell culture selected in accordance with the present method with at least one rearrangement in the NUT gene and overexpression of the NOTCH3 gene might be particularly susceptible to a selective CDK9 inhibitor. Accordingly, treatment of patients with a selective CDK9 inhibitor (the patients suffering from NMC) may be particularly successful in respect of, for example, prognosis or survival rate.

Patient(s) may also be subject to co-therapy/co-treatment with a CDK9 inhibitor and a further compound/drug (e.g. (a) NUT inhibitor(s)). Patients suffering from cancer characterized by the presence of at least one rearrangement in the NUT gene (e.g. NMC) and (a) mutation(s) or overexpression of a further gene (e.g. NOTCH3) may only be treated with a CDK9 inhibitor but not in co-therapy with NOTCH3 inhibitor and a selective CDK9 inhibitor if the patients are known to be resistant to such NOTCH3 inhibitor. Of course, co-therapy/combination therapy to be used in context of the present invention may also comprise radiation therapy, conventional chemotherapy and the like.

The following relates to pharmaceutical compositions and drug combinations. In one embodiment, the present invention relates to a CDK9 inhibitor, such as a selective CDK9 inhibitor, as defined herein for use in treating, ameliorating and/or preventing midline carcinoma, like NUT midline carcinoma (NMC). Accordingly, also the use of a CDK9 inhibitor, such as a selective CDK9 inhibitor, for the preparation of a pharmaceutical composition for the treatment, amelioration and/or prevention of midline carcinoma, like NUT midline carcinoma (NMC), is envisaged in context of the present invention.

The terms “treatment”, “treating” and the like are used herein to generally mean obtaining a desired pharmacological and/or physiological effect. The effect may be prophylactic in terms of completely or partially preventing a disease or symptom thereof and/or may be therapeutic in terms of partially or completely curing a disease and/or adverse effect attributed to the disease. The term “treatment” as used herein covers any treatment of a disease in a subject and includes: (a) preventing a disease related to an insufficient immune response from occurring in a subject which may be predisposed to the disease; (b) inhibiting the disease, i.e. arresting its development; or (c) relieving the disease, i.e. causing regression of the disease.

A “patient” or “subject” for the purposes of the present invention includes both humans and other animals, particularly mammals, and other organisms. Thus, the methods are applicable to both human therapy and veterinary applications. In the preferred embodiment the patient is a mammal, and in the most preferred embodiment the patient is human.

In accordance with the above, the present invention relates to drug combinations and pharmaceutical compositions comprising at least one CDK9 inhibitor, such as (a) compound(s) of general formula (I) as active ingredient together with at least one pharmaceutically acceptable carrier, excipient and/or diluent and optionally together with one or more other anti-tumor agents As used herein the term “drug combination” refers to a combination of at least to pharmaceutically active agents or therapeutic agents with or without further ingredients, carrier, diluents and/or solvents. As used herein the term “pharmaceutical composition” refers to a galenic formulation of at least one pharmaceutically active agent together with at least one further ingredient, carrier, diluent and/or solvent.

CDK9 inhibitors, such as compounds of formula (I) may be administered as the sole pharmaceutical agent or in combination with one or more additional therapeutic agents, wherein the drug combination causes no unacceptable adverse effects. This combination therapy includes administration of a single pharmaceutical dosage formulation, which contains a CDK9 inhibitor and one or more additional therapeutic agents in form of a single pharmaceutical composition, as well as administration of a CDK9 inhibitor and each additional therapeutic agent in its own separate pharmaceutical dosage formulation, i.e. in its own separate pharmaceutical composition. For example, a CDK9 inhibitor and a therapeutic agent may be administered to the patient together in a single oral dosage composition such as a tablet or capsule, or each agent may be administered in separate pharmaceutical compositions.

Where separate pharmaceutical compositions are used, a CDK9 inhibitor and one or more additional therapeutic agents may be administered at essentially the same time (e.g., concurrently) or at separately staggered times (e.g., sequentially).

In particular, the CDK9 inhibitors to be used in accordance with the present invention may be used in fixed or separate pharmaceutical compositions with other anti-tumor agents such as alkylating agents, anti-metabolites, plant-derived anti-tumor agents, hormonal therapy agents, topoisomerase inhibitors, camptothecin derivatives, kinase inhibitors, targeted drugs, antibodies, interferons and/or biological response modifiers, anti-angiogenic compounds, and other anti-tumor drugs. In this regard, the following is a non-limiting list of examples of secondary agents that may be used in combination with the CDK9 inhibitors:

Alkylating agents include, but are not limited to, nitrogen mustard N-oxide, cyclophosphamide, ifosfamide, thiotepa, ranimustine, nimustine, temozolomide, altretamine, apaziquone, brostallicin, bendamustine, carmustine, estramustine, fotemustine, glufosfamide, mafosfamide, and mitolactol; platinum-coordinated alkylating compounds include, but are not limited to, cisplatin, carboplatin, eptaplatin, lobaplatin, nedaplatin, oxaliplatin, and satraplatin;

Anti-metabolites include, but are not limited to, methotrexate, 6-mercaptopurine riboside, mercaptopurine, 5-fluorouracil alone or in combination with leucovorin, tegafur, doxi-fluridine, carmofur, cytarabine, cytarabine ocfosfate, enocitabine, gemcitabine, fludarabin, 5-aza-citidine, capecitabine, cladribine, clofarabine, decitabine, eflornithine, ethynylcytidine, cytosine arabinoside, hydroxyurea, melphalan, nelarabine, nolatrexed, ocfosfite, disodium premetrexed, pentostatin, pelitrexol, raltitrexed, triapine, trimetrexate, vidarabine, vincristine, and vinorelbine;

Hormonal therapy agents include, but are not limited to, exemestane, Lupron, anastrozole, doxercalciferol, fadrozole, formestane, 11-beta hydroxysteroid dehydrogenase 1 inhibitors, 17-alpha hydroxylase/17,20 lyase inhibitors such as abiraterone acetate, 5-alpha reductase inhibitors such as finasteride and epristeride, anti-estrogens such as tamoxifen citrate and fulvestrant, Trelstar, toremifene, raloxifene, lasofoxifene, letrozole, anti-androgens such as bicalutamide, flutamide, mifepristone, nilutamide, Casodex, and anti-progesterones and combinations thereof;

Plant-derived anti-tumor substances include, e.g., those selected from mitotic inhibitors, for example epothilones such as sagopilone, ixabepilone and epothilone B, vinblastine, vinflunine, docetaxel, and paclitaxel;

Cytotoxic topoisomerase inhibiting agents include, but are not limited to, aclarubicin, doxorubicin, amonafide, belotecan, camptothecin, 10-hydroxycamptothecin, 9-aminocamptothecin, diflomotecan, irinotecan, topotecan, edotecarin, epimbicin, etoposide, exatecan, gimatecan, lurtotecan, mitoxantrone, pirambicin, pixantrone, rubitecan, sobuzoxane, tafluposide, and combinations thereof;

Immunologicals include interferons such as interferon alpha, interferon alpha-2a, interferon alpha-2b, interferon beta, interferon gamma-1a and interferon gamma-n1, and other immune enhancing agents such as L19-IL2 and other IL2 derivatives, filgrastim, lentinan, sizofilan, TheraCys, ubenimex, aldesleukin, alemtuzumab, BAM-002, dacarbazine, daclizumab, denileukin, gemtuzumab, ozogamicin, ibritumomab, imiquimod, lenograstim, lentinan, melanoma vaccine (Corixa), molgramostim, sargramostim, tasonermin, tecleukin, thymalasin, tositumomab, Vimlizin, epratuzumab, mitumomab, oregovomab, pemtumomab, and Provenge; Merial melanoma vaccine;

Biological response modifiers are agents that modify defense mechanisms of living organisms or biological responses such as survival, growth or differentiation of tissue cells to direct them to have anti-tumor activity; such agents include, e.g., krestin, lentinan, sizofiran, picibanil, ProMune, and ubenimex;

Anti-angiogenic compounds include, but are not limited to, acitretin, aflibercept, angiostatin, aplidine, asentar, axitinib, recentin, bevacizumab, brivanib alaninat, cilengtide, combretastatin, DAST, endostatin, fenretinide, halofuginone, pazopanib, ranibizumab, rebimastat, removab, revlimid, sorafenib, vatalanib, squalamine, sunitinib, telatinib, thalidomide, ukrain, and vitaxin;

Antibodies include, but are not limited to, trastuzumab, cetuximab, bevacizumab, rituximab, ticilimumab, ipilimumab, lumiliximab, catumaxomab, atacicept, oregovomab, and alemtuzumab;

VEGF inhibitors such as, e.g., sorafenib, DAST, bevacizumab, sunitinib, recentin, axitinib, aflibercept, telatinib, brivanib alaninate, vatalanib, pazopanib, and ranibizumab; Palladia;

EGFR (HER1) inhibitors such as, e.g., cetuximab, panitumumab, vectibix, gefitinib, erlotinib, and Zactima;

HER2 inhibitors such as, e.g., lapatinib, tratuzumab, and pertuzumab;

mTOR inhibitors such as, e.g., temsirolimus, sirolimus/Rapamycin, and everolimus;

c-Met inhibitors;

PI3K and AKT inhibitors;

CDK inhibitors;

Spindle assembly checkpoints inhibitors and targeted anti-mitotic agents such as PLK inhibitors, Aurora inhibitors (e.g. Hesperadin), checkpoint kinase inhibitors, and KSP inhibitors;

HDAC inhibitors such as, e.g., panobinostat, vorinostat, MS275, belinostat, and LBH589;

HSP90 and HSP70 inhibitors;

Proteasome inhibitors such as bortezomib and carfilzomib;

Serinethreonine kinase inhibitors including MEK inhibitors (such as e.g. RDEA 119) and Raf inhibitors such as sorafenib;

Farnesyl transferase inhibitors such as, e.g., tipifarnib;

Tyrosine kinase inhibitors including, e.g., dasatinib, nilotibib, DAST, bosutinib, sorafenib, bevacizumab, sunitinib, AZD2171, axitinib, aflibercept, telatinib, imatinib mesylate, brivanib alaninate, pazopanib, ranibizumab, vatalanib, cetuximab, panitumumab, vectibix, gefitinib, erlotinib, lapatinib, tratuzumab, pertuzumab, and c-Kit inhibitors; Palladia, masitinib;

Vitamin D receptor agonists;

Bcl-2 protein inhibitors such as obatoclax, oblimersen sodium, and gossypol;

Cluster of differentiation 20 receptor antagonists such as, e.g., rituximab;

Ribonucleotide reductase inhibitors such as, e.g., gemcitabine;

Tumor necrosis apoptosis inducing ligand receptor 1 agonists such as, e.g., mapatumumab;

5-Hydroxytryptamine receptor antagonists such as, e.g., rEV598, xaliprode, palonosetron hydrochloride, granisetron, Zindol, and AB-1001;

Integrin inhibitors including alpha5-beta1 integrin inhibitors such as, e.g., E7820, JSM 6425, volociximab, and endostatin;

Androgen receptor antagonists including, e.g., nandrolone decanoate, fluoxymesterone, Android, Prost-aid, andromustine, bicalutamide, flutamide, apo-cyproterone, apo-flutamide, chlormadinone acetate, Androcur, Tabi, cyproterone acetate, and nilutamide;

Aromatase inhibitors such as, e.g., anastrozole, letrozole, testolactone, exemestane, aminoglutethimide, and formestane;

Matrix metalloproteinase inhibitors;

Other anti-cancer agents including, e.g., alitretinoin, ampligen, atrasentan bexarotene, bortezomib, bosentan, calcitriol, exisulind, fotemustine, ibandronic acid, miltefosine, mitoxantrone, 1-asparaginase, procarbazine, dacarbazine, hydroxycarbamide, pegaspargase, pentostatin, tazaroten, velcade, gallium nitrate, canfosfamide, darinaparsin, and tretinoin.

The CDK9 inhibitors may also be employed in cancer treatment in conjunction with radiation therapy and/or surgical intervention.

Furthermore, the CDK9 inhibitors may be utilized, as such or in compositions, in research and diagnostics, or as analytical reference standards, and the like, which are well known in the art.

Thus, another aspect of the present invention relates to drug combinations comprising at least one inventive CDK9 inhibitor, such as a compound according to general formula (I) and/or pharmaceutically acceptable salts thereof together with at least one anti-retroviral drug, especially at least one of the drugs mentioned above.

The pharmaceutical compositions according to the present invention comprise at least one CDK9 inhibitor according to the present invention as an active ingredient together with at least one pharmaceutically acceptable (i.e. non-toxic) carrier, excipient and/or diluent. The pharmaceutical compositions of the present invention can be prepared in a conventional solid or liquid carrier or diluent and a conventional pharmaceutically-made adjuvant at suitable dosage level in a known way. The preferred preparations are adapted for oral application. These administration forms include, for example, pills, tablets, film tablets, coated tablets, capsules, powders and deposits.

Furthermore, the present invention also includes pharmaceutical preparations for parenteral application, including dermal, intradermal, intragastral, intracutan, intravasal, intravenous, intramuscular, intraperitoneal, intranasal, intravaginal, intrabuccal, percutan, rectal, subcutaneous, sublingual, topical, or transdermal application, which preparations in addition to typical vehicles and/or diluents contain at least one CDK9 inhibitor according to the present invention and/or a pharmaceutical acceptable salt thereof as active ingredient.

The pharmaceutical compositions according to the present invention containing at least one CDK9 inhibitor according to the present invention and/or a pharmaceutical acceptable salt thereof as active ingredient will typically be administered together with suitable carrier materials selected with respect to the intended form of administration, i.e. for oral administration in the form of tablets, capsules (either solid filled, semi-solid filled or liquid filled), powders for constitution, gels, elixirs, dispersable granules, syrups, suspensions, and the like, and consistent with conventional pharmaceutical practices. For example, for oral administration in the form of tablets or capsules, the active drug component may be combined with any oral non-toxic pharmaceutically acceptable carrier, preferably with an inert carrier like lactose, starch, sucrose, cellulose, magnesium stearate, dicalcium phosphate, calcium sulfate, talc, mannitol, ethyl alcohol (liquid filled capsules) and the like. Moreover, suitable binders, lubricants, disintegrating agents and coloring agents may also be incorporated into the tablet or capsule. Powders and tablets may contain about 5 to about 95-weight % of the CDK9 inhibitors (such as 2,4,6-disubstituted pyrimidine derivative according to the general formula (I) or analogues compound thereof) or the respective pharmaceutically active salt as active ingredient.

Suitable binders include starch, gelatin, natural sugars, corn sweeteners, natural and synthetic gums such as acacia, sodium alginate, carboxymethylcellulose, polyethylene glycol and waxes. Among suitable lubricants there may be mentioned boric acid, sodium benzoate, sodium acetate, sodium chloride, and the like. Suitable disintegrants include starch, methylcellulose, guar gum, and the like. Sweetening and flavoring agents as well as preservatives may also be included, where appropriate. The disintegrants, diluents, lubricants, binders etc. are discussed in more detail below.

Moreover, the pharmaceutical compositions of the present invention may be formulated in sustained release form to provide the rate controlled release of any one or more of the components or active ingredients to optimise the therapeutic effect(s), e.g. antihistaminic activity and the like. Suitable dosage forms for sustained release include tablets having layers of varying disintegration rates or controlled release polymeric matrices impregnated with the active components and shaped in tablet form or capsules containing such impregnated or encapsulated porous polymeric matrices.

Liquid form preparations include solutions, suspensions, and emulsions. As an example, there may be mentioned water or water/propylene glycol solutions for parenteral injections or addition of sweeteners and opacifiers for oral solutions, suspensions, and emulsions. Liquid form preparations may also include solutions for intranasal administration.

Aerosol preparations suitable for inhalation may include solutions and solids in powder form, which may be present in combination with a pharmaceutically acceptable carrier such as an inert, compressed gas, e.g. nitrogen.

For preparing suppositories, a low melting wax, such as a mixture of fatty acid glycerides like cocoa butter is melted first, and the active ingredient is then dispersed homogeneously therein e.g. by stirring. The molten, homogeneous mixture is then poured into conveniently sized moulds, allowed to cool, and thereby solidified.

Also included are solid form preparations, which are intended to be converted, shortly before use, to liquid form preparations for either oral or parenteral administration. Such liquid forms include solutions, suspensions, and emulsions.

The CDK9 inhibitors according to the present invention may also be delivered transdermally. The transdermal compositions may have the form of a cream, a lotion, an aerosol and/or an emulsion and may be included in a transdermal patch of the matrix or reservoir type as is known in the art for this purpose.

The term capsule as recited herein refers to a specific container or enclosure made e.g. of methylcellulose, polyvinyl alcohols, or denatured gelatins or starch for holding or containing compositions comprising the active ingredient(s). Capsules with hard shells are typically made of blended of relatively high gel strength gelatins from bones or pork skin. The capsule itself may contain small amounts of dyes, opaquing agents, plasticisers and/or preservatives.

Under tablet a compressed or moulded solid dosage form is understood which comprises the active ingredients with suitable diluents. The tablet may be prepared by compression of mixtures or granulations obtained by wet granulation, dry granulation, or by compaction well known to a person of ordinary skill in the art.

Oral gels refer to the active ingredients dispersed or solubilised in a hydrophilic semi-solid matrix.

Powders for constitution refers to powder blends containing the active ingredients and suitable diluents which can be suspended e.g. in water or in juice.

Suitable diluents are substances that usually make up the major portion of the composition or dosage form. Suitable diluents include sugars such as lactose, sucrose, mannitol, and sorbitol, starches derived from wheat, corn, rice, and potato, and celluloses such as microcrystalline cellulose. The amount of diluent in the composition can range from about 5 to about 95% by weight of the total composition, preferably from about 25 to about 75 weight %, and more preferably from about 30 to about 60 weight %.

The term disintegrants refers to materials added to the composition to support break apart (disintegrate) and release the pharmaceutically active ingredients of a medicament. Suitable disintegrants include starches, “cold water soluble” modified starches such as sodium carboxymethyl starch, natural and synthetic gums such as locust bean, karaya, guar, tragacanth and agar, cellulose derivatives such as methylcellulose and sodium carboxymethylcellulose, microcrystalline celluloses, and cross-linked microcrystalline celluloses such as sodium croscaramellose, alginates such as alginic acid and sodium alginate, clays such as bentonites, and effervescent mixtures. The amount of disintegrant in the composition may range from about 2 to about 20 weight % of the composition, more preferably from about 5 to 10 weight %.

Binders are substances which bind or “glue” together powder particles and make them cohesive by forming granules, thus serving as the “adhesive” in the formulation. Binders add cohesive strength already available in the diluent or bulking agent. Suitable binders include sugars such as sucrose, starches derived from wheat, corn, rice and potato, natural gums such as acacia, gelatin and tragacanth, derivatives of seaweed such as alginic acid, sodium alginate and ammonium calcium alginate, cellulose materials such as methylcellulose, sodium carboxymethylcellulose and hydroxypropylmethylcellulose, polyvinylpyrrolidone, and inorganic compounds such as magnesium aluminum silicate. The amount of binder in the composition may range from about 2 to about 20 weight % of the composition, preferably from about 3 to about 10 weight %, and more preferably from about 3 to about 6 weight %.

Lubricants refer to a class of substances which are added to the dosage form to enable the tablet granules etc. after being compressed to release from the mould or die by reducing friction or wear. Suitable lubricants include metallic stearates such as magnesium stearate, calcium stearate, or potassium stearate, stearic acid, high melting point waxes, and other water soluble lubricants such as sodium chloride, sodium benzoate, sodium acetate, sodium oleate, polyethylene glycols and D,L-leucine. Lubricants are usually added at the very last step before compression, since they must be present at the surface of the granules. The amount of lubricant in the composition may range from about 0.2 to about 5 weight % of the composition, preferably from about 0.5 to about 2 weight %, and more preferably from about 0.3 to about 1.5 weight % of the composition.

Glidants are materials that prevent caking of the components of the pharmaceutical composition and improve the flow characteristics of granulate so that flow is smooth and uniform. Suitable glidants include silicon dioxide and talc. The amount of glidant in the composition may range from about 0.1 to about 5 weight % of the final composition, preferably from about 0.5 to about 2 weight %.

Coloring agents are excipients that provide coloration to the composition or the dosage form. Such excipients can include food grade dyes adsorbed onto a suitable adsorbent such as clay or aluminum oxide. The amount of the coloring agent may vary from about 0.1 to about 5 weight % of the composition, preferably from about 0.1 to about 1 weight %.

Embodiments of the present invention is further illustrated by the following non-limiting examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention or the scope of the appended claims.

The present invention is further described by reference to the following non-limiting figures and examples.

### EXAMPLES

The Figures show:

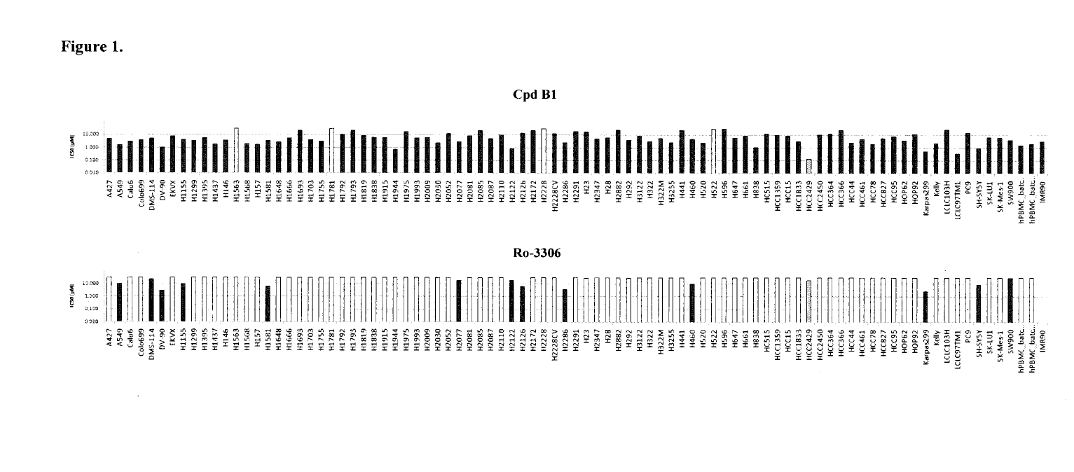

### FIG. 1. Proliferation Inhibition Profile

FIG. 1 shows a proliferation inhibition profile of a potent specific CDK9 inhibitor (Cpd B1) and a selective CDK1 inhibitor (Ro-3306).

Proliferation assays were performed as described below under materials and methods. Three compounds (Cpd B1 and Ro-3306) were applied at concentrations between 30 and 0.0137 μM. After 72 h incubation with compounds ATP content/proliferation was determined employing CTG (Promega). Relative proliferation values (compared to vehicle control) were used to calculate IC₅₀ values (Excel fit; algorithm #205). IC₅₀s of the respective compound (Y-axis; logarithmic scale) on proliferation of various cell lines (X-axis) are depicted in black bars. White bars indicate that an IC₅₀ could not be determined due to too low activity and therefore was higher than the highest applied concentration in the assays (30 μM). IC₅₀s of compounds on CDK9 inhibitor sensitive cell line HCC2429 are presented in grey bars. The IC₅₀s of Cpd B1 was determined at 0.151 μM. The specific CDK1 inhibitor Ro-3306 does not affect said cell line potently (IC₅₀ initially higher 10 μM).

### FIG. 2. CDK9 Inhibition

FIG. 2 shows CDK9 inhibition by and selectivity of described compounds. The figure summarizes IC₅₀ values of 12 compounds on CDK1/CyclinB 1, CDK2/CyclinA, CDK4/CyclinD1, CDK6/CyclinD3, CDK7/CyclinH/Mat1 and CDK9/CyclinT1 activity (methods are described below).

Compounds were either already described (SNS-032: Piperidine-4-carboxylic acid [5-(5-tert-butyl-oxazol-2-ylmethylsulfanyl)-thiazol-2-yl]-amide; Misra R N et al. J Med. Chem. 2004, 47(7): 1719-28; flavopiridol: 2-(2-Chloro-phenyl)-5,7-dihydroxy-8-(3-hydroxy-1-methyl-piperidin-4-yl)-chromen-4-one, Lloyd R Kelland. Expert Opinion on Investigational Drugs. 2000, 9(12):2903-2911; AX35427: N-(5-((6-(3-aminophenyl)pyrimidin-4-yl)amino)-2-methylphenyl)propane-1-sulfonamide, 848637-29-6P in WO 2005026129; R-547: [4-amino-2-(1-methanesulfonylpiperidin-4-ylamino)pyrimidin-5-yl]-(2,3-difluoro-6-methoxyphenyl)methanone, DePinto W et al., Mol Cancer Ther 2006, 5:2644-2658;

3-[[6-(2-methoxyphenyl)-4-pyrimidinyl]amino]-Benzenemethanesulfonamide compound 1073485-20-7P in WO 2008132138.

AX38679: 3-((6-(2-methoxyphenyl)pyrimidin-4-yl)amino)benzenesulfonamide, 848637-62-7P in WO 2005026129; PHA767491: 1,5,6,7-tetrahydro-2-(4-pyridinyl)-4H-pyrrolo[3,2-c]pyridin-4-one, Montagnoli, A. Nat Chem Biol 2008, 4(6) 357-365; BS181: N5-(6-aminohexyl)-3-(1-methylethyl)-N-7-(phenylmethyl), Ali S et al. Cancer Res. 2009, 69(15):6208-15) or mentioned above (Cpd 24, Cpd Cl, Cpd B1 and Cpd B2).

### FIG. 3. CDK9 Inhibitors 1073485-20-7P and Cpd B1.

FIG. 3 shows general kinase selectivity of two typical selective CDK9 inhibitors 1073485-20-7P and Cpd B1. Employing enzymatic in vitro assays on the activity of 23 different kinases incubated with 10 μM Cpd B1 or DMSO as control resulted in the depicted residual activities (compared to the vehicle control). As a result it is demonstrated that Cpd B1 inhibits only CDK9 with high potency and is a selective CDK9 kinase inhibitor in accordance with the present invention.

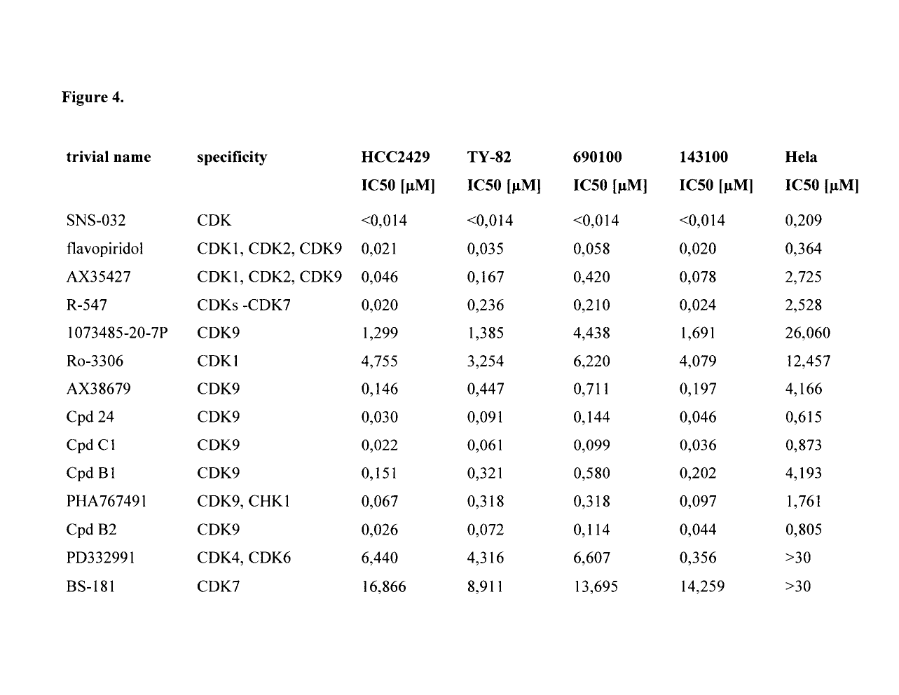

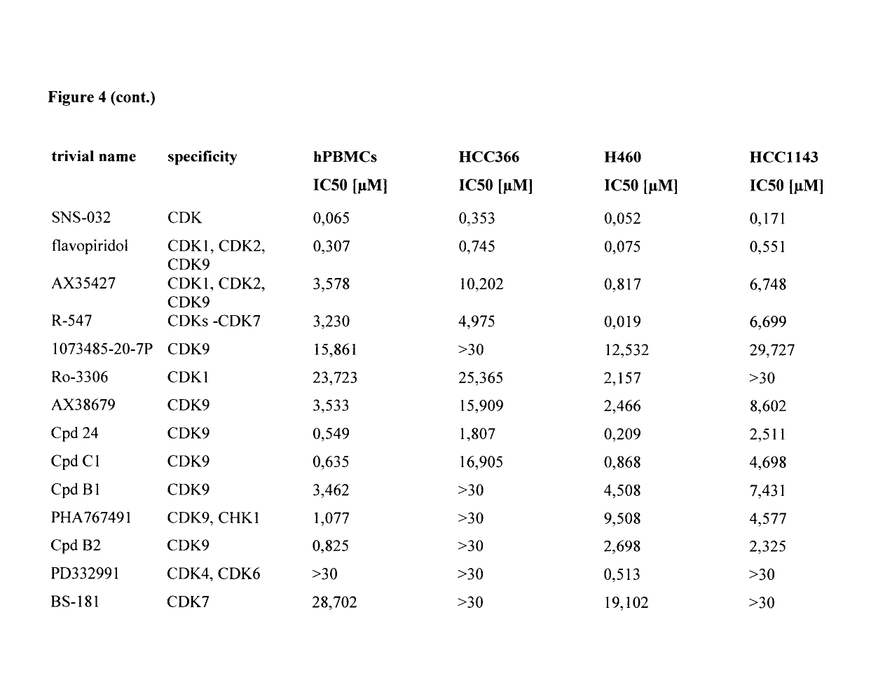

### FIG. 4.

FIG. 4 displays proliferation assay results of selected CDK9 inhibitors as well as other CDK standard inhibitors on various Brd4-Nut mutated as well as wild type cell lines. The proliferation results are presented as IC₅₀ in μM. First, the anti-proliferative effect of CDK9 inhibitors is much more pronounced on Brd/Nut mutated cell lines (=sensitive cell lines). Second, within the table it is clearly visible, that the anti-proliferative effect of the compounds is directly correlated to the Even with relatively weak CDK9 Inhibitors like 1073485-20-7P proliferation inhibition is much more potent in mutated, sensitive cell lines (HCC2429, TY-82, 690100, 143100) than in normal wild type cell lines (Hela, HCC366, H460 HCC1143 and hPBMCs).

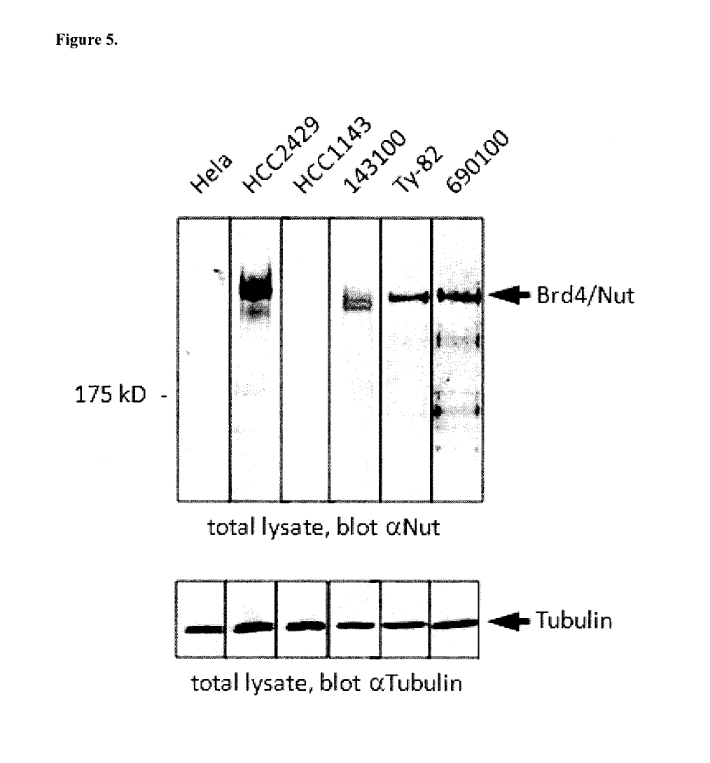

### FIG. 5.

FIG. 5 shows the expression of Brd/Nut fusion proteins in various cell lines (Hela, HCC2429, Ty-82, 143100, 69100 and HCC1143). Cell lysates were analyzed as described in materials and methods. Fusion proteins were detected as high molecular weight bands employing an antibody directed against Nut proteins. As a loading control same lysates were analyzed for their tubulin content.

The Examples illustrate the invention.

### Example 1

### Material and Methods

### Material

NSCLC cells (A427, A549, Calu6, Colo699, DMS-114, DV-90, EKVX, H1155, H1299, H1395, H1437, H146, H1563, H1568, H157, H1581, H1648, H1666, H1693, H1703, H1755, H1781, H1792, H1793, H1819, H1838, H1915, H1944, H1975, H1993, H2009, H2030, H2052, H2077, H2081, H2085, H2087, H2110, H2122, H2126, H2172, H2228, H2228CV, H2286, H2291, H23, H2347, H28, H2882, H292, H3122, H322, H322M, H3255, H441, H460, H520, H522, H596, H647, H661, H838, HC515, HCC1359, HCC15, HCC1143, HCC1833, HCC2429, HCC2450, HCC364, HCC366, HCC44, HCC461, HCC78, HCC827, HCC95, HOP62, HOP92, Karpas299, Kelly, LCLC103H, LCLC97TM1, PC9, SH-SY5Y, SK-LU1, SK-Mes-1, SW900, 690100 and 143100) are described in PCT patent application WO 2010020618 and WO 2010020619. TY-82 cells were purchased from Health Science Research Resource Bank (Osaka, Japan). Peripheral blood mononuclear cells (hPBMCs) were provided by the Blutspendedienst Hagen (DRK West, Hagen). All other cell lines (e.g. Hela cells) have been purchased from LGC Standards (ATCC, Wesel) or the DSMZ (Deutsche Sammlung von Mikroorganismen and Zellkulturen, Braunschweig).

### Cell Viability Assays

Cell lines were maintained in RPMI 1640 cell culture medium+glutamine (PAN Biotech GmbH, Aidenbach, Germany; order no. P04-22100; P04-05500) supplemented with 10% fetal calf serum “Gold” (PAA Laboratories GmbH, Pasching, Austria; order no. A15-151) and grown in a humidified atmosphere at 37° C., 5% CO2. As a first step, an optimal cell density for each cell line was determined to guarantee linearity. For viability assays with compounds, cells were then seeded at a density of 200 to 1000 per well in 25 μA in 384-well plates (Greiner Bio-One, Frickenhausen, Germany; order no. 781080). After overnight incubation at 37° C./5% CO2, 25 nl or 75 nl compounds were added to each sample well by using BIOMEK FX^{P} Laboratory Automation Workstation (Beckman Coulter, USA). Wells with cells and 0.1% or 0.3% DMSO in culture medium were used as positive controls, wells with cells and 10 μM staurosporine (Selleck Chemicals, Huston, USA) in culture medium were used as negative controls. Upon incubation with compounds for 72 h at 37° C./5% CO2 25 μl Cell Titer Glo reagent (Promega, Madison, USA, order no. G7573; 1:2 diluted with cell culture medium) was added to each well to determine cell viability. 384 well-plates were placed for 2 min on an orbital microplate shaker and incubated for further 10 min at room temperature resulting in a stabilization of light signal. Luminescence was measured by Envision Plate Reader (Perkin Elmer, USA). IC50 values were calculated with the software Excel Fit (IDBS, Guildford, UK) from 3-fold dilution series comprising 8 concentrations in duplicates.

### Immunoblot Analysis

Cellular proteins were solubilized in CLB-A buffer (Zeptosens, Switzerland). After addition of 3×Lämmli buffer (30% v/v glycerol, 6% SDS, 150 mM Tris/HCl [pH 6.8], 0.3% w/v bromophenol blue, 300 mM DTT) proteins were denatured by incubation at 95° C. for 5 min. Thereon, proteins were separated on SDS-PAGE and transferred to PVDF membranes (Immobilon®, Milipore, Schwalbach). After blocking, bound proteins were incubated with antibodies against [symbol] bulin (T59840R, Biozol; clone B-5-1-2, Sigma-Aldrich) or Nut (C52B1, Cell Signaling, Frankfurt a. M.) diluted in blocking buffer (Li-Cor biosciences, Bad Homburg, Germany).

### Measurement of Half Maximal Inhibitory Concentration to CDKs

This protocol describes how the Lance Ultra KinaSelect Assay was performed to determine half maximal inhibitory concentration (IC₅₀) of compounds of general formula (I) and CDK/Cyclin complexes. The principle behind this enzymatic assay is based upon the phosphorylation of the Ulight-Peptide Substrate. It is detected by using a specific EU-labeled anti-phospho peptide antibody. The binding of the Eu labeled anti-phospho peptide antibody to the phosphorylated ULight labeled peptide gives rise to a FRET-signal. Binding of an inhibitor to the kinase prevents phosphorylation of the Ulight-MBP Substrate, resulting in a loss of FRET.

TABLE 3
Reagents, stock concentrations and final assay concentrations

| | | Kinase- | ATP- | | | Substrat- | | | Antibody- |
|---|---|---|---|---|---|---|---|---|---|
| Kinase | Supplier | conc. [nM] | conc. [μM] | Substrat | Supplier | conc. [nM] | Antibody | Supplier | conc. [nM] |
| CDK1/CyclinB1 | Carna | 2 | 20 | Ulight MBP | Perkin Elmer | 50 | Eu-anti-P-MBP | Perkin Elmer | 0.25 |
| (91 kDa) | | | | | | | | | |
| CDK2/CyclinA | Proqinase | 5 | 3 | Ulight MBP | Perkin Elmer | 50 | Eu-anti-P-MBP | Perkin Elmer | 0.25 |
| (135 kDa) | | | | | | | | | |
| CDK4/CyclinD1 | Invitrogen | 10 | 90 | Ulight MBP | Perkin Elmer | 50 | Eu-anti-P-MBP | Perkin Elmer | 0.25 |
| (123 kDa) | | | | | | | | | |
| CDK6/CyclinD3 | Carna | 5 | 55 | Ulight MBP | Perkin Elmer | 50 | Eu-anti-P-MBP | Perkin Elmer | 0.025 |
| (123 kDa) | | | | | | | | | |
| CDK7/CyclinH/ | Invitrogen | 10 | 25 | Ulight MBP | Perkin Elmer | 50 | Eu-anti-P-MBP | Perkin Elmer | 0.25 |
| MAT1(126 kDa) | | | | | | | | | |
| CDK9/CyclinT1 | Invitrogen | 10 | 25 | Ulight MBP | Perkin Elmer | 50 | Eu-anti-P-MBP | Perkin Elmer | 0.25 |
| (132 kDa) | | | | | | | | | |
| CDK9/CyclinK | Invitrogen | 10 | 125 | Ulight MBP | Perkin Elmer | 50 | Eu-anti-P-MBP | Perkin Elmer | 0.25 |
| (92 kDa) | | | | | | | | | |

The selective CDK9 inhibitors described herein above (see also Table 1 and Table 2) were diluted from a 10 mM DMSO stock solution 1:10 in a total volume of 15 μl DMSO. This compound predilution was then serial diluted 1:3 over 8 steps in DMSO and briefly spun down. Each compound solution was now diluted 1:20 in Enzymatic Buffer (HEPES: 50 mM, pH: 7.5; MgCl₂: 10 mM; EGTA: 1 mM; DTT: 2 mM; Tween-20: 0.01%), mixed thoroughly and spun down. For every sample, 2 μl of the diluted compound were mixed with 6 μl CDK/Cyclin/Substrate solution and 2 μl ATP solution in a well of a small volume 384 well plate (Corning Incorporated, Corning, N.Y., USA; order no. 3673). The CDK/Cyclin was diluted to the appropriate concentration (see Table 3 and the ATP concentration was adjusted to its IC₅₀ concentration for the CDK/Cyclin, which was 3 μM for CDK2 Cyclin A, 20 μM for CDK1/Cyclin B1, 25 μM CDK7/Cyclin H and CDK9/Cyclin Ti, 55 μM CDK6/Cyclin D3, 90 μM CDK4/Cyclin D1 and 125 μM for CDK9/Cyclin K. For negative controls, in each well 2 μA of DMSO solution (1% final DMSO assay concentration) was mixed with 6 μA substrate solution (50 nM Ulight MBP final assay concentration) and 2 μA ATP solution (appropriate final concentration see Table 3. For positive controls, in each well 2 μA of DMSO solution (1% final DMSO assay concentration) was mixed with 6 μA CDK/Cyclin/Substrate (appropriate final concentration see Table 3 and 2 μA Tracer ATP solution (appropriate final concentration see Table 3. Positive and negative controls were calculated from at least 8 different sample wells. The 384 well plates were mixed in a Teleshaker plate mixer (Beckman Coulter, Brea, Calif., USA) at 2000 rpm for 40 sec, and incubated for 1 h at room temperature. Before reading, 10 μl the detection buffer (Lance Detection Buffer 1×; EDTA: 20 nM; Eu-Anti-P-MBP: see Table 3 was added. The FRET signal was measured at 340 nm excitation, 665 nm and 615 nm emission (for the kinase tracer and LanthaScreen Eu-AB, respectively) with an Envision spectrophotometer (Perkin Elmer, Waltham, Mass., USA) with 50 μs delay and 300 μs integration time. IC₅₀ values were determined from the sigmoidal dose response curves with the software Quattro Workflow (Quattro GmbH, Munich, Germany).

### Other Kinase Assays:

A radiometric protein kinase assay (33 PanQinase® Activity Assay) was used for measuring the kinase activity of the 333 protein kinases. All kinase assays were performed in 96-well FlashPlates™ from Perkin Elmer (Boston, Mass., USA) in a 50 μl reaction volume. The reaction cocktail was pipetted in 4 steps in the following order:

10 μl of non-radioactive ATP solution (in H2O)

25 μl of assay buffer/[γ-33P]-ATP mixture

5 μl of test sample in 10% DMSO

10 μl of enzyme/substrate mixture

The assay for all enzymes contained 70 mM HEPES-NaOH, pH 7.5, 3 mM MgCl₂, 3 mM MnCl2, 3 μM Na-orthovanadate, 1.2 mM DTT, ATP/[γ-33P]-ATP (variable amounts, corresponding to the apparent ATP-Km of the respective kinase, see Table 4 below approx. 8×1005 cpm per well), protein kinase (variable amounts; see Table 4), and substrate (variable amounts; see Table 4). All protein kinases provided by ProQinase were expressed in Sf9 insect cells or in E. coli as recombinant GST-fusion proteins or His-tagged proteins. All kinases were produced from human cDNAs. Kinases were purified by affinity chromatography using either GSH-agarose or Ni-NTA-agarose. The purity of the protein kinases was examined by SDS-PAGE/Coomassie staining. The identity of the protein kinases was checked by mass spectroscopy. The concentrations of enzymes and substrates used for the assays are shown in Table 4 below.

The reaction cocktails were incubated at 30° C. for 60 minutes. The reaction was stopped with 50 μl of 2% (v/v) H3PO4, plates were aspirated and washed two times with 200 μl 0.9% (w/v) NaCl. All assays were performed with a BeckmanCoulter Biomek 2000/SL robotic system. Incorporation of 33Pi (counting of “cpm”) was determined with a microplate scintillation counter (Microbeta, Wallac).

TABLE 4
Conditions of additional kinase assays

| Kinase | | | | | | | | |
|---|---|---|---|---|---|---|---|---|
| | | External | Kinase | | ATP | | | |
| | PQ | Vendor | Conc. | | Conc. | Substrate | | |
| Name | Lot | Lot | ng/50 μl | nM* | μM | Name | Lot | μg/50 μl |
| ABL1 wt | 5 | | 25 | 6.6 | 0.3 | Poly(Ala,Glu,Lys,Tyr)6:2:5:1 | SIG_53H5516 | 0.125 |
| AKT1 | 7 | | 25 | 6.4 | 3 | GSK3(14-27) | 11 | 2 |
| Aurora-A | 4 | | 50 | 13.1 | 10 | tetra(LRRWSLG) | 6 | 0.5 |
| CDK9/CycT | 4 | | 15 | 1.9 | 1 | RBER-CHKtide | 25 | 1 |
| CK2-alpha1 | 3 | | 20 | 5.6 | 0.1 | Casein | SIG_83K7430 | 1 |
| EGF-R wt | 17 | | 20 | 4.5 | 0.3 | Poly(Glu,Tyr)4:1 | SIG_20K5903 | 0.125 |
| EPHB4 | 7 | | 10 | 2.6 | 1 | Poly(Glu,Tyr)4:1 | SIG_20K5903 | 0.125 |
| FGF-R1 | 1 | INV_31517 | 3 | 1.2 | 1 | Poly(Glu,Tyr)4:1 | SIG_20K5903 | 0.125 |
| FLT3 wt | 11 | | 25 | 6.1 | 3 | Poly(Ala,Glu,Lys,Tyr)6:2:5:1 | SIG_53H5516 | 0.125 |
| GSK3-beta | 3 | | 50 | 13.1 | 0.3 | RBER-CHKtide | 24 | 1 |
| IGF1-R | 12 | | 10 | 2.6 | 1 | Poly(Glu,Tyr)4:1 | SIG_20K5903 | 0.125 |
| IKK-beta | 8 | | 50 | 8.3 | 0.3 | RBER-CHKtide | 24 | 2 |
| JNK1 | 5 | | 5 | 2.3 | 0.3 | ATF2 | 11 | 2 |
| LCK | 3 | | 20 | 4.3 | 0.3 | Poly(Glu,Tyr)4:1 | SIG_20K5903 | 0.125 |
| MAPKAPK3 | 1 | | 10 | 4.6 | 0.1 | tetra(LRRWSLG) | 6 | 0.25 |
| MEK1 | 2 | 50 | 23 | 3 | | ERK2 K54R (kinase-dead) | 6 | 2 |
| MET wt | 12 | | 20 | 5.1 | 0.3 | Poly(Ala,Glu,Lys,Tyr)6:2:5:1 | SIG_53H5516 | 0.125 |
| PDGFR-beta | 13 | | 50 | 11.4 | 0.3 | Poly(Ala,Glu,Lys,Tyr)6:2:5:1 | SIG_53H5516 | 0.125 |
| PLK1 | 13 | | 15 | 3.0 | 1 | RBER-CHKtide | 25 | 2 |
| p38-alpha | 5 | | 10 | 4.8 | 1 | ATF2 | 10 | 2 |
| ROCK2 | 2 | | 5 | 1.1 | 0.3 | S6-Peptide | 6 | 1 |
| TGFB-R1 | 3 | | 5 | 1.6 | 1 | GSK3(14-27) | 9 | 1 |
| VEGF-R2 | 15 | | 25 | 5.7 | 1 | Poly(Glu,Tyr)4:1 | SIG_20K5903 | 0.125 |

### Results

Within a screening approach to profile more than 1600 kinase inhibitors and other compounds on proliferation of 86 cell lines we identified HCC2429 cells to be sensitive for CDK9 inhibitors (specific as well as unspecific). This initial finding was verified by dose response experiments (see FIG. 1). In these experiments selective CDK9 inhibitors (Cpd B1) potently affected proliferation of HCC2429 cells whereas a specific CDK1 inhibitor (Ro-3306) did not show comparable effects (FIG. 1). HCC2429 cells overexpress Notch3 and are known to contain a t(15, 19) translocation. The later one results in the expression of a Brd4/Nut fusion protein. We confirmed the selectivity of a set of additional compounds by in vitro kinase assays (FIG. 2). To this end we performed assays for CDKs as well as for other kinases (ABL1 wt, AKT1, Aurora-A, CDK1/CycA, CDK1/CycE, CDK2/CycA, CDK3/CycE, CDK4/CycD1, CDK6/CycD1, CDK7/CycH/MAT1, CDK9/CycT, CK2-alpha1, EGF-R wt, EPHB4, FGF-R1 wt, FLT3 wt, GSK3-beta, IGF1-R, IKK-beta, JNK1, LCK, MAPKAPK3, MEK1 wt, MET, PDGFR-beta, PLK1, p38-alpha, ROCK2, TGFB-R1, VEGF-R2). Altogether these experiments corroborate the enormous selectivity of the pyridine, triazine and pyrimidine compounds. Profiling of selected CDK9 inhibitors on cells bearing a similar t(15, 19) translocation (Ty-82, 690100, 143100 together with HCC2429) or control cells (HCC1143, H3122, PC9, A549, Hela, H460 and hPBMCs) confirmed that this rearrangement sensitizes for CDK9 inhibition (FIG. 4). Interestingly, overexpression of Notch3 did not result in sensitization as shown by Notch3 overexpressing cell lines HCC1143 and others (H1793, H1819, H441 and H647; data not shown). The expression of the fusion gene was confirmed in said cells by western blot analysis (FIG. 5).

The present invention refers to the following nucleotide and amino acid sequences:

The sequences provided herein are available in the NCBI database and can be retrieved from www.ncbi.nlm.nih.gov/sites/entrez?db=gene; Theses sequences also relate to annotated and modified sequences. The present invention also provides techniques and methods wherein homologous sequences, and also genetic allelic variants and the like of the concise sequences provided herein are used. Preferably, such “variants” are genetic variants.

### SEQ ID No. 1:

Nucleotide sequence encoding homo sapiens nut gene (alias Homo sapiens chromosome 15 open reading frame 55 (C15orf55); accession number NM_{—}175741.1):

The coding region ranges from nucleotide 156 to nucleotide 3554.

### SEQ ID No. 2:

Amino acid sequence of homo sapiens Nut protein:

### SEQ ID No. 3:

Nucleotide sequence encoding homo sapiens brd4 (accession number NM_{—}058243.2):

The coding region ranges from nucleotide 223 to nucleotide 4311.

### SEQ ID No. 4:

Amino acid sequence of homo sapiens Brd4 protein:

### SEQ ID No. 5:

Nucleotide sequence encoding homo sapiens brd3 (accession number NM_{—}007371.3).

The coding region ranges from nucleotide 189 to nucleotide 2369.

### SEQ ID No. 6:

Amino acid sequence of homo sapiens Brd3:

### SEQ ID No. 7:

Nucleotide sequence encoding homo sapiens cdk9 (accession number NM_{—}001261.3, alias TAK, C-2k, CTK1, CDC2L4, PITALRE):

The coding region ranges from nucleotide 124 to nucleotide 1242.

### SEQ ID No. 8:

Amino acid sequence of homo sapiens CDK9:

### SEQ ID No. 9:

Nucleotide sequence encoding homo sapiens cyclinT1 (accession number NM_{—}001240.2):

The coding region ranges from nucleotide 324 to nucleotide 2504.

### SEQ ID No. 10:

Amino acid sequence of homo sapiens CyclinT1:

### SEQ ID No. 11:

Nucleotide sequence encoding homo sapiens Notch3.

The coding region ranges from nucleotide 77 to nucleotide 7042.

### SEQ ID No. 12:

Amino acid sequence of homo sapiens Notch3:

All references cited herein are fully incorporated by reference. Having now fully described the invention, it will be understood by a person skilled in the art that the invention may be practiced within a wide and equivalent range of conditions, parameters and the like, without affecting the spirit or scope of the invention or any embodiment thereof.

What is claimed is:

1. A CDK9 inhibitor for use in treating, ameliorating and/or preventing midline carcinoma.

2. A method for treating, preventing or ameliorating midline carcinoma comprising the administration of a CDK9 inhibitor to a subject in need of such a treatment, prevention or amelioration.

3. The CDK9 inhibitor for use in treating, ameliorating and/or preventing midline carcinoma according to claim 1, or the method for treating, preventing or ameliorating midline carcinoma according to claim 2, wherein said midline carcinoma is NUT midline carcinoma (NMC).

4. The method for treating, preventing or ameliorating midline carcinoma according to claim 2, wherein said CDK9 inhibitor is a selective CDK9 inhibitor.

5. The method according to claim 4, wherein said selective CDK9 inhibitor is a compound having the general formula (I) 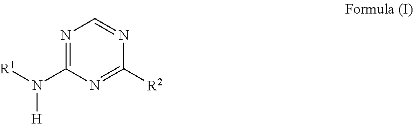
wherein
R¹ is 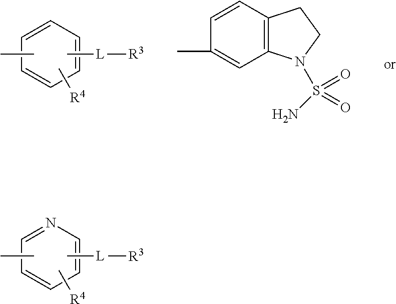
L is a bond or —CR⁵R⁶—, —CR⁵R⁶—CR⁷R⁸—, —CR⁵R⁶—CR⁷R⁸—CR⁹R¹⁰—, —CR⁵R⁶—CR⁷R⁸—CR⁹R¹⁰—CR¹¹R¹²—;
R⁵-R¹² represent independently of each other —H, —CH₃, —C₂H₅, —C₃H₇, —F, —Cl, —Br, —I;
R³ is selected from the group consisting of —H, —NO₂, —NH₂, —CN, —F, —Cl, —Br, —I, —CH₃, —C₂H₅, -Ph, —C₃H₇, —CH(CH₃)₂, —C₄H₉, —CH₂—CH(CH₃)₂, —CH(CH₃)—C₂H₅, —C(CH₃)₃, —O—CH₃, —O—C₂H₅, —O—C₃H₇, —O—CH(CH₃)₂, —O—C₄H₉, —O—CH₂CH₃)₂, —O—CH(CH₃)—C₂H₅, —O—C(CH₃)₃, —CR¹³R¹⁴R²¹, —CR¹³R¹⁴—CR¹⁵R¹⁶R²¹, —O—CR¹³R¹⁴R²¹, —CR¹³R¹⁴—CR¹⁵R¹⁶—CR¹⁷R¹⁸R²¹, —CR¹³R¹⁴—CR¹⁵R¹⁶—CR¹⁷R¹⁸—CR¹⁹R²⁰R²¹, —O—CR¹³R¹⁴—CR¹⁵R¹⁶R²¹, —O—CR¹³R¹⁴—CR¹⁵R¹⁶—CR¹⁷R¹⁸R²¹, —SO₂R²², —CONR²³R²⁴, —NR²⁵COR²², —O—CR¹³R¹⁴—CR¹⁵R¹⁶—CR¹⁷R¹⁸—CR¹⁹R²⁰R²¹, NR²⁵SO₂NR²³R²⁴, —NR²⁵SO₂R²², —NR²⁵CONR²³R²⁴, —SO₂NR²³R²⁴, —SO(NR²⁶)R²⁷, —NH—CO—NH-Ph;
R¹³-R²¹, R²⁹-R³² and R³³-R⁴⁸ represent independently of each other —H, —F, —Cl, —Br, —I;
R²⁶ is —H, —CH₃, —C₂H₅, —C₃H₇, —CH(CH₃)₂, —C₄H₉, —CH₂—CH(CH₃)₂, —CH(CH₃)—C₂H₅, —C(CH₃)₃, —C₅H₁₁, —CH(CH₃)—C₃H₇, —CH₂—CH(CH₃)—C₂H₅, —CH(CH₃)—CH(CH₃)₂, —C(CH₃)₂—C₂H₅, —CH₂—C(CH₃)₃, —CH(C₂H₅)₂, —C₂H₄—CH(CH₃)₂, —C₆H₁₃, —C₃H₆—CH(CH₃)₂, —C₂H₄—CH(CH₃)—C₂H₅, —CH(CH₃)—C₄H₉, —CH₂—CH(CH₃)—C₃H₇, —CH(CH₃)—CH₂—CH(CH₃)₂, —CH(CH₃)—CH(CH₃)—C₂H₅, —CH₂—CH(CH₃)—CH(CH₃)₂, —CH₂—C(CH₃)₂—C₂H₅, —C(CH₃)₂—C₃H₇, —C(CH₃)₂—CH(CH₃)₂, —C₂H₄—C(CH₃)₃, —CH(CH₃)—C(CH₃)₃, —CR¹³R¹⁴R²¹, —COR²⁸, —CR¹³R¹⁴—CR¹⁵R¹⁶R²¹, —CR¹³R¹⁴—CR¹⁵R¹⁶—CR¹⁷R¹⁸—CR¹⁹R²⁰—CR²⁹R³⁰R²¹, —CR¹³R¹⁴—CR¹⁵R¹⁶—CR¹⁷R¹⁸R²¹, —CR¹³R¹⁴—CR¹⁵R¹⁶—CR¹⁷R¹⁸—CR¹⁹R²⁰R²¹, —CR¹³R¹⁴—CR¹⁵R¹⁶—CR¹⁷R¹⁸—CR¹⁹R²⁰—CR²⁹R³⁰—CR³¹R³²R²¹, —COOR²⁸, 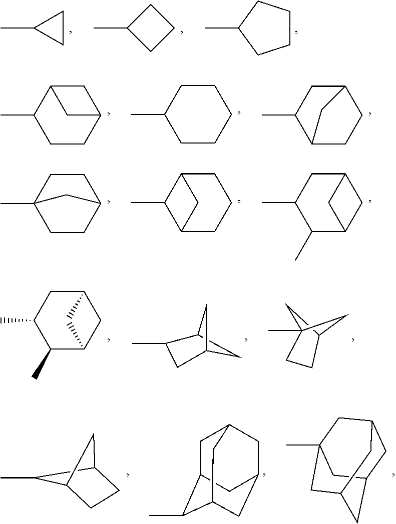
these C₃-C₆-cycloalkyl groups may further be substituted by one, two, three, four, five or more substituents selected from the group consisting of R³³-R⁴⁸;
R²², R²⁷, and R²⁸ are independently selected from —CR⁴⁹R⁵⁰R⁵¹, —CR⁴⁹R⁵⁰—CR⁵²R⁵³R⁵¹, —CR⁴⁹R⁵⁰—CR⁵²R⁵³—CR⁵⁴R⁵⁵—CR⁵⁶R⁵⁷—CR⁵⁸R⁵⁹R⁵¹, —CR⁴⁹R⁵⁰—CR⁵²R⁵³—CR⁵⁴R⁵⁵R⁵¹, CR⁴⁹R⁵⁰—CR⁵²R⁵³, —CR⁵⁴R⁵⁵—CR⁵⁶R⁵⁷R⁵¹, —CR⁴⁹R⁵⁰—CR⁵²R⁵³—CR⁵⁴R⁵⁵—CR⁵⁶R⁵⁷—CR⁵⁸R⁵⁹—CR⁶⁰R⁶¹R⁵¹, —CH₂Ph, —CH₂Ph the phenyl group of which may further be substituted by one, two, three, four or five substituents selected from the group consisting of R⁵-R¹²; C₃-C₆-cycloalkyl groups listed for R²⁶, which may further be substituted by one, two, three, four, five or more substituents selected from the group consisting of R³³-R⁴⁸;
R⁴⁹-R⁶¹ represent independently of each other —H, —CH₃, —C₂H₅, —C₃H₇, —C₄H₉, —F, —Cl, —Br, —I, —OH, —NO₂, —NH₂;
R²³ and R²⁴ are independently selected from —H, —CR⁴⁹R⁵⁰R⁵¹, —CR⁴⁹R⁵⁰—CR⁵²R⁵³R⁵¹, —CR⁴⁹R⁵⁰—CR⁵²R⁵³—CR⁵⁴R⁵⁵—CR⁵⁶R⁵⁷—CR⁵⁸R⁵⁹R⁵¹, —CR⁴⁹R⁵⁰—CR⁵²R⁵³—CR⁵⁴R⁵⁵R⁵¹, —CR⁴⁹R⁵⁰—CR⁵²R⁵³—CR⁵⁴R⁵⁵—CR⁵⁶R⁵⁷R⁵¹, —CR⁴⁹R⁵⁰—CR⁵²R⁵³—CR⁵⁴R⁵⁵—CR⁵⁶R⁵⁷—CR⁵⁸R⁵⁹—CR⁶⁰R⁶¹R⁵¹, —CR⁴⁹R⁵⁰—CR⁵²R⁵³—O—R^{51′}, —CR⁴⁹R⁵⁰—CR⁵²R⁵³—CR⁵⁴R⁵⁵—O—R^{51′}, —CR⁴⁹R⁵⁰—CR⁵²R⁵³—NR^{51′}R^{51″}, —CR⁴⁹R⁵⁰—CR⁵²R⁵³—CR⁵⁴R⁵⁵—NR^{51′}R^{51″}, —CR⁴⁹R⁵⁰—CR⁵²R⁵³—CR⁵⁴R⁵⁵—CR⁵⁶R⁵⁷—NR^{51′}R^{51″}, —CR⁴⁹R⁵⁰—CR⁵²R⁵³—CR⁵⁴R⁵⁵—CR⁵⁶R⁵⁷—CR⁵⁸R⁵⁹—NR^{51′}R^{51″}, phenyl, substituted phenyl, benzyl, substituted benzyl, or both residues R²³ and R²⁴ together form with the nitrogen atom to which they are attached a azetidine, pyrrolidine, piperidine, piperazine, azepane, or morpholine ring;
R^{51′} and R^{51″} represent independently of each other —H, —CH₃, —C₂H₅, —C₃H₇, —C₄H₉, —CH₂Ph, —COOC(CH₃)₃, —COOCH₃, —COOCH₂CH₃, —COOCH₂CH₂CH₃, —COOCH(CH₃)₂, —COOCH₂Ph, —COCH₃;
and R²⁵ is selected from —H, —CH₃, —C₂H₅, —C₃H₇, —CH(CH₃)₂, —C₄H₉, —CH₂—CH(CH₃)₂, —CH(CH₃)—C₂H₅ or —C(CH₃)₃;
R⁴ is selected from —H, —NO₂, —NH₂, —CN, —F, —Cl, —Br, —I, —CR⁶²R⁶³R⁶⁴, 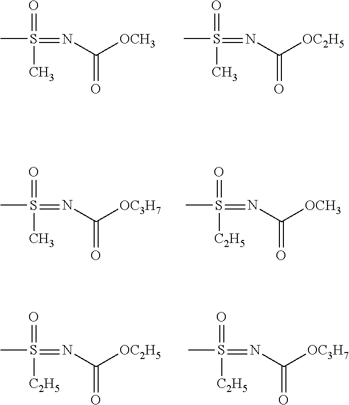
—CONH₂, —SO₂CH₃, —SO₂C₂H₅, —SO₂C₃H₇, —NH—SO₂—CH₃, —NH—SO₂—C₂H₅, —NH—SO₂—C₃H₇, —NHCO—CH₃, —NHCO—C₂H₅, —NHCO—C₃H₇, —SO₂NR²³R²⁴, —CH₂—SO₂NR²³R²⁴, —C₂H₄—SO₂NR²³R²⁴, —C₃H₆—SO₂NR²³R²⁴, —SO₂NH₂, —CH₂—SO₂NH₂, —C₂H₄—SO₂NH₂, —C₃H₆—SO₂NH₂, —O—CR⁶²R⁶³—CR⁶⁵R⁶⁶R⁶⁴, —O—CR⁶²R⁶³—CR⁶⁵R⁶⁶—CR⁶⁷R⁶⁸R⁶⁴, —CR⁶²R⁶³—CR⁶⁵R⁶⁶—CR⁶⁷R⁶⁸—CR⁶⁹R⁷⁰R⁶⁴, —O—CR⁶²R⁶³—CR⁶⁵R⁶⁶—CR⁶⁷R⁶⁸—CR⁶⁹R⁷⁰R⁶⁴, CR⁶²R⁶³—CR⁶⁵R⁶⁶—CR⁶⁷R⁶⁸R⁶⁴, —O—CR⁶²R⁶³—CR⁶⁵R⁶⁶—CR⁶⁷R⁶⁸—CR⁶⁹R⁷⁰—CR⁷¹R⁷²R⁶⁴, —CR⁶²R⁶³, —CR⁶⁵R⁶⁶R⁶⁴, —O—CR⁶²R⁶³—CR⁶⁵R⁶⁶—CR⁶⁷R⁶⁸—CR⁶⁹R⁷⁰—CR⁷¹R⁷²—CR⁷³R⁷⁴R⁶⁴, —O—CR⁶²R⁶³R⁶⁴, —CR⁶²R⁶³—CR⁶⁵R⁶⁶—CR⁶⁷R⁶⁸—CR⁶⁹R⁷⁰—CR⁷¹R⁷²R⁶⁴, —CR⁶²R⁶³—CR⁶⁵R⁶⁶—CR⁶⁷R⁶⁸—CR⁶⁹R⁷⁰—CR⁷¹R⁷²—CR⁷³R⁷⁴R⁶⁴, —OCH₂Ph, 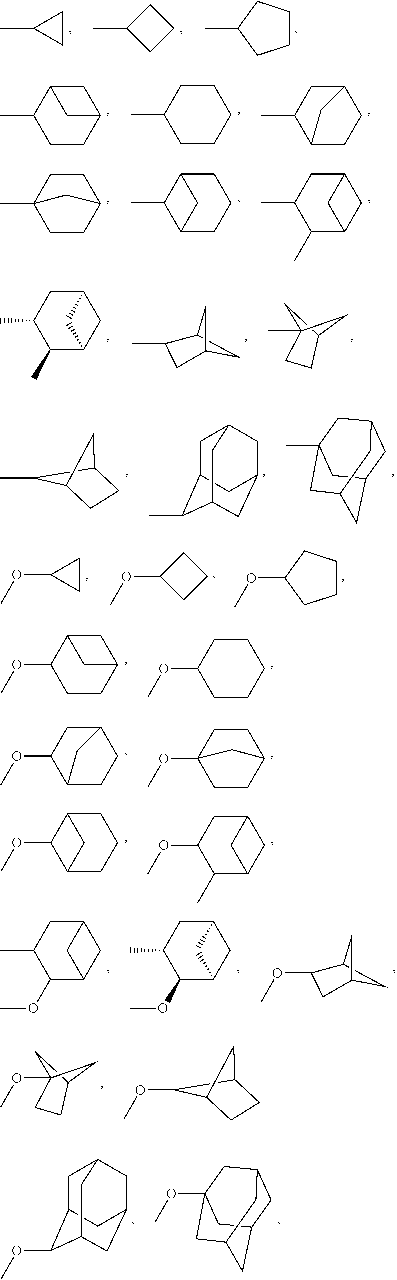
these C₃-C₆-cycloalkoxy groups and C₃-C₆-cycloalkyl groups may further be substituted by one, two, three, four, five or more substituents selected from the group consisting of R³³-R⁴⁸;
R⁶²-R⁷⁴ represent independently of each other —H, cyclo-C₃H₅, cyclo-C₄H₇, -cyclo-C₅H₉, —CR⁷⁵R⁷⁶R⁷⁷, —CR⁷⁵R⁷⁶—CR⁷⁸R⁷⁹R⁷⁷, —CR⁷⁵R⁷⁶—CR⁷⁸R⁷⁹—CR⁸⁰R⁸¹R⁷⁷, —CR⁷⁵R⁷⁶—CR⁷⁸R⁷⁹—CR⁸⁰R⁷⁹—CR⁸²R⁸¹R⁷⁷, —F, —Cl, —Br, —I, -Ph;
R⁷⁵-R⁸² represent independently of each other —H, —F, —Cl, —Br, —I, —NH₂;
R⁴ together with R²², R²³, R²⁴, or R²⁵ may form a group —CH₂CH₂— or —CH₂CH₂CH₂— if R⁴ is attached ortho to -L-R³;
R² is 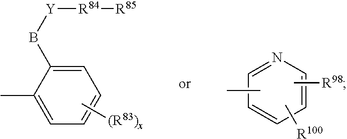
R⁸³ is selected from —H, —OH, —NO₂, —CN, —F, —Cl, —Br, —I, —CF₃, —NR^{23′}R^{24′}, —CR⁶²R⁶³R⁶⁴, —CR⁶²R⁶³—NR^{23′}R^{24′}, —CR⁶²R⁶³—CR⁶⁵R⁶⁶R⁶⁴, —CR⁶²R⁶³—CR⁶⁵R⁶⁶—NR^{23′}R^{24′}, —CR⁶²R⁶³—CR⁶⁵R⁶⁶—CR⁶⁷R⁶⁸R⁶⁴, —CR⁶²R⁶³—CR⁶⁵R⁶⁶—CR⁶⁷R⁶⁸—NR^{23′}R^{24′}, —O—CR⁶²R⁶³R⁶⁴, —O—CR⁶²R⁶³—CR⁶⁵R⁶⁶R⁶⁴, —O—CR⁶²R⁶³—CR⁶⁵R⁶⁶—CR⁶⁷R⁶⁸R⁶⁴, —CHO, —CH₂OH, —CR^{23′}O, CH₂OR^{23′};
R^{23′} and R^{24′} represent independently of each other —H, —CH₃, —C₂H₅, —C₃H₇, —CH(CH₃)₂, —C₄H₉, —CH₂—CH(CH₃)₂, —CH(CH₃)—C₂H₅, —C(CH₃)₃; -(cyclo-C₃H₅);
x is a value between 0 and 3;
B is a bond, —CR⁸⁶R⁸⁷—, —CR⁸⁶R⁸⁷—CR⁸⁸R⁸⁹—, —CR⁸⁶R⁸⁷—CR⁸⁸R⁸⁹—CR⁹⁰R⁹¹—, —CR⁸⁶R⁸⁷—CR⁸⁸R⁸⁹—CR⁹⁰R⁹¹—CR⁹²R⁹³—, —CR⁸⁶R⁸⁷—CR⁸⁸R⁸⁹—CR⁹⁰R⁹¹—CR⁹²R⁹³—CR⁹⁴R⁹⁵—, —CR⁸⁶R⁸⁷—CR⁸⁸R⁸⁹—CR⁹⁰R⁹¹—CR⁹²R⁹³—CR⁹⁴R⁹⁵—CR⁹⁶R⁹⁷—;
R⁸⁶-R⁹⁷ represent independently of each other —H, —CH₃, —C₂H₅, —C₃H₇, —C₄H₉, —F, —Cl, —Br, —I;
Y is a bond, —O—, —S—, —SO—, —SO₂—, —SO₂NH—, —NHSO₂—, —CO—, —COO—, —OOC—, —CONH—, —NHCO—, —NH—, —N(CH₃)—, —NH—CO—NH—, —O—CO—NH—, —NH—CO—O—;
R⁸⁴ is selected from a bond, —CR⁸⁶R⁸⁷—, —CR⁸⁶R⁸⁷—CR⁸⁸R⁸⁹—CR⁹⁰R⁹¹—, —CR⁸⁶R⁸⁷—CR⁸⁸R⁸⁹—CR⁹⁰R⁹¹—CR⁹²R⁹³—, —CR⁸⁶R⁸⁷—CR⁸⁸R⁸⁹—CR⁹⁰R⁹¹—CR⁹²R⁹³—CR⁹⁴R⁹⁵—, —CR⁸⁶R⁸⁷—CR⁸⁸R⁸⁹—, —CR⁸⁶R⁸⁷—CR⁸⁸R⁸⁹—CR⁹⁰R⁹¹—CR⁹²R⁹³—CR⁹⁴R⁹⁵—CR⁹⁶R⁹⁷—;
R⁸⁵ is selected from
(i) —H, —OH, —OCH₃, —OC₂H₅, —OC₃H₇, —O-cyclo-C₃H₅, —OCH(CH₃)₂, —OC(CH₃)₃, —OC₄H₉, -Ph, —OPh, —OCH₂-Ph, —OCPh₃, —SH, —SCH₃, —SC₂H₅, —SC₃H₇, —S-cyclo-C₃H₅, —SCH(CH₃)₂, —SC(CH₃)₃, —SC₄H₉, —NO₂, —F, —Cl, —Br, —I, —P(O)(OH)₂, —P(O)(OCH₃)₂, —P(O)(OC₂H₅)₂, —P(O)(OCH(CH₃)₂)₂, —Si(CH₃)₂(C(CH₃)₃), —Si(C₂H₅)₃, —Si(CH₃)₃, —CN, —CHO, —COCH₃, —COC₂H₅, —COC₃H₇, —CO-cyclo-C₃H₅, —COCH(CH₃)₂, —COC(CH₃)₃, —COC₄H₉, —COOH, —COOCH₃, —COOC₂H₅, —COOC₃H₇, —COOC₄H₉, —COO-cyclo-C₃H₅, —COOCH(CH₃)₂, —COOC(CH₃)₃, —OOC—CH₃, —OOC—C₂H₅, —OOC—C₃H₇, —OOC—C₄H₉, —OOC-cyclo-C₃H₅, —OOC—CH(CH₃)₂, —OOC—C(CH₃)₃, —CONR^{23′}R^{24′}, —NHCOCH₃, —NHCOC₂H₅, —NHCOC₃H₇, —NHCO-cyclo-C₃H₅, —NHCO—CH(CH₃)₂, —NHCOC₄H₉, —NHCO—C(CH₃)₃, —NHCO—OCH₃, —NHCO—OC₂H₅, —NHCO—OC₃H₇, —NHCO—O-cyclo-C₃H₅, —NHCO—OC₄H₉, —NHCO—OCH(CH₃)₂, —NHCO—OC(CH₃)₃, —NHCO—OCH₂Ph, —NR²³R²⁴, —CF₃, —SOCH₃, —SOC₂H₅, —SOC₃H₇, —SO-cyclo-C₃H₅, —SOCH(CH₃)₂, —SOC(CH₃)₃, —SO₂CH₃, —SO₂C₂H₅, —SO₂C₃H₇, —SO₂-cyclo-C₃H₅, —SO₂CH(CH₃)₂, —SO₂C₄H₉, —SO₂C(CH₃)₃, —SO₃H, —SO₂NR^{23′}R^{24′}, —OCF₃, —OC₂F₅, —O—COOCH₃, —O—COOC₂H₅, —O—COOC₃H₇, —O—COO-cyclo-C₃H₅, —O—COOC₄H₉, —O—COOCH(CH₃)₂, —O—COOCH₂Ph, —O—COOC(CH₃)₃, —NH—CO—NH₂, —NH—CO—NHCH₃, —NH—CO—NHC₂H₅, —NH—CO—NHC₃H₇, —NH—CO—NHC₄H₉, —NH—CO—NH-cyclo-C₃H₅, —OCH₂-cyclo-C₃H₅, —NH—CO—NH[CH(CH₃)₂], —NH—CO—NH[C(CH₃)₃], —NH—CO—N(CH₃)₂, —NH—CO—N(C₂H₅)₂, —NH—CO—N(C₃H₇)₂, —NH—CO—N(C₄H₉)₂, —NH—CO—N(cyclo-C₃H₅)₂, —NH—CO—N[CH(CH₃)₂]₂, —NH—CO—N[C(CH₃)₃]₂, —NH—C(═NH)—NH₂, —NH—C(═NH)—NHCH₃, —NH—C(═NH)—NHC₂H₅, —NH—C(═NH)—NHC₃H₇, —NH—C(═NH)—NHC₄H₉, —NH—C(═NH)—NH-cyclo-C₃H₅, —NH—C(═NH)—NH[CH(CH₃)₂], —NH—C(═NH)—NH[C(CH₃)₃], —NH—C(═NH)—N(CH₃)₂, —NH—C(═NH)—N(C₂H₅)₂, —NH—C(═NH)—N(C₃H₇)₂, —NH—C(═NH)—N(cyclo-C₃H₅)₂, —NH—C(═NH)—N(C₄H₉)₂, —NH—C(═NH)—N[CH(CH₃)₂]₂, —NH—C(═NH)—N[C(CH₃)₃]₂, —O—CO—NH₂, —O—CO—NHCH₃, —O—CO—NHC₂H₅, —O—CO—NHC₃H₇, —O—CO—NHC₄H₉, —O—CO—NH-cyclo-C₃H₅, —O—CO—NH[CH(CH₃)₂], —O—CO—NH[C(CH₃)₃], —O—CO—N(CH₃)₂, —O—CO—N(C₂H₅)₂, —O—CO—N(C₃H₇)₂, —O—CO—N(C₄H₉)₂, —O—CO—N(cyclo-C₃H₅)₂, —O—CO—N[CH(CH₃)₂]₂, —O—CO—N[C(CH₃)₃]₂,
(ii) an aromatic or heteroaromatic mono- or bicyclic ring selected from 2-thienyl, 3-thienyl, 2-furanyl, 3-furanyl, 2-oxazolyl, 3-oxazolyl, 4-oxazolyl, 2-thiazolyl, 3-thiazolyl, 4-thiazolyl, 1-pyrazolyl, 3-pyrazolyl, 4-pyrazolyl, 5-pyrazolyl, 1-imidazolyl, 2-imidazolyl, 4-imidazolyl, 5-imidazolyl, phenyl, 1-naphthyl, 2-naphthyl, 2-pyridyl, 3-pyridyl, 4-pyridyl, 2-pyrimidinyl, 4-pyrimidinyl, 5-pyrimidinyl, 2-pyrazinyl, 3-pyridazinyl, 4-pyridazinyl, 1,3,5-triazin-2-yl, 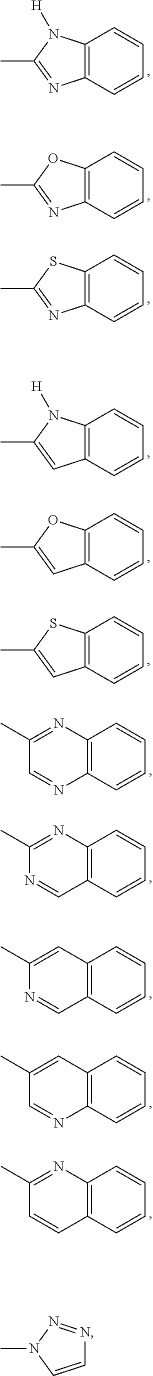 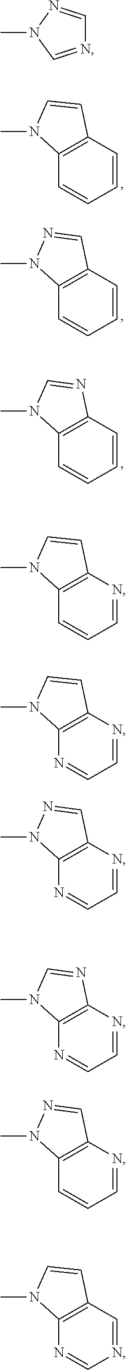 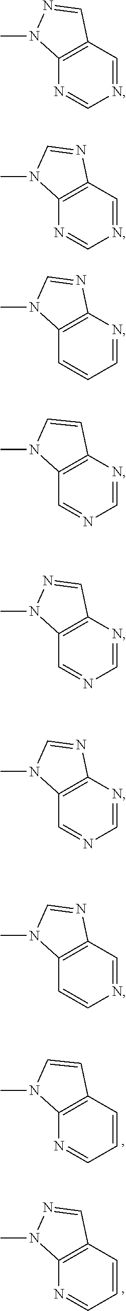 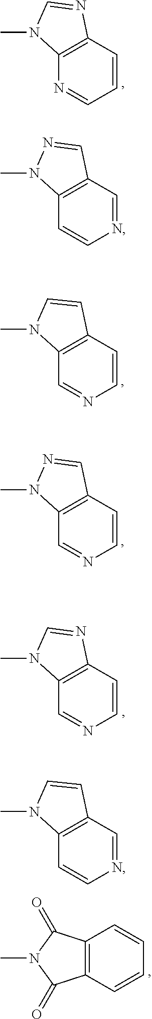
which optionally may be substituted by one or two substituents selected from —F, —Cl, —Br, —I, —OCH₃, —CH₃, —NO₂, —CN, —CF₃;
(iii) a saturated ring selected from cyclopentyl, azetidin-1-yl, 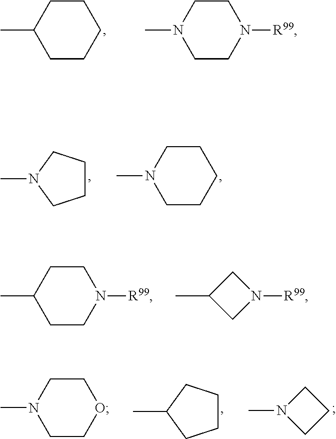
R⁹⁹ represents —H, —CH₃, —CH₂Ph, —COOC(CH₃)₃, —COOCH₃, —COOCH₂CH₃, —COOCH₂CH₂CH₃, —COOCH(CH₃)₂, —COOCH₂Ph, —COCH₃;
the group —B—Y—R⁸⁴—R⁸⁵ together with one substituent R⁸³ may form a group —OCH₂O—, if R⁸³ is attached in position ortho to —B—Y—R⁸⁴—R⁸⁵;
with the proviso that R⁸³ is not —H, if the group —B—Y—R⁸⁴—R⁸⁵ is hydrogen.
R⁹⁸ is selected from —NO₂, —CN, —F, —Cl, —Br, —I, —NH₂, —OH, —CR⁶²R⁶³R⁶⁴, —CR⁶²R⁶³—CR⁶⁵R⁶⁶R⁶⁴, —CR⁶²R⁶³—CR⁶⁵R⁶⁶—CR⁶⁷R⁶⁸R⁶⁴, —CR⁶²R⁶³—CR⁶⁵R⁶⁶—CR⁶⁷R⁶⁸—CR⁶⁹R⁷⁰R⁶⁴, —O—CR⁶²R⁶³R⁶⁴, —O—CR⁶²R⁶³—CR⁶⁵R⁶⁶R⁶⁴, —O—CR⁶²R⁶³—CR⁶⁵R⁶⁶—CR⁶⁷R⁶⁸R⁶⁴, —O—CR⁶²R⁶³—CR⁶⁵R⁶⁶—CR⁶⁷R⁶⁸—CR⁶⁹R⁷⁰R⁶⁴, —O—CR⁶²R⁶³—CR⁶⁵R⁶⁶—CR⁶⁷R⁶⁸—CR⁶⁹R⁷⁰—CR⁷¹R⁷²R⁶⁴, —O—CR⁶²R⁶³—CR⁶⁵R⁶⁶—CR⁶⁷R⁶⁸—CR⁶⁹R⁷⁰—CR⁷¹R⁷²—CR⁷³R⁷⁴R⁶⁴, —CR⁶²R⁶³—O—CR⁶⁵R⁶⁶R⁶⁴, —CR⁶²R⁶³—O—CR⁶⁵R⁶⁶—CR⁶⁷R⁶⁸R⁶⁴, —CR⁶²R⁶³—O—CR⁶⁵R⁶⁶—CR⁶⁷R⁶⁸—CR⁶⁹R⁷⁰R⁶⁴, —CR⁶²R⁶³—O—CR⁶⁵R⁶⁶—CR⁶⁷R⁶⁸—CR⁶⁹R⁷⁰—CR⁷¹R⁷²R⁶⁴, —CR⁶²R⁶³—O—CR⁶⁵R⁶⁶—CR⁶⁷R⁶⁸—CR⁶⁹R⁷⁰—CR⁷¹R⁷²—CR⁷³R⁷⁴R⁶⁴, —CR⁶²R⁶³—CR⁶⁵R⁶⁶—CR⁶⁷R⁶⁸—CR⁶⁹R⁷⁰—CR⁷¹R⁷²R⁶⁴, —CR⁶²R⁶³—CR⁶⁵R⁶⁶—CR⁶⁷R⁶⁸—CR⁶⁹R⁷⁰—CR⁷¹R⁷²—CR⁷³R⁷⁴R⁶⁴, —OCH₂Ph, —OCH₂—CH₂-Ph, —CH₂—O—CH₂-Ph,
with the proviso that R⁹⁸ is attached to a position ortho to the bond between the pyridine and the triazine ring if R⁹⁸ is not an amino group in para position to the bond between the pyridine and the triazine ring;
R¹⁰⁰ is selected from —H, —NO₂, —CN, —F, —Cl, —Br, —I, —NH₂, —OH, —CF₃, —CH₃, —CH₂CH₃, —CH₂CH₂CH₃, —CH(CH₃)₂, —OCH₃, —OCH₂CH₃, —OCH₂CH₂CH₃, —OCH(CH₃)₂, —OCF₃, —OCH₂Ph;
and with the proviso that if R¹ is a phenyl moiety and R² is also a phenyl moiety a chloro substituent is only allowed on the R¹ phenyl moiety or on the R² phenyl moiety but not on both simultaneously;
and with the proviso that the compound 4-[4-(2-benzoylaminophenyl)-[1,3,5]triazin-2-ylamino]benzamide is excluded;
and enantiomers, stereoisomeric forms, mixtures of enantiomers, diastereomers, mixtures of diastereomers, prodrugs, hydrates, solvates, acid salt forms, tautomers, and racemates of the above mentioned compounds and pharmaceutically acceptable salts or salts of solvates thereof.

6. The method according to claim 5, wherein
R¹ represents 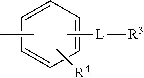
in which
L is a bond, —CH₂—, —CH₂CH₂—, or —CF₂—;
R³ is —SO₂NH₂, —SO₂NH(CH₃), —SO₂N(CH₃)₂, —SO₂NH(CH₂CH₂OCH₃), —NHSO₂CH₃, —NHSO₂CH₂CH₃, —NHSO₂CH₂CH₂CH₃, —NHSO₂CF₃, —SO₂CH₃, —NHSO₂NH₂, —SO(NH)CH₃;
R⁴ is —H, —CH₃, —F, —Cl, or —CF₃;
R² represents 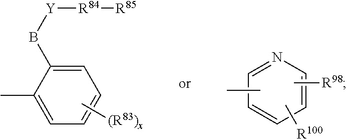
in which the group —B—Y—R⁸⁴—R⁸⁵ is —OCH₃, —OCH₂CH₃, —OCH₂CH₂CH₃, —OCH₂CH₂CH₂CH₃, —OCH(CH₃)₂, —OPh, —OCH₂Ph, —OCH₂(4-pyridyl);
R⁸³ is —H, —F, or —Cl;
x is 0, 1, or 2;
R⁹⁸ is OCH₃ and R¹⁰⁰ is —H,
with the proviso that R⁹⁸ is attached to a position ortho to the bond between the pyridine and the triazine ring.

7. The method according to claim 5, wherein
R¹ represents 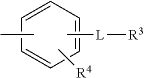
in which
the substituent -L-R³ is —SO₂NH₂, —CH₂SO₂NH₂, —CH₂CH₂SO₂NH₂, —CF₂SO₂NH₂, —NHSO₂NH₂, —CH₂NHSO₂NH₂, —SO₂CH₃, —SO(NH)CH₃, —CH₂SO(NH)CH₃;
R⁴ is —H;
R² represents 2-methoxyphenyl, 4-fluoro-2-methoxyphenyl, or 6-fluoro-2-methoxyphenyl.

8. The method according to claim 5, wherein
R¹ is 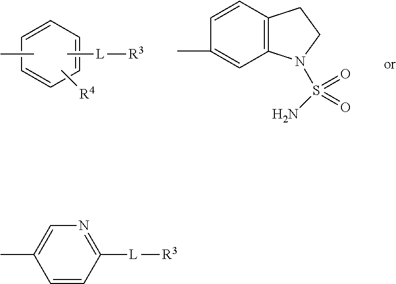
L is a bond, —CH₂—, or —CH₂CH₂—;
R³ is —H, —SO₂NR²³R²⁴, —CONR²³R²⁴, —NO₂, —NH₂, —NHSO₂R²², —NHCOR²², —SO₂R²², —NH—CO—NH-Ph, or -Ph,
R⁴ is —H, —CH₂—SO₂NR²³R²⁴, —SO₂NR²³R²⁴, —CONH₂, —C₂H₄—SO₂NR²³R²⁴, —NH—SO₂—CH₃, —NH—SO₂—C₂H₅, —NH—SO₂—C₃H₇, —NHCO—CH₃, —NHCO—C₂H₅, —NO₂, —NH₂, —SO₂CH₃, or 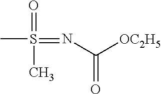
R²³ and R²⁴ are independently selected from the group consisting of —H, —CH₃, —C₂H₅, —C₃H₇, (cyclo-C₃H₅), CH₂CH₂CH₂CH₂NH₂, or CH₂CH₂CH₂CH₂NH COOC(CH₃)₃,
R² represents 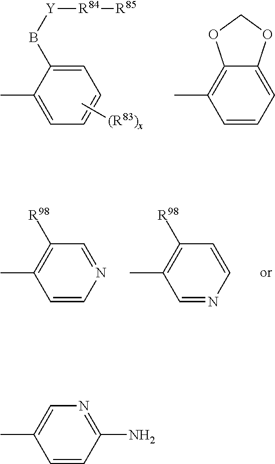
B is a bond or —CH₂—;
Y is a bond, —O—, or —NH—;
R⁸³ is selected from the group consisting of —H, —CN, —F, —Cl, O—CR⁶²R⁶³R⁶⁴, —CF₃, —CH₂OR^{23′}, CR^{23′}O, —CR⁶²R⁶³NR^{23′}R^{24′}, —CR⁶²R²³R⁶⁴;
R^{23′} and R^{24′} represent independently of each other —H, —CH₃, (cyclo-C₃H₅);
R⁶²-R⁶⁴ represent independently of each other —H, —CH₃, -Ph, —F, cyclo-C₃H₅;
R⁸⁴ is selected from a bond, —CH₂—, or —CH₂—CH₂—CH₂—CH₂—;
R⁸⁵ is selected from the group consisting of —H, —CF₃, —OCH₃, —OCH(CH₃)₂, —CN, —NHCOCH₃, —OCH₂-cyclo-C₃H₅, —NR²³R²⁴, -Ph, —OPh, —NHCO—OC(CH₃)₃, 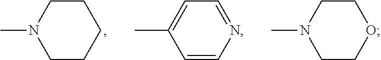
R⁹⁸ represents OCH₃;
and salts, solvates or salts of solvates, hydrochloride salt, or the trifluoroacetate salt of the aforementioned compounds.

9. The method according to claim 5, wherein
R¹ represents 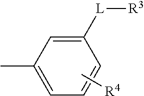
in which
the substituent -L-R³ is —SO₂NH₂ or —CH₂SO₂NH₂,
R⁴ is H;
R² represents 2-methoxyphenyl, 4-fluoro-2-methoxyphenyl or 2-benzyloxyphenyl,
or their salts, solvates or salts of solvates and especially the hydrochloride salt or the trifluoroacetate salt.

10. The method according to claim 5, wherein the compound is selected from the group consisting of
3-[(4-(2-Methoxyphenyl)-1,3,5-triazin-2-yl)amino]benzenemethanesulfonamide,
3-[(4-(4-Fluoro-2-methoxyphenyl)-1,3,5-triazin-2-yl)amino]benzenemethane-sulfonamide,
3-[(4-(5-Fluoro-2-methoxyphenyl)-1,3,5-triazin-2-yl)amino]benzenemethane-sulfonamide,
3-[(4-(6-Fluoro-2-methoxyphenyl)-1,3,5-triazin-2-yl)amino]benzenemethane-sulfonamide,
3-[(4-(3,5-Difluoro-2-methoxyphenyl)-1,3,5-triazin-2-yl)amino]benzenemethane-sulfonamide,
3-[(4-(4-Chloro-2-methoxyphenyl)-1,3,5-triazin-2-yl)amino]benzenemethane-sulfonamide,
3-[(4-(5-Chloro-2-methoxyphenyl)-1,3,5-triazin-2-yl)amino]benzenemethane-sulfonamide,
3-[(4-(2-Methoxy-4-trifluoromethyl-phenyl)-1,3,5-triazin-2-yl)amino]benzene-methanesulfonamide,
3-[(4-(2-Methoxy-5-trifluoromethyl-phenyl)-1,3,5-triazin-2-yl)amino]benzene-methanesulfonamide,
3-[(4-(5-Hydroxymethyl-2-methoxyphenyl)-1,3,5-triazin-2-yl)amino]benzene-methanesulfonamide,
3-[(4-(5-Formyl-2-methoxyphenyl)-1,3,5-triazin-2-yl)amino]benzenemethane-sulfonamide,
3-[(4-(2-Ethoxyphenyl)-1,3,5-triazin-2-yl)amino]benzenemethanesulfonamide,
3-[(4-(2-Benzyloxyphenyl)-1,3,5-triazin-2-yl)amino]benzenemethanesulfonamide,
1-(3-{[4-(2-phenoxyphenyl)-1,3,5-triazin-2-yl]amino}phenyl)methanesulfonamide,
3-[(4-(1,3-Benzodioxol-4-yl)-1,3,5-triazin-2-yl)amino]benzenemethanesulfonamide,
3-[(4-(2-((4-Pyridinyl)methoxy)phenyl)-1,3,5-triazin-2-yl)amino]benzenemethane-sulfonamide,
3-[(4-(2-(4-(tert-Butoxycarbonylamino)butoxy)phenyl)-1,3,5-triazin-2-yl)amino]-benzenemethanesulfonamide,
3-[(4-(4-Methoxypyridin-3-yl)-1,3,5-triazin-2-yl)amino]benzenemethane-sulfonamide,
3-[(4-(3-Methoxypyridin-4-yl)-1,3,5-triazin-2-yl)amino]benzenemethane-sulfonamide,
3-[(4-(2-((Morpholin-4-yl)methyl)phenyl)-1,3,5-triazin-2-yl)amino]benzene-methanesulfonamide,
3-[(4-(2-((Piperidin-1-yl)methyl)phenyl)-1,3,5-triazin-2-yl)amino]benzene-methanesulfonamide,
3-[(4-(2-(Cyclopropylamino-methyl)phenyl)-1,3,5-triazin-2-yl)amino]benzene-methanesulfonamide,
3-[(4-(6-Aminopyridin-3-yl)-1,3,5-triazin-2-yl)amino]benzenemethane-sulfonamide,
3-[(4-(2-(Methoxymethyl)phenyl)-1,3,5-triazin-2-yl)amino]benzene-methanesulfonamide,
3-[(4-(2-Methoxyphenyl)-1,3,5-triazin-2-yl)amino]benzenesulfonamide,
2-[3-((4-(2-Methoxyphenyl)-1,3,5-triazin-2-yl)amino)phenyl]ethanesulfonamide,
2-[3-((4-(4-Fluoro-2-methoxyphenyl)-1,3,5-triazin-2-yl)amino)phenyl]ethane-sulfonamide,
3-[(4-(2-Methoxyphenyl)-1,3,5-triazin-2-yl)amino]benz amide,
6-[(4-(2-Methoxyphenyl)-1,3,5-triazin-2-yl)amino]-2,3-dihydro-1H-indole-1-sulfonamide,
rac-S-[3-((4-(2-Methoxyphenyl)-1,3,5-triazin-2-yl)amino)phenyl]-N-ethoxy-carbonyl-S-methyl-sulfoximide,
4-(2-Methoxyphenyl)-N-(3-nitrophenyl)-1,3,5-triazine-2-amine,
3-[(4-(2-(4-Aminobutoxy)phenyl)-1,3,5-triazin-2-yl)amino]benzenemethane-sulfonamide,
N-(3-Aminophenyl)-4-(2-methoxyphenyl)-1,3,5-triazine-2-amine,
N-[3-((4-(2-Methoxyphenyl)-1,3,5-triazin-2-yl)amino)phenyl]-methanesulfonamide,
N-[3-((4-(2-Methoxyphenyl)-1,3,5-triazin-2-yl)amino)phenyl]-propanesulfonamide,
N-[3-((4-(2-Methoxyphenyl)-1,3,5-triazin-2-yl)amino)phenyl]acetamide,
N-[3-((4-(2-Methoxyphenyl)-1,3,5-triazin-2-yl)amino)phenyl]-N′-phenyl-urea,
3-[(4-(2-Methoxy-5-(methylamino-methyl)phenyl)-1,3,5-triazin-2-yl)amino]-benzenemethanesulfonamide,
4-(2-Methoxyphenyl)-N-phenyl-1,3,5-triazine-2-amine,
tert-Butyl-[4-((3-((4-(4-Fluoro-2-methoxyphenyl)-1,3,5-triazin-2-yl)amino)phenyl)-methylsulfonamido)butyl]carbamate,
N-(4-Aminobutyl)-1-[3-((4-(4-fluoro-2-methoxyphenyl)-1,3,5-triazin-2-yl)amino)-phenyl]methanesulfonamide,
4-(2-Methoxyphenyl)-N-(3-(methylsulfonyl)phenyl)-1,3,5-triazin-2-amine,
4-[(4-(2-Methoxyphenyl)-1,3,5-triazin-2-yl)amino]benzenemethane-sulfonamide,
1-[3-({4-[4-fluoro-2-(trifluoromethyl)phenyl]-1,3,5-triazin-2-yl}amino)phenyl]-methanesulfonamide,
1-[3-({4-[4-fluoro-2-(propan-2-yloxy)phenyl]-1,3,5-triazin-2-yl}amino)phenyl]-methanesulfonamide,
1-(3-{[4-(2-cyano-4-fluorophenyl)-1,3,5-triazin-2-yl]amino}phenyl)methane-sulfonamide,
N-[5-fluoro-2-(4-{[3-(sulfamoylmethyl)phenyl]amino}-1,3,5-triazin-2-yl)phenyl]-acetamide,
1-[3-({4-[2-(cyclopropylmethoxy)-4-fluorophenyl]-1,3,5-triazin-2-yl}amino)phenyl]-methanesulfonamide,
1-(3-{[4-(3,4-difluoro-2-methoxyphenyl)-1,3,5-triazin-2-yl]amino}phenyl)methane-sulfonamide,
1-(3-{[4-(4,5-difluoro-2-methoxyphenyl)-1,3,5-triazin-2-yl]amino}phenyl)methane-sulfonamide,
4-(4-fluoro-2-methoxyphenyl)-N-[6-(methylsulfonyl)pyridin-3-yl]-1,3,5-triazin-2-amine,
3-[(4-(2-Methoxyphenyl)-1,3,5-triazin-2-yl)amino]benzenemethanesulfonamide trifluoroacetic acid salt,
1-(3-{[4-(4-fluoro-2-methoxyphenyl)-1,3,5-triazin-2-yl]amino}phenyl)methane-sulfonamide hydrochloride,
3-[(4-(4-Fluoro-2-methoxyphenyl)-1,3,5-triazin-2-yl)amino]benzene-methanesulfonamide trifluoroacetic acid salt,
3-[(4-(2-Benzyloxyphenyl)-1,3,5-triazin-2-yl)amino]benzenemethanesulfon-amide trifluoroacetic acid salt,
3-[(4-(2-Methoxyphenyl)-1,3,5-triazin-2-yl)amino]benzenesulfonamide trifluoroacetic acid salt.

11. The method according to claim 2, wherein said inhibitor is selected from the group consisting of,
3-[(4-(2-Methoxyphenyl)-1,3,5-triazin-2-yl)amino]benzenemethanesulfonamide (Cpd B1);
3-[(4-(4-Fluoro-2-methoxyphenyl)-1,3,5-triazin-2-yl)amino]benzenemethanesulfonamide (Cpd B2);
3-[(4-(5-Fluoro-2-methoxyphenyl)-1,3,5-triazin-2-yl)amino]benzenemethanesulfonamide (Cpd B3);
3-[(4-(6-Fluoro-2-methoxyphenyl)-1,3,5-triazin-2-yl)amino]benzenemethanesulfonamide (Cpd B4);
3-[(4-(4-Chloro-2-methoxyphenyl)-1,3,5-triazin-2-yl)amino]benzenemethanesulfonamide (Cpd B6);
3-[(4-(5-Chloro-2-methoxyphenyl)-1,3,5-triazin-2-yl)amino]benzenemethanesulfonamide (Cpd B7);
3-[(4-(2-Ethoxyphenyl)-1,3,5-triazin-2-yl)amino]benzenemethanesulfonamide (Cpd B12);
3-[(4-(2-Benzyloxyphenyl)-1,3,5-triazin-2-yl)amino]benzenemethanesulfonamide (Cpd B13);
1-(3-{[4-(2-phenoxyphenyl)-1,3,5-triazin-2-yl]amino}phenyl)methanesulfonamide (Cpd B14);
3-[(4-(2-((4-Pyridinyl)methoxy)phenyl)-1,3,5-triazin-2-yl)amino]benzenemethanesulfonamide (Cpd B16);
3-[(4-(2-(4-(tert-Butoxycarbonylamino)butoxy)phenyl)-1,3,5-triazin-2-yl)amino]benzenemethanesulfonamide (Cpd B17);
3-[(4-(3-Methoxypyridin-4-yl)-1,3,5-triazin-2-yl)amino]benzenemethanesulfonamide (Cpd B18);
3-[(4-(6-Aminopyridin-3-yl)-1,3,5-triazin-2-yl)amino]benzenemethanesulfonamide (Cpd B23);
3-[(4-(2-Methoxyphenyl)-1,3,5-triazin-2-yl)amino]benzenesulfonamide (Cpd B24);
2-[3-((4-(2-Methoxyphenyl)-1,3,5-triazin-2-yl)amino)phenyl]ethanesulfonamide (Cpd C1);
N-[3-((4-(2-Methoxyphenyl)-1,3,5-triazin-2-yl)amino)phenyl]-methanesulfonamide (Cpd D1);
N-[3-((4-(2-Methoxyphenyl)-1,3,5-triazin-2-yl)amino)phenyl]-propanesulfonamide (Cpd L1);
tert-Butyl[4-((3-((4-(4-Fluoro-2-methoxyphenyl)-1,3,5-triazin-2-yl)amino)phenyl)methylsulfonamido)butyl]carbamate (Cpd Q1);
N-(4-Aminobutyl)-1-[3-((4-(4-fluoro-2-methoxyphenyl)-1,3,5-triazin-2-yl)amino)phenyl]methanesulfonamide (Cpd R1);
4-(2-Methoxyphenyl)-N-(3-(methylsulfonyl)phenyl)-1,3,5-triazin-2-amine (Cpd S1);
1-[3-({4-[4-Fluoro-2-(propan-2-yloxy)phenyl]-1,3,5-triazin-2-yl}amino)phenyl]methanesulfonamide (Cpd U2);
1-[3-({4-[2-(Cyclopropylmethoxy)-4-fluorophenyl]-1,3,5-triazin-2-yl}amino)phenyl]methanesulfonamide (Cpd U5);
1-(3-{[4-(4,5-Difluoro-2-methoxyphenyl)-1,3,5-triazin-2-yl]amino}phenyl)methanesulfonamide (Cpd U7);
3-[(4-(2-Methoxyphenyl)pyridin-2-yl)amino]benzenesulfonamide (Cpd 24);
4-(2-Methoxyphenyl)-N-(3-(methylsulfonyl)phenyl)pyridin-2-amine (Cpd 25);
[3-((4-(4-Fluoro-2-methoxyphenyl)pyridin-2-yl)amino)phenyl]methanesulfonamide (Cpd 26);
[3-((4-(2-Methoxyphenyl)pyridin-2-yl)amino)phenyl]methanesulfonamide (Cpd 27);
1-[3-((4-(4-Fluoro-2-methoxyphenyl)pyridin-2-yl)amino)phenyl]-N,N-dimethylmethanesulfonamide (Cpd 28);
2-[3-((4-(2-Methoxyphenyl)pyridin-2-yl)amino)phenyl]ethanesulfonamide (Cpd 29);
N-[3-((4-(4-Fluoro-2-methoxyphenyl)pyridin-2-yl)amino)phenyl]methanesulfonamide (Cpd 30);
N-[3-((4-(4-Fluoro-2-methoxyphenyl)pyridin-2-yl)amino)phenyl]acetamide (Cpd 31);
1-[3-((4-(4-Fluoro-2-methoxyphenyl)pyridin-2-yl)amino)phenyl]-N-propylmethanesulfonamide (Cpd 32);
(R)-Methyl 1-[4-((3-(Sulfamoylmethyl)phenyl)amino)-1,3,5-triazin-2-yl]piperidine-2-carboxylate (Cpd 33);
(R)-3-[(4-(2-(Methoxymethyl)pyrrolidin-1-yl)-1,3,5-triazin-2-yl)amino]benzenemethanesulfonamide (Cpd 34);
(R)-Methyl 1-[4-((3-(Sulfamoylmethyl)phenyl)amino)-1,3,5-triazin-2-yl]pyrrolidine-2-carboxylate) Cpd 35);
rac-3-[(4-(2-Phenylpyrrolidin-1-yl)-1,3,5-triazin-2-yl)amino]benzenemethanesulfonamide (Cpd 36);
(R)-3-[(4-(2-Phenylpyrrolidin-1-yl)-1,3,5-triazin-2-yl)amino]benzenemethanesulfonamide (Cpd 37);
3-[(4-(7,8-Dihydro-1,6-naphthyridin-6(5H)-yl)-1,3,5-triazin-2-yl)amino]benzenemethanesulfonamide (Cpd 38);
3-[(4-(3,4-Dihydroquinolin-[(2H)-yl)-1,3,5-triazin-2-yl)amino]benzenemethanesulfonamide (Cpd 39);
3-[(4-(6,7-Dihydro-3H-imidazo[4,5-c]pyridin-5(4H)-yl)-1,3,5-triazin-2-yl)amino]benzenemethanesulfonamide (Cpd 40);
3-[(4-(1H-Pyrrolo[3,4-c]pyridin-2(3H)-yl)-1,3,5-triazin-2-yl)amino]benzenemethanesulfonamide (Cpd 41);
3-[(4-(Pyrrolo[3,4-c]pyrazol-5(1H,4H,6H)-yl)-1,3,5-triazin-2-yl)amino]benzenemethanesulfonamide (Cpd 42);
3-[(4-(Indolin-1-yl)-1,3,5-triazin-2-yl)amino]benzenemethanesulfonamide (Cpd 43); and
(S)-3-[(4-(2-Methylpyrrolidin-1-yl)-1,3,5-triazin-2-yl)amino]benzenemethanesulfonamide (Cpd 44).

12. The method according to claim 2, wherein said inhibitor is selected from the group consisting of,
Piperidine-4-carboxylic acid [5-(5-tert-butyl-oxazol-2-ylmethylsulfanyl)-thiazol-2-yl]-amide (SNS-032);
2-(2-Chloro-phenyl)-5,7-dihydroxy-8-(3-hydroxy-1-methyl-piperidin-4-yl)-chromen-4-one, (flavopiridol);
N-(5-((6-(3-aminophenyl)pyrimidin-4-yl)amino)-2-methylphenyl)propane-1-sulfonamide (AX35427);
[4-amino-2-(1-methanesulfonylpiperidin-4-ylamino)pyrimidin-5-yl]-(2,3-difluoro-6-methoxyphenyl)methanone (R-547);
3-[[6-(2-methoxyphenyl)-4-pyrimidinyl]amino]-Benzenemethanesulfonamide (1073485-20-7P);
3-((6-(2-methoxyphenyl)pyrimidin-4-yl)amino)benzenesulfonamide (AX38679);
1,5,6,7-tetrahydro-2-(4-pyridinyl)-4H-pyrrolo[3,2-c]pyridin-4-one, (PHA767491);
5,6-dichloro-1-b-ribofuranosyl-benzimidazole (DRB);
6-Benzylamino-2[(R)-(1′-ethyl-2′-hydroxyethylamino)]-9-isopropylpurine (Roscovitine);
4-[[4-Amino-5-(2,6-difluorobenzoyl)thiazol-2-yl]amino]-N-((R)-2-dimethylamino-1-methylethyl)benzamide (AG-012986);
4H-1-Benzopyran-4-one, 2-(2-chlorophenyl)-5,7-dihydroxy-8-[(2R,3S)-2-(hydroxymethyl)-1-methyl-3-pyrrolidinyl]-, hydrochloride (1:1) (P276-00);
4-[3-Chloro-5-(4-methylpiperazin-1-yl)benzoylamino]-1H-pyrazole-3-carboxylic acid cyclohexylamide (ZK 304709);
4-(2,6-Dichlorobenzoylamino)-1H-pyrazole-3-carboxylic acid N-(piperidin-4-yl)amide (AT7519);
N-[2-(dimethylamino)ethyl]-2-fluoro-4-[[5-fluoro-4-[2-methyl-1-(1-methylethyl)-1H-imidazol-5-yl]-2-pyrimidinyl]amino] (Compound 7d);
[4-[[5-fluoro-4-[2-methyl-1-(1-methyl ethyl)-1H-imidazol-5-yl]-2-pyrimidinyl]amino]phenyl][(3S)-3-(methylamino)-1-pyrrolidinyl](AZD5597);
N-[1,4-dihydro-3-[4-[[4-(2-methoxyethyl)-1-piperazinyl]methyl]phenyl]-4-oxoindeno[1,2-c]pyrazol-5-yl]-N′-4-morpholinyl-, hydrochloride (1:2) (RGB-286638); and
4-(2,6-dichlorobenzamido)-N-(1-(methylsulfonyl)piperidin-4-yl)-1H-pyrazole-3-carboxamide (LCQ195AT9311);

13. The method according to claim 3, wherein said NUT midline carcinoma (NMC) is characterized by the presence of at least one rearrangement in the NUT gene in a tumor or cancer cell in said NUT midline carcinoma (NMC).

14. The method according to claim 13, wherein said rearrangement in the NUT gene is a t15;19 translocation as reflected in formation of a Brd4/NUT fusion gene.

15. The method according to claim 13, wherein said rearrangement in the NUT gene is a formation of a NUT variant fusion gene.

16. The method according to claim 13, wherein said NUT variant fusion gene is a Brd3/NUT fusion gene.

17. The method according to claim 13, wherein said rearrangement in the NUT gene is reflected in expression of the formed NUT fusion gene and whereby the expression level of the formed NUT fusion gene is detected.

18. The method according to claim 17, wherein the expression level is detected by an immunohistochemical method, real-time PCR, and/or Northern Blot.

19. The method according to claim 13, wherein said rearrangement in the NUT gene is detected by an in situ hybridization method.

20. The method according to claim 19, wherein the in situ hybridization method is selected from the group consisting of fluorescent in situ hybridization (FISH), chromogenic in situ hybridization (CISH) and silver in situ hybridization (SISH).

21. The method of claim 2, wherein the subject is a human.

22. A pharmaceutical composition comprising a CDK9 inhibitor according to claim 5, and a pharmaceutically acceptable carrier.


SEQUENCE LISTING (12 sequences)

SEQ ID NO: 1 - DNA, 3795 bp, Homo sapiens
gagttccgtattctagttctgtgtgatctgatctttaccttcccttccttggatccctgtgcacctactggagccaggttactctgggtcctggacctgactgcctcattctggaggcttccagacagccacagttagtgcccaaacctgagaggatggcttcagatggagcatctgcattgccgggaccggatatgagcatgaaacctagtgccgccctgtctccatcccctgcacttccctttctcccaccaacttctgacccaccagaccacccacccagggagccacctccacagcccatcatgccttcagtattctctccagacaaccctctgatgctctctgctttccccagctcactgttggtgacaggggacgggggcccttgcctcagtggggctggggctggcaaggtcattgtcaaagtcaagacagaaggggggtcagctgagccctctcaaactcagaactttatccttactcagactgccctcaattcgactgccccgggcactccctgtggaggccttgagggtcctgcacctccatttgtgacagcatctaatgtgaagaccattctgccctctaaggctgttggtgtcagccaggagggtcctccaggccttccgcctcagcctccaccaccagttgctcaactggtccccattgtgcccctggaaaaagcttggccagggccacatgggacaaccggggaaggaggtcctgtggccactctatccaagccttccctaggtgaccgctccaaaatttccaaggacgtttatgagaacttccgtcagtggcagcgttacaaagccttggcccggaggcacctatcccagagtcctgacacagaagctctttcctgttttcttatcccagtgcttcgttccctggcccggctgaagcccactatgaccctggaggagggactgccattggctgtgcaggagtgggagcacaccagcaactttgaccggatgatcttttatgagatggcagaaaggttcatggagtttgaggctgaggagatgcagattcagaacacacagctgatgaatgggtctcagggcctgtctcctgcaacccctttgaaacttgatcctctagggcccctggcctctgaggtttgccagcagccagtgtacattccgaagaaggcagcctccaagacacgggccccccgccggcgtcagcgtaaagcccagagacctcctgctcctgaggcacccaaggagatcccaccagaagctgtgaaggagtatgttgacatcatggaatggctggtggggactcacttggccactggggagtcagatggaaaacaagaggaagaagggcagcagcaggaggaggaagggatgtatccagatccaggtctcctgagctacatcaatgagctgtgttctcagaaggtctttgtctccaaggtggaggctgtcattcaccctcaatttctggcagatctgctgtccccagaaaaacagagagatcccttggccttaattgaggagctagagcaagaagaaggactcactcttgcccagctggtccagaagcgactcatggccttggaagaggaggaagatgcagaggcgcctccaagtttcagtggcgctcagttggactcaagtccttctggttctgttgaggatgaagatggggatgggcggcttcggccctcacctgggcttcagggggctgggggcgccgcttgccttggaaaggtttcttcttcaggaaaacgggcaagagaagtgcatggtgggcaggagcaagccctagatagccccagagggatgcacagggatgggaacactctgccatcccccagcagctgggacctgcagccagaacttgcagctccacagggaactccgggacccttgggtgtggagaggagagggtctgggaaggttataaaccaggtatctctacatcaggatggccatctaggaggcgctgggcctcctgggcactgcctggtggctgataggacttcagaggctctgcccctttgttggcagggaggcttccagcctgagagcactcccagtttggatgctggacttgcagagctggctcctctgcaaggacaagggttagaaaagcaagtcctgggattgcagaaaggacaacaaacagggggtcgtggagtgcttcctcaagggaaggagcctttagcagtgccctgggaaggctcttcaggagccatgtggggagatgacagaggtacccccatggctcagagttatgatcagaatccttcccctagagcagctggggagagggacgatgtctgtctcagcccaggagtttggctgagcagtgagatggatgctgtaggcttggagctgcctgtacaaatagaggaggtcatagagagcttccaagttgagaagtgtgtaactgagtatcaggaaggctgccagggactgggctccaggggcaacatttccctgggtcctggagaaaccctagtacctggggatacggagagcagtgtgattccctgtggaggcacagttgcggcagctgccctagaaaagagaaactattgcagcttgccaggacctttgagggccaacagcccacccttgaggtccaaagaaaatcaagaacagagctgtgaaaccgtagggcatcccagtgatctgtgggcagaaggttgcttcccattgctagaaagtggtgattccacactggggtcttccaaagaaacccttccacccacatgccaaggcaatctccttatcatggggactgaggatgcctcctccttgcctgaagccagtcaagaggcagggagcagaggcaattccttttctcctctgttggaaaccatagaacctgtcaacatactagatgttaaagatgactgtggcctccaactaagggtcagcgaggacacctgcccactgaatgttcattcttatgacccccaaggagaaggcagggtggatcctgatctgtccaagcctaaaaaccttgctcctttacaagagagtcaggagtcttacacaactgggactcccaaagcaacatcttctcaccagggccttggaagcactttgcctagaaggggaaccaggaatgccatagttccgagagaaacttctgttagtaaaacacacaggtcagcagacagggccaaaggaaaggagaaaaagaaaaaggaagcagaggaagaggatgaggaactctccaactttgcttacctcttggcctctaaacttagcctctcaccaagggagcatcccctcagtcctcaccatgcctcaggaggtcagggcagccagagagcatcccacctgctccctgctggagcaaaaggccccagcaaacttccatatcctgttgccaagtctgggaagcgagctctagctggaggtccagcccctactgaaaagacaccccactcaggagctcaacttggggtccccagggagaaacccctagctctgggagtagttcgaccctcacagcctcgtaaaaggcggtgtgacagttttgtcacgggcagaaggaagaaacgacgtcgtagccagtagggagcagcgggaccatctgaccccacttgccagtccctaaaggtgggtgccccagagtagattccacccctgctgcccaccaatggagaatcccaatgttgaatctcatcccaatgttgttttgttgttctgcaaaagtggcaagcatggagagagaggtcagactggctaggctgcagggggaattacctttggaaggagctatatagaaaaaaaatgaataaagtgttttgttggaaaa

SEQ ID NO: 2 - protein, 1132 aa, Homo sapiens
MASDGASALPGPDMSMKPSAALSPSPALPFLPPTSDPPDHPPREPPPQPIMPSVFSPDNPLMLSAFPSSLLVTGDGGPCLSGAGAGKVIVKVKTEGGSAEPSQTQNFILTQTALNSTAPGTPCGGLEGPAPPFVTASNVKTILPSKAVGVSQEGPPGLPPQPPPPVAQLVPIVPLEKAWPGPHGTTGEGGPVATLSKPSLGDRSKISKDVYENFRQWQRYKALARRHLSQSPDTEALSCFLIPVLRSLARLKPTMTLEEGLPLAVQEWEHTSNFDRMIFYEMAERFMEFEAEEMQIQNTQLMNGSQGLSPATPLKLDPLGPLASEVCQQPVYIPKKAASKTRAPRRRQRKAQRPPAPEAPKEIPPEAVKEYVDIMEWLVGTHLATGESDGKQEEEGQQQEEEGMYPDPGLLSYINELCSQKVFVSKVEAVIHPQFLADLLSPEKQRDPLALIEELEQEEGLTLAQLVQKRLMALEEEEDAEAPPSFSGAQLDSSPSGSVEDEDGDGRLRPSPGLQGAGGAACLGKVSSSGKRAREVHGGQEQALDSPRGMHRDGNTLPSPSSWDLQPELAAPQGTPGPLGVERRGSGKVINQVSLHQDGHLGGAGPPGHCLVADRTSEALPLCWQGGFQPESTPSLDAGLAELAPLQGQGLEKQVLGLQKGQQTGGRGVLPQGKEPLAVPWEGSSGAMWGDDRGTPMAQSYDQNPSPRAAGERDDVCLSPGVWLSSEMDAVGLELPVQIEEVIESFQVEKCVTEYQEGCQGLGSRGNISLGPGETLVPGDTESSVIPCGGTVAAAALEKRNYCSLPGPLRANSPPLRSKENQEQSCETVGHPSDLWAEGCFPLLESGDSTLGSSKETLPPTCQGNLLIMGTEDASSLPEASQEAGSRGNSFSPLLETIEPVNILDVKDDCGLQLRVSEDTCPLNVHSYDPQGEGRVDPDLSKPKNLAPLQESQESYTTGTPKATSSHQGLGSTLPRRGTRNAIVPRETSVSKTHRSADRAKGKEKKKKEAEEEDEELSNFAYLLASKLSLSPREHPLSPHHASGGQGSQRASHLLPAGAKGPSKLPYPVAKSGKRALAGGPAPTEKTPHSGAQLGVPREKPLALGVVRPSQPRKRRCDSFVTGRRKKRRRSQ

SEQ ID NO: 3 - DNA, 5198 bp, Homo sapiens
attctttggaatactactgctagaagtctgacttaagacccagcttatgggccacatggcacccagctgcttctgcagagaaggcaggccactgatgggtacagcaaagtgtggtgctgctggccaagccaaagacccgtgtaggatgactgggcctctgccccttgtgggtgttgccactgtgcttgagtgcctggtgaagaatgtgatgggatcactagcatgtctgcggagagcggccctgggacgagattgagaaatctgccagtaatgggggatggactagaaacttcccaaatgtctacaacacaggcccaggcccaaccccagccagccaacgcagccagcaccaaccccccgcccccagagacctccaaccctaacaagcccaagaggcagaccaaccaactgcaatacctgctcagagtggtgctcaagacactatggaaacaccagtttgcatggcctttccagcagcctgtggatgccgtcaagctgaacctccctgattactataagatcattaaaacgcctatggatatgggaacaataaagaagcgcttggaaaacaactattactggaatgctcaggaatgtatccaggacttcaacactatgtttacaaattgttacatctacaacaagcctggagatgacatagtcttaatggcagaagctctggaaaagctcttcttgcaaaaaataaatgagctacccacagaagaaaccgagatcatgatagtccaggcaaaaggaagaggacgtgggaggaaagaaacagggacagcaaaacctggcgtttccacggtaccaaacacaactcaagcatcgactcctccgcagacccagacccctcagccgaatcctcctcctgtgcaggccacgcctcaccccttccctgccgtcaccccggacctcatcgtccagacccctgtcatgacagtggtgcctccccagccactgcagacgcccccgccagtgcccccccagccacaacccccacccgctccagctccccagcccgtacagagccacccacccatcatcgcggccaccccacagcctgtgaagacaaagaagggagtgaagaggaaagcagacaccaccacccccaccaccattgaccccattcacgagccaccctcgctgcccccggagcccaagaccaccaagctgggccagcggcgggagagcagccggcctgtgaaacctccaaagaaggacgtgcccgactctcagcagcacccagcaccagagaagagcagcaaggtctcggagcagctcaagtgctgcagcggcatcctcaaggagatgtttgccaagaagcacgccgcctacgcctggcccttctacaagcctgtggacgtggaggcactgggcctacacgactactgtgacatcatcaagcaccccatggacatgagcacaatcaagtctaaactggaggcccgtgagtaccgtgatgctcaggagtttggtgctgacgtccgattgatgttctccaactgctataagtacaaccctcctgaccatgaggtggtggccatggcccgcaagctccaggatgtgttcgaaatgcgctttgccaagatgccggacgagcctgaggagccagtggtggccgtgtcctccccggcagtgccccctcccaccaaggttgtggccccgccctcatccagcgacagcagcagcgatagctcctcggacagtgacagttcgactgatgactctgaggaggagcgagcccagcggctggctgagctccaggagcagctcaaagccgtgcacgagcagcttgcagccctctctcagccccagcagaacaaaccaaagaaaaaggagaaagacaagaaggaaaagaaaaaagaaaagcacaaaaggaaagaggaagtggaagagaataaaaaaagcaaagccaaggaacctcctcctaaaaagacgaagaaaaataatagcagcaacagcaatgtgagcaagaaggagccagcgcccatgaagagcaagccccctcccacgtatgagtcggaggaagaggacaagtgcaagcctatgtcctatgaggagaagcggcagctcagcttggacatcaacaagctccccggcgagaagctgggccgcgtggtgcacatcatccagtcacgggagccctccctgaagaattccaaccccgacgagattgaaatcgactttgagaccctgaagccgtccacactgcgtgagctggagcgctatgtcacctcctgtttgcggaagaaaaggaaacctcaagctgagaaagttgatgtgattgccggctcctccaagatgaagggcttctcgtcctcagagtcggagagctccagtgagtccagctcctctgacagcgaagactccgaaacagagatggctccgaagtcaaaaaagaaggggcaccccgggagggagcagaagaagcaccatcatcaccaccatcagcagatgcagcaggccccggctcctgtgccccagcagccgcccccgcctccccagcagcccccaccgcctccacctccgcagcagcaacagcagccgccacccccgcctcccccaccctccatgccgcagcaggcagccccggcgatgaagtcctcgcccccacccttcattgccacccaggtgcccgtcctggagccccagctcccaggcagcgtctttgaccccatcggccacttcacccagcccatcctgcacctgccgcagcctgagctgccccctcacctgccccagccgcctgagcacagcactccaccccatctcaaccagcacgcagtggtctctcctccagctttgcacaacgcactaccccagcagccatcacggcccagcaaccgagccgctgccctgcctcccaagcccgcccggcccccagccgtgtcaccagccttgacccaaacacccctgctcccacagccccccatggcccaacccccccaagtgctgctggaggatgaagagccacctgccccacccctcacctccatgcagatgcagctgtacctgcagcagctgcagaaggtgcagccccctacgccgctactcccttccgtgaaggtgcagtcccagcccccaccccccctgccgcccccaccccacccctctgtgcagcagcagctgcagcagcagccgccaccacccccaccaccccagccccagcctccaccccagcagcagcatcagccccctccacggcccgtgcacttgcagcccatgcagttttccacccacatccaacagcccccgccaccccagggccagcagcccccccatccgcccccaggccagcagccacccccgccgcagcctgccaagcctcagcaagtcatccagcaccaccattcaccccggcaccacaagtcggacccctactcaaccggtcacctccgcgaagccccctccccgcttatgatacattccccccagatgtcacagttccagagcctgacccaccagtctccaccccagcaaaacgtccagcctaagaaacaggagctgcgtgctgcctccgtggtccagccccagcccctcgtggtggtgaaggaggagaagatccactcacccatcatccgcagcgagcccttcagcccctcgctgcggccggagccccccaagcacccggagagcatcaaggcccccgtccacctgccccagcggccggaaatgaagcctgtggatgtcgggaggcctgtgatccggcccccagagcagaacgcaccgccaccaggggcccctgacaaggacaaacagaaacaggagccgaagactccagttgcgcccaaaaaggacctgaaaatcaagaacatgggctcctgggccagcctagtgcagaagcatccgaccaccccctcctccacagccaagtcatccagcgacagcttcgagcagttccgccgcgccgctcgggagaaagaggagcgtgagaaggccctgaaggctcaggccgagcacgctgagaaggagaaggagcggctgcggcaggagcgcatgaggagccgagaggacgaggatgcgctggagcaggcccggcgggcccatgaggaggcacgtcggcgccaggagcagcagcagcagcagcgccaggagcaacagcagcagcagcaacagcaagcagctgcggtggctgccgccgccaccccacaggcccagagctcccagccccagtccatgctggaccagcagagggagttggcccggaagcgggagcaggagcgaagacgccgggaagccatggcagctaccattgacatgaatttccagagtgatctattgtcaatatttgaagaaaatcttttctgagcgcacctaggtggcttctgactttgattttctggcaaaacattgactttccatagtgttaggggcggtggtggaggtgggatcagcggccaggggatgcctcagggcctggccctcctgcatgctatgcccggggcaggcctgacgggcagctgaggattgcagagcctgtctgccttacggccagtcggacagacgtcccgccacccaccacccctcacaggacgtccgctcagcacacgccttgttacgagcaagtgccggctggacccaagccctgcatccccacatgcggggcagaggcccttctctccgccaaatgtctacacagtatacacaggacatcgttgctgccgccgtgactggttttctgtccccaagaacgtgacgttcgtgatgtcctgcccgccgggagtctttccccacaccccagccatcgccgcccgctcccaggaggccagggcaggcctgcgtgggctggaggcgggcgaggccggcccaccccctcgctggcactgactttgccttgaacagaccccccgaccctcccccacaagcctttaattgagagccgctctctgtaagtgtttgcttgtgcaaaagggaatagtgccgtggaggtgtgtgtgtccatggcatccggagcgaggcgactgtcctgcgtgggtagccctcggccggggagtgaggccaccaaccaaagtcagttccttcccacctgtgtttctgtttcgtttttttttttcttttttttctatatatattttttgttgaattctattttatttttaattctctcttctcctccagacacaatggcactgcttatctccgaaatggtgtgatcgtctcctcattgagcagcggctgccaccgcgctgtgggta

SEQ ID NO: 4 - protein, 1362 aa, Homo sapiens
MSAESGPGTRLRNLPVMGDGLETSQMSTTQAQAQPQPANAASTNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEETEIMIVQAKGRGRGRKETGTAKPGVSTVPNTTQASTPPQTQTPQPNPPPVQATPHPFPAVTPDLIVQTPVMTVVPPQPLQTPPPVPPQPQPPPAPAPQPVQSHPPIIAATPQPVKTKKGVKRKADTTTPTTIDPIHEPPSLPPEPKTTKLGQRRESSRPVKPPKKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDEPEEPVVAVSSPAVPPPTKVVAPPSSSDSSSDSSSDSDSSTDDSEEERAQRLAELQEQLKAVHEQLAALSQPQQNKPKKKEKDKKEKKKEKHKRKEEVEENKKSKAKEPPPKKTKKNNSSNSNVSKKEPAPMKSKPPPTYESEEEDKCKPMSYEEKRQLSLDINKLPGEKLGRVVHIIQSREPSLKNSNPDEIEIDFETLKPSTLRELERYVTSCLRKKRKPQAEKVDVIAGSSKMKGFSSSESESSSESSSSDSEDSETEMAPKSKKKGHPGREQKKHHHHHHQQMQQAPAPVPQQPPPPPQQPPPPPPPQQQQQPPPPPPPPSMPQQAAPAMKSSPPPFIATQVPVLEPQLPGSVFDPIGHFTQPILHLPQPELPPHLPQPPEHSTPPHLNQHAVVSPPALHNALPQQPSRPSNRAAALPPKPARPPAVSPALTQTPLLPQPPMAQPPQVLLEDEEPPAPPLTSMQMQLYLQQLQKVQPPTPLLPSVKVQSQPPPPLPPPPHPSVQQQLQQQPPPPPPPQPQPPPQQQHQPPPRPVHLQPMQFSTHIQQPPPPQGQQPPHPPPGQQPPPPQPAKPQQVIQHHHSPRHHKSDPYSTGHLREAPSPLMIHSPQMSQFQSLTHQSPPQQNVQPKKQELRAASVVQPQPLVVVKEEKIHSPIIRSEPFSPSLRPEPPKHPESIKAPVHLPQRPEMKPVDVGRPVIRPPEQNAPPPGAPDKDKQKQEPKTPVAPKKDLKIKNMGSWASLVQKHPTTPSSTAKSSSDSFEQFRRAAREKEEREKALKAQAEHAEKEKERLRQERMRSREDEDALEQARRAHEEARRRQEQQQQQRQEQQQQQQQQAAAVAAAATPQAQSSQPQSMLDQQRELARKREQERRRREAMAATIDMNFQSDLLSIFEENLF

SEQ ID NO: 5 - DNA, 5673 bp, Homo sapiens
tgccggggccggcgagccaaagaggagccggccgcgcgggccgggaggggacggccgccggagccgcgaggccaactgtcgcctggttgggcccggaaatgggacgtcgcgctttctcagggagcgtagaagcagccagggcctctccaagccgctgctgtgacagaaagtgagtgagctgccggaggatgtccaccgccacgacagtcgcccccgcggggatcccggcgaccccgggccctgtgaacccaccccccccggaggtctccaaccccagcaagcccggccgcaagaccaaccagctgcagtacatgcagaatgtggtggtgaagacgctctggaaacaccagttcgcctggcccttctaccagcccgtggacgcaatcaaattgaacctgccggattatcataaaataattaaaaacccaatggatatggggactattaagaagagactagaaaataattattattggagtgcaagcgaatgtatgcaggacttcaacaccatgtttacaaattgttacatttataacaagcccacagatgacatagtgctaatggcccaagctttagagaaaatttttctacaaaaagtggcccagatgccccaagaggaagttgaattattaccccctgctccaaagggcaaaggtcggaagccggctgcgggagcccagagcgcaggtacacagcaagtggcggccgtgtcctctgtctccccagcgaccccctttcagagcgtgccccccaccgtctcccagacgcccgtcatcgctgccacccctgtaccaaccatcactgcaaacgtcacgtcggtcccagtccccccagctgccgccccacctcctcctgccacacccatcgtccccgtggtccctcctacgccgcctgtcgtcaagaaaaagggcgtgaagcggaaagcagacacaaccactcccacgacgtcggccatcactgccagccggagtgagtcgcccccgccgttgtcagaccccaagcaggccaaagtggtggcccggcgggagagtggtggccgccccatcaagcctcccaagaaggacctggaggacggcgaggtgccccagcacgcaggcaagaagggcaagctgtcggagcacctacgctactgcgacagcatcctcagggagatgctatccaagaagcacgcggcctacgcctggcccttctacaagccagtggatgccgaggccctggagctgcacgactaccacgacatcatcaagcacccgatggacctcagcaccgtgaaaaggaagatggatggccgagagtacccagacgcacagggctttgctgctgatgtccggctgatgttctcgaattgctacaaatacaatcccccagaccacgaggttgtggccatggcccggaagctccaggacgtgtttgagatgaggtttgccaagatgccagatgagcccgtggaggcaccggcgctgcctgcccccgcggcccccatggtgagcaagggcgctgagagcagccgtagcagtgaggagagctcttcggactcaggcagctcggactcggaggaggagcgggccaccaggctggcggagctgcaggagcagctgaaggccgtgcacgagcagctggccgccctgtctcaggccccagtaaacaaaccaaagaagaagaaggagaagaaggagaaggagaagaagaagaaggacaaggagaaggagaaggagaagcacaaagtgaaggccgaggaagagaagaaggccaaggtggctccgcctgccaagcaggctcagcagaagaaggctcctgccaagaaggccaacagcacgaccacggccggcagacagctgaagaaaggcggcaagcaggcatctgcctcctacgactcagaggaagaggaggagggcctgcccatgagctacgatgaaaagcgccagcttagcctggacatcaaccggctgcccggggagaagctgggccgggtagtgcacatcatccaatctcgggagccctcgctcagggactccaaccccgacgagatagaaattgactttgagactctgaaacccaccactttgcgggaactggagagatatgtcaagtcttgtttacagaaaaagcaaaggaaaccgttctcagcaagcgggaagaaacaggcagccaagtcgaaagaggagctagctcaggaaaagaagaaggagctggaaaagcgtctgcaggatgtcagcgggcagctgagcagcagcaagaagcccgcccggaaagagaagcccggctcagcaccctcagggggcccgtccaggctcagcagcagcagctcctccgagtctgggagcagcagctccagcgggtccagctctgacagcagtgactcagaatgaactggcttcggacagaacaggacagatggatgtcgcacacgccgagactctgccgtacccctctgtggttcatattactacttctgttccatggtgtgcaggtctgcttcttaattcagtgttatgatatcttccagtttttgctttcataggtcagagatctatcttgtgtgtgcgttagacttgatgagaaggtgtgaactctgcagaaagtctcttcttcatcactgaattcagtcacttggagatgacaacttcaaatgctaacccgatgaccccagaaaaccgtgtgagattcgtaccgaagaaccttgtggaatccctttgcttaggcccaacctggtcgatagctcgagaaagaattttttccaaggaaatgtctcggatatgggtactgtatttgaaagctgttagctttgtcaacacgcattgtccttgtcatttgggccccgagctctgaccctcgtgtctgacgcggccacctctttctggaggggctgaggacagatgtgcctgcttgtggagaccaggctgggcctaagcgaagggtcatcgcagccccagcccggagcgtggagcccttggggggtggtcgggtgggatgtgcgttctccgctcgtggtgatgtcaggagctcctcggagggaacagagcggctgtgtatgcagcctgcaggtttccatacactgaagcttttacctcaactttaaaaaaaaaaaaagaagaaaagatttaaaaaaaaaaaaaaaggagactttttttgtaaggttcggcaattttctacggaagtccagcccgctgtgagtccccgcctggctagcgctgcccctgctgctgctgccgcgccacccttgggaaccacttcccgagcttatgtgtatataaatagttatttattaacatcattggtcatttttaaaaaaaagaaaataagaaaaaaccgcagaagaaatgcattcacacagtcgcagagatgcaggccttgccagtggtgtgccgggcgcgggtcctgtctggcggcggcctgtcgtctccaggtgctaactcctgccaccgcgcggtgctcacccacgtctgttcgcgcgctcgcccctgggtttgttgggttttttggtttttttctttgtggtttttttttttttttttttttgtatgaaacttggaggcttacaggtatagacagctttcagctacagcacattctaattttttattttgtttagttcttttgtattcacttctggtctctttaagactgttttaaaagaaatcaatttagggaaccccagttatataatataaactttgtaatctgagagaaaaaatgtatagtaaatctaagtcttgatttttaactttctattgtaaaaaataataatatacagagtttaatagaaggtgatgttttggttttgttttcccagaggctgccatatggtctttgagtacggggatgtcccaaactggcccaccaatgagcatggcggctccggccaggaatgccagagttagcctcccaggcttgcgggtggacatgcctgctccctgccagcctccagtggcctggccaggccctcccgagcctgtctgccctccccaggggtggaggagtctctgggccccaggaggattccctcccggagactcgcacggtgctccctgctcacgcgttgtcacagttagtccggaaatgactgaaaccaggcattctcccggacctcagcgtgggggagcctccaggcagacgctgggtatggagctgtggtgtggtctgtcctgtatggtggccagtgctttctgccagcatttctggatggatatagggactatcattagtatcctaatacacggtgattttaaaacaaccataaaattgattcagagtccactgacccttacagatgtaggtatacccttactggagagggaactctgatgaggagatgctggtaaattatcattttttaaattgctggtgagtctgacacttggtgagttttcagccagtttgttaaacttttaattaagttttgtttataataaaaatataaatggatttgaaagtttccattttttaaagttaccctcgttttcaaaggtattttctaaacagatctttaatggactatttaaaccgaatttaaggaattcacacacgacagttgacaggtcttcacgcaggctggttggtaacgtgctgccagcacagggctgggtgatacgtacaccctaagccgggggtgcctggggctggggggcgctccttgcaatgcccctccagccacagggcagtgaggtgctgcctgtgtgagccgtcgggggagcggccggctgtgggggcagcgcagcaggagcatcgtggggcctttccttctcggctggttctctgtgacggtggcgtcggctcgcctctgctcctttcatctagaaagaagccactgaccctgacagcccacggcgggtacactgagcagctgcattggtgctgtcacttttttaaggctttctgtccagacttcaacactggtttcttttcagagtttcgaaggattaatgacttcctcagcgcccttgctggcgggctgagggtgacagtcacgtccgtttcttctgtattagaaggctgcggtgattcaattagattgtcccactgctgagacctgtagggcagcttctaacatgcttttttcaaggggagaggagtagtgacaagtcgtgtgtcggaattggatttgagaacactctgaatgacccctggaggccgagggggcaggcttcgggcgtgaactgaactccagacccctctttgtgttgggcagtgtcatcttgcttacaaactgtaagacacatttttttgtgtgtttgtttttgttgttgttcttttgcagcactcacgcctctgacagtcttttgggaaagagtaacacccacatacagaatttgtcacatccagagtagcactgttccttaatactggcataatgcttccaggaagtttttcttttttatatttaaaatgttacttttctgtatgatgtgcatgcaagtttaccgtaacttttcttaaactttttagtgccgtttctagtatattcctgtaaatgtcagttactgaaaatgagtccaatgtaagtagtttagcttgtttattgcaatgctggcctcaacacaacagaataaaaatggtagaaagtactctttgatgtttctggtaatcatggacccttctcctggggcatttgttttgttttcataataaaaagcaaaaaaaaaaaaaaaaaaaaaaaaaaaaaaaaaaaaaaaa

SEQ ID NO: 6 - protein, 726 aa, Homo sapiens
MSTATTVAPAGIPATPGPVNPPPPEVSNPSKPGRKTNQLQYMQNVVVKTLWKHQFAWPFYQPVDAIKLNLPDYHKIIKNPMDMGTIKKRLENNYYWSASECMQDFNTMFTNCYIYNKPTDDIVLMAQALEKIFLQKVAQMPQEEVELLPPAPKGKGRKPAAGAQSAGTQQVAAVSSVSPATPFQSVPPTVSQTPVIAATPVPTITANVTSVPVPPAAAPPPPATPIVPVVPPTPPVVKKKGVKRKADTTTPTTSAITASRSESPPPLSDPKQAKVVARRESGGRPIKPPKKDLEDGEVPQHAGKKGKLSEHLRYCDSILREMLSKKHAAYAWPFYKPVDAEALELHDYHDIIKHPMDLSTVKRKMDGREYPDAQGFAADVRLMFSNCYKYNPPDHEVVAMARKLQDVFEMRFAKMPDEPVEAPALPAPAAPMVSKGAESSRSSEESSSDSGSSDSEEERATRLAELQEQLKAVHEQLAALSQAPVNKPKKKKEKKEKEKKKKDKEKEKEKHKVKAEEEKKAKVAPPAKQAQQKKAPAKKANSTTTAGRQLKKGGKQASASYDSEEEEEGLPMSYDEKRQLSLDINRLPGEKLGRVVHIIQSREPSLRDSNPDEIEIDFETLKPTTLRELERYVKSCLQKKQRKPFSASGKKQAAKSKEELAQEKKKELEKRLQDVSGQLSSSKKPARKEKPGSAPSGGPSRLSSSSSSESGSSSSSGSSSDSSDSE

SEQ ID NO: 7 - DNA, 2472 bp, Homo sapiens
aagtggccgtggaggcggaagtggcgcggccgcggaggggcctggagtgcggcggcggcgggacccggagcaggagcggcggcagcagcgactgggggcggcggcggcgcgttggaggcggccatggcaaagcagtacgactcggtggagtgccctttttgtgatgaagtttccaaatacgagaagctcgccaagatcggccaaggcaccttcggggaggtgttcaaggccaggcaccgcaagaccggccagaaggtggctctgaagaaggtgctgatggaaaacgagaaggaggggttccccattacagccttgcgggagatcaagatccttcagcttctaaaacacgagaatgtggtcaacttgattgagatttgtcgaaccaaagcttccccctataaccgctgcaagggtagtatatacctggtgttcgacttctgcgagcatgaccttgctgggctgttgagcaatgttttggtcaagttcacgctgtctgagatcaagagggtgatgcagatgctgcttaacggcctctactacatccacagaaacaagatcctgcatagggacatgaaggctgctaatgtgcttatcactcgtgatggggtcctgaagctggcagactttgggctggcccgggccttcagcctggccaagaacagccagcccaaccgctacaccaaccgtgtggtgacactctggtaccggcccccggagctgttgctcggggagcgggactacggcccccccattgacctgtggggtgctgggtgcatcatggcagagatgtggacccgcagccccatcatgcagggcaacacggagcagcaccaactcgccctcatcagtcagctctgcggctccatcacccctgaggtgtggccaaacgtggacaactatgagctgtacgaaaagctggagctggtcaagggccagaagcggaaggtgaaggacaggctgaaggcctatgtgcgtgacccatacgcactggacctcatcgacaagctgctggtgctggaccctgcccagcgcatcgacagcgatgacgccctcaaccacgacttcttctggtccgaccccatgccctccgacctcaagggcatgctctccacccacctgacgtccatgttcgagtacttggcaccaccgcgccggaagggcagccagatcacccagcagtccaccaaccagagtcgcaatcccgccaccaccaaccagacggagtttgagcgcgtcttctgagggccggcgcttgccactagggctcttgtgttttttttcttctgctatgtgacttgcatcgtggagacagggcatttgagtttatatctctcatgcatattttatttaatccccaccctgggctctgggagcagcccgctgagtggactggagtggagcattggctgagagaccaggagggcactggagctgtcttgtccttgctggttttctggatggttcccagagggtttccatggggtaggaggatgggctcgcccaccagtgactttttctaagagctcccggcgtggtggaagaggggacaggtccctcacccacccacaatcctattctcgggctgagaaccctgcgtggggacagggctcgcctcaggaatgggctgtttttggcctaaccctcagaaacactggggctggcacaaactcttggtttcttcaacaggagaattttactgtgtttcttttggttccattgtttggagacattcctgggcacagtttggtccgttagaattaaaagttgaattttttttttttttaaatttttttttttcctccaggacttgtgtgttttgttctgcgcacacaccgccaactgttcccccacagtcagcagcaggttgggcctgaccattgggacttgattgtcaagtcactggaggtcttgacttttttatctcagttcatgttctcttccataattggaaaggacctttgtctgtttttcctcttgggtgccttccagaacgcatctcatgtccctggtgagggaattggtgagggcctgctgtgagctgctgtggctgcgatggtcacccagctgggcaaatcactggagtgacaatttgacctgtcacctgagaaggatggtccctcagactgctgggtagagggcctggggcaggctggagagagaaagtgggcagagggtgaagggatcacaggggtcttggaaggtggcagtagtttggacgggggtggggagtatgtgggagaaaaaaacagactgaaggtagaatccttgggaacctttgaggagcggcacattctggcaggcacgttttctgtgagcctgaagttaggaagagacattctggaggtcaatttcctacatcctcttacaggcggagaccttgaagtggggccaggaaggaaggttggcaaaacctttgaccagaactgtccttcatttacagaaactgacccagaccacaacacaaaaggccag

SEQ ID NO: 8 - protein, 372 aa, Homo sapiens
MAKQYDSVECPFCDEVSKYEKLAKIGQGTFGEVFKARHRKTGQKVALKKVLMENEKEGFPITALREIKILQLLKHENVVNLIEICRTKASPYNRCKGSIYLVFDFCEHDLAGLLSNVLVKFTLSEIKRVMQMLLNGLYYIHRNKILHRDMKAANVLITRDGVLKLADFGLARAFSLAKNSQPNRYTNRVVTLWYRPPELLLGERDYGPPIDLWGAGCIMAEMWTRSPIMQGNTEQHQLALISQLCGSITPEVWPNVDNYELYEKLELVKGQKRKVKDRLKAYVRDPYALDLIDKLLVLDPAQRIDSDDALNHDFFWSDPMPSDLKGMLSTHLTSMFEYLAPPRRKGSQITQQSTNQSRNPATTNQTEFERVF

SEQ ID NO: 9 - DNA, 2568 bp, Homo sapiens
gggaagatacacggtttttaagaaagaacttctaaccccactccaccgcccaacggtcacgtgacgcgcctgcgtcctcgcctcaaggttttttctggtcgcatattggctgttaactcggctacggggtgtagcgaggtgcattccggaagtaggtcctttgacttttgcttcctctacggaggcaagtaacaacccggcggtcgacgcttagcggaagttcgtcaagtcgcagttgcccccgcacagcggatgtgggtcgcctcttaggtgacgctgggaagtgcctgcaaccttcgccgctgccttctggttgaagcactatggagggagagaggaagaacaacaacaaacggtggtatttcactcgagaacagctggaaaatagcccatcccgtcgttttggcgtggacccagataaagaactttcttatcgccagcaggcggccaatctgcttcaggacatggggcagcgtcttaacgtctcacaattgactatcaacactgctatagtatacatgcatcgattctacatgattcagtccttcacacagttccctggaaattctgtggctccagcagccttgtttctagcagctaaagtggaggagcagcccaaaaaattggaacatgtcatcaaggtagcacatacttgtctccatcctcaggaatcccttcctgatactagaagtgaggcttatttgcaacaagttcaagatctggtcattttagaaagcataattttgcagactttaggctttgaactaacaattgatcacccacatactcatgtagtaaagtgcactcaacttgttcgagcaagcaaggacttagcacagacttcttacttcatggcaaccaacagcctgcatttgaccacatttagcctgcagtacacacctcctgtggtggcctgtgtctgcattcacctggcttgcaagtggtccaattgggagatcccagtctcaactgacgggaagcactggtgggagtatgttgacgccactgtgaccttggaacttttagatgaactgacacatgagtttctacagattttggagaaaactcccaacaggctcaaacgcatttggaattggagggcatgcgaggctgccaagaaaacaaaagcagatgaccgaggaacagatgaaaagacttcagagcagacaatcctcaatatgatttcccagagctcttcagacacaaccattgcaggtttaatgagcatgtcaacttctaccacaagtgcagtgccttccctgccagtctccgaagagtcatccagcaacttaaccagtgtggagatgttgccgggcaagcgttggctgtcctcccaaccttctttcaaactagaacctactcagggtcatcggactagtgagaatttagcacttacaggagttgatcattccttaccacaggatggttcaaatgcatttatttcccagaagcagaatagtaagagtgtgccatcagctaaagtgtcactgaaagaataccgcgcgaagcatgcagaagaattggctgcccagaagaggcaactggagaacatggaagccaatgtgaagtcacaatatgcatatgctgcccagaatctcctttctcatcatgatagccattcttcagtcattctaaaaatgcccatagagggttcagaaaaccccgagcggccttttctggaaaaggctgacaaaacagctctcaaaatgagaatcccagtggcaggtggagataaagctgcgtcttcaaaaccagaggagataaaaatgcgcataaaagtccatgctgcagctgataagcacaattctgtagaggacagtgttacaaagagccgagagcacaaagaaaagcacaagactcacccatctaatcatcatcatcatcataatcaccactcacacaagcactctcattcccaacttccagttggtactgggaacaaacgtcctggtgatccaaaacatagtagccagacaagcaacttagcacataaaacctatagcttgtctagttctttttcctcttccagttctactcgtaaaaggggaccctctgaagagactggaggggctgtgtttgatcatccagccaagattgccaagagtactaaatcctcttccctaaatttctccttcccttcacttcctacaatgggtcagatgcctgggcatagctcagacacaagtggcctttccttttcacagcccagctgtaaaactcgtgtccctcattcgaaactggataaagggcccactggggccaatggtcacaacacgacccagacaatagactatcaagacactgtgaatatgcttcactccctgctcagtgcccagggtgttcagcccactcagcccactgcatttgaatttgttcgtccttatagtgactatctgaatcctcggtctggtggaatctcctcgagatctggcaatacagacaaaccccggccaccacctctgccatcagaacctcctccaccacttccaccccttcctaagtaaaaaaagaaaaagaagaggagaaaaaaacttctttaaaaaaacacataatttttctttttttttt

SEQ ID NO: 10 - protein, 726 aa, Homo sapiens
MEGERKNNNKRWYFTREQLENSPSRRFGVDPDKELSYRQQAANLLQDMGQRLNVSQLTINTAIVYMHRFYMIQSFTQFPGNSVAPAALFLAAKVEEQPKKLEHVIKVAHTCLHPQESLPDTRSEAYLQQVQDLVILESIILQTLGFELTIDHPHTHVVKCTQLVRASKDLAQTSYFMATNSLHLTTFSLQYTPPVVACVCIHLACKWSNWEIPVSTDGKHWWEYVDATVTLELLDELTHEFLQILEKTPNRLKRIWNWRACEAAKKTKADDRGTDEKTSEQTILNMISQSSSDTTIAGLMSMSTSTTSAVPSLPVSEESSSNLTSVEMLPGKRWLSSQPSFKLEPTQGHRTSENLALTGVDHSLPQDGSNAFISQKQNSKSVPSAKVSLKEYRAKHAEELAAQKRQLENMEANVKSQYAYAAQNLLSHHDSHSSVILKMPIEGSENPERPFLEKADKTALKMRIPVAGGDKAASSKPEEIKMRIKVHAAADKHNSVEDSVTKSREHKEKHKTHPSNHHHHHNHHSHKHSHSQLPVGTGNKRPGDPKHSSQTSNLAHKTYSLSSSFSSSSSTRKRGPSEETGGAVFDHPAKIAKSTKSSSLNFSFPSLPTMGQMPGHSSDTSGLSFSQPSCKTRVPHSKLDKGPTGANGHNTTQTIDYQDTVNMLHSLLSAQGVQPTQPTAFEFVRPYSDYLNPRSGGISSRSGNTDKPRPPPLPSEPPPPLPPLPK

SEQ ID NO: 11 - DNA, 8089 bp, Homo sapiens
gcggcgcggaggctggcccgggacgcgcccggagcccagggaaggagggaggaggggagggtcgcggccggccgccatggggccgggggcccgtggccgccgccgccgccgtcgcccgatgtcgccgccaccgccaccgccacccgtgcgggcgctgcccctgctgctgctgctagcggggccgggggctgcagcccccccttgcctggacggaagcccgtgtgcaaatggaggtcgttgcacccagctgccctcccgggaggctgcctgcctgtgcccgcctggctgggtgggtgagcggtgtcagctggaggacccctgtcactcaggcccctgtgctggccgtggtgtctgccagagttcagtggtggctggcaccgcccgattctcatgccggtgcccccgtggcttccgaggccctgactgctccctgccagatccctgcctcagcagcccttgtgcccacggtgcccgctgctcagtggggcccgatggacgcttcctctgctcctgcccacctggctaccagggccgcagctgccgaagcgacgtggatgagtgccgggtgggtgagccctgccgccatggtggcacctgcctcaacacacctggctccttccgctgccagtgtccagctggctacacagggccactatgtgagaaccccgcggtgccctgtgcaccctcaccatgccgtaacgggggcacctgcaggcagagtggcgacctcacttacgactgtgcctgtcttcctgggtttgagggtcagaattgtgaagtgaacgtggacgactgtccaggacaccgatgtctcaatggggggacatgcgtggatggcgtcaacacctataactgccagtgccctcctgagtggacaggccagttctgcacggaggacgtggatgagtgtcagctgcagcccaacgcctgccacaatgggggtacctgcttcaacacgctgggtggccacagctgcgtgtgtgtcaatggctggacaggcgagagctgcagtcagaatatcgatgactgtgccacagccgtgtgcttccatggggccacctgccatgaccgcgtggcttctttctactgtgcctgccccatgggcaagactggcctcctgtgtcacctggatgacgcctgtgtcagcaacccctgccacgaggatgctatctgtgacacaaatccggtgaacggccgggccatttgcacctgtcctcccggcttcacgggtggggcatgtgaccaggatgtggacgagtgctctatcggcgccaacccctgcgagcacttgggcaggtgcgtgaacacgcagggctccttcctgtgccagtgcggtcgtggctacactggacctcgctgtgagaccgatgtcaacgagtgtctgtcggggccctgccgaaaccaggccacgtgcctcgaccgcataggccagttcacctgtatctgtatggcaggcttcacaggaacctattgcgaggtggacattgacgagtgtcagagtagcccctgtgtcaacggtggggtctgcaaggaccgagtcaatggcttcagctgcacctgcccctcgggcttcagcggctccacgtgtcagctggacgtggacgaatgcgccagcacgccctgcaggaatggcgccaaatgcgtggaccagcccgatggctacgagtgccgctgtgccgagggctttgagggcacgctgtgtgatcgcaacgtggacgactgctcccctgacccatgccaccatggtcgctgcgtggatggcatcgccagcttctcatgtgcctgtgctcctggctacacgggcacacgctgcgagagccaggtggacgaatgccgcagccagccctgccgccatggcggcaaatgcctagacctggtggacaagtacctctgccgctgcccttctgggaccacaggtgtgaactgcgaagtgaacattgacgactgtgccagcaacccctgcacctttggagtctgccgtgatggcatcaaccgctacgactgtgtctgccaacctggcttcacagggcccctttgtaacgtggagatcaatgagtgtgcttccagcccatgcggcgagggaggttcctgtgtggatggggaaaatggcttccgctgcctctgcccgcctggctccttgcccccactctgcctccccccgagccatccctgtgcccatgagccctgcagtcacggcatctgctatgatgcacctggcgggttccgctgtgtgtgtgagcctggctggagtggcccccgctgcagccagagcctggcccgagacgcctgtgagtcccagccgtgcagggccggtgggacatgcagcagcgatggaatgggtttccactgcacctgcccgcctggtgtccagggacgtcagtgtgaactcctctccccctgcaccccgaacccctgtgagcatgggggccgctgcgagtctgcccctggccagctgcctgtctgctcctgcccccagggctggcaaggcccacgatgccagcaggatgtggacgagtgtgctggccccgcaccctgtggccctcatggtatctgcaccaacctggcagggagtttcagctgcacctgccatggagggtacactggcccttcctgcgatcaggacatcaatgactgtgaccccaacccatgcctgaacggtggctcgtgccaagacggcgtgggctccttttcctgctcctgcctccctggtttcgccggcccacgatgcgcccgcgatgtggatgagtgcctgagcaacccctgcggcccgggcacctgtaccgaccacgtggcctccttcacctgcacctgcccgccaggctacggaggcttccactgcgaacaggacctgcccgactgcagccccagctcctgcttcaatggcgggacctgtgtggacggcgtgaactcgttcagctgcctgtgccgtcccggctacacaggagcccactgccaacatgaggcagacccctgcctctcgcggccctgcctacacgggggcgtctgcagcgccgcccaccctggcttccgctgcacctgcctcgagagcttcacgggcccgcagtgccagacgctggtggattggtgcagccgccagccttgtcaaaacgggggtcgctgcgtccagactggggcctattgcctttgtccccctggatggagcggacgcctctgtgacatccgaagcttgccctgcagggaggccgcagcccagatcggggtgcggctggagcagctgtgtcaggcgggtgggcagtgtgtggatgaagacagctcccactactgcgtgtgcccagagggccgtactggtagccactgtgagcaggaggtggacccctgcttggcccagccctgccagcatggggggacctgccgtggctatatggggggctacatgtgtgagtgtcttcctggctacaatggtgataactgtgaggacgacgtggacgagtgtgcctcccagccctgccagcacgggggttcatgcattgacctcgtggcccgctatctctgctcctgtcccccaggaacgctgggggtgctctgcgagattaatgaggatgactgcggcccaggcccaccgctggactcagggccccggtgcctacacaatggcacctgcgtggacctggtgggtggtttccgctgcacctgtcccccaggatacactggtttgcgctgcgaggcagacatcaatgagtgtcgctcaggtgcctgccacgcggcacacacccgggactgcctgcaggacccaggcggaggtttccgttgcctttgtcatgctggcttctcaggtcctcgctgtcagactgtcctgtctccctgcgagtcccagccatgccagcatggaggccagtgccgtcctagcccgggtcctgggggtgggctgaccttcacctgtcactgtgcccagccgttctggggtccgcgttgcgagcgggtggcgcgctcctgccgggagctgcagtgcccggtgggcgtcccatgccagcagacgccccgcgggccgcgctgcgcctgccccccagggttgtcgggaccctcctgccgcagcttcccggggtcgccgccgggggccagcaacgccagctgcgcggccgccccctgtctccacgggggctcctgccgccccgcgccgctcgcgcccttcttccgctgcgcttgcgcgcagggctggaccgggccgcgctgcgaggcgcccgccgcggcacccgaggtctcggaggagccgcggtgcccgcgcgccgcctgccaggccaagcgcggggaccagcgctgcgaccgcgagtgcaacagcccaggctgcggctgggacggcggcgactgctcgctgagcgtgggcgacccctggcggcaatgcgaggcgctgcagtgctggcgcctcttcaacaacagccgctgcgaccccgcctgcagctcgcccgcctgcctctacgacaacttcgactgccacgccggtggccgcgagcgcacttgcaacccggtgtacgagaagtactgcgccgaccactttgccgacggccgctgcgaccagggctgcaacacggaggagtgcggctgggatgggctggattgtgccagcgaggtgccggccctgctggcccgcggcgtgctggtgctcacagtgctgctgccgccagaggagctactgcgttccagcgccgactttctgcagcggctcagcgccatcctgcgcacctcgctgcgcttccgcctggacgcgcacggccaggccatggtcttcccttaccaccggcctagtcctggctccgaaccccgggcccgtcgggagctggcccccgaggtgatcggctcggtagtaatgctggagattgacaaccggctctgcctgcagtcgcctgagaatgatcactgcttccccgatgcccagagcgccgctgactacctgggagcgttgtcagcggtggagcgcctggacttcccgtacccactgcgggacgtgcggggggagccgctggagcctccagaacccagcgtcccgctgctgccactgctagtggcgggcgctgtcttgctgctggtcattctcgtcctgggtgtcatggtggcccggcgcaagcgcgagcacagcaccctctggttccctgagggcttctcactgcacaaggacgtggcctctggtcacaagggccggcgggaacccgtgggccaggacgcgctgggcatgaagaacatggccaagggtgagagcctgatgggggaggtggccacagactggatggacacagagtgcccagaggccaagcggctaaaggtagaggagccaggcatgggggctgaggaggctgtggattgccgtcagtggactcaacaccatctggttgctgctgacatccgcgtggcaccagccatggcactgacaccaccacagggcgacgcagatgctgatggcatggatgtcaatgtgcgtggcccagatggcttcaccccgctaatgctggcttccttctgtgggggggctctggagccaatgccaactgaagaggatgaggcagatgacacatcagctagcatcatctccgacctgatctgccagggggctcagcttggggcacggactgaccgtactggcgagactgctttgcacctggctgcccgttatgcccgtgctgatgcagccaagcggctgctggatgctggggcagacaccaatgcccaggaccactcaggccgcactcccctgcacacagctgtcacagccgatgcccagggtgtcttccagattctcatccgaaaccgctctacagacttggatgcccgcatggcagatggctcaacggcactgatcctggcggcccgcctggcagtagagggcatggtggaagagctcatcgccagccatgctgatgtcaatgctgtggatgagcttgggaaatcagccttacactgggctgcggctgtgaacaacgtggaagccactttggccctgctcaaaaatggagccaataaggacatgcaggatagcaaggaggagacccccctattcctggccgcccgcgagggcagctatgaggctgccaagctgctgttggaccactttgccaaccgtgagatcaccgaccacctggacaggctgccgcgggacgtagcccaggagagactgcaccaggacatcgtgcgcttgctggatcaacccagtgggccccgcagcccccccggtccccacggcctggggcctctgctctgtcctccaggggccttcctccctggcctcaaagcggcacagtcggggtccaagaagagcaggaggccccccgggaaggcggggctggggccgcaggggccccgggggcggggcaagaagctgacgctggcctgcccgggccccctggctgacagctcggtcacgctgtcgcccgtggactcgctggactccccgcggcctttcggtgggccccctgcttcccctggtggcttcccccttgaggggccctatgcagctgccactgccactgcagtgtctctggcacagcttggtggcccaggccgggcgggtctagggcgccagccccctggaggatgtgtactcagcctgggcctgctgaaccctgtggctgtgcccctcgattgggcccggctgcccccacctgcccctccaggcccctcgttcctgctgccactggcgccgggaccccagctgctcaacccagggacccccgtctccccgcaggagcggcccccgccttacctggcagtcccaggacatggcgaggagtacccggcggctggggcacacagcagccccccaaaggcccgcttcctgcgggttcccagtgagcacccttacctgaccccatcccccgaatcccctgagcactgggccagcccctcacctccctccctctcagactggtccgaatccacgcctagcccagccactgccactggggccatggccaccaccactggggcactgcctgcccagccacttcccttgtctgttcccagctcccttgctcaggcccagacccagctggggccccagccggaagttacccccaagaggcaagtgttggcctgagacgctcgtcagttcttagatcttgggggcctaaagagacccccgtcctgcctcctttctttctctgtctcttccttccttttagtctttttcatcctcttctctttccaccaaccctcctgcatccttgccttgcagcgtgaccgagataggtcatcagcccagggcttcagtcttcctttatttataatgggtgggggctaccacccaccctctcagtcttgtgaagagtctgggacctccttcttccccacttctctcttccctcattcctttctctctccttctggcctctcatttccttacactctgacatgaatgaattattattatttttatttttctttttttttttacattttgtatagaaacaaattcatttaaacaaacttattattattattttttacaaaatatatatatggagatgctccctccccctgtgaaccccccagtgcccccgtggggctgagtctgtgggcccattcggccaagctggattctgtgtacctagtacacaggcatgactgggatcccgtgtaccgagtacacgacccaggtatgtaccaagtaggcacccttgggcgcacccactggggccaggggtcgggggagtgttgggagcctcctccccaccccacctccctcacttcactgcattccagatgggacatgttccatagccttgctggggaagggcccactgccaactccctctgccccagccccacccttggccatctccctttgggaactagggggctgctggtgggaaatgggagccagggcagatgtatgcattcctttgtgtccctgtaaatgtgggactacaagaagaggagctgcctgagtggtactttctcttcctggtaatcctctggcccagcctcatggcagaatagaggtatttttaggctatttttgtaatatggcttctggtcaaaatccctgtgtagctgaattcccaagccctgcattgtacagccccccactcccctcaccacctaataaaggaatagttaacactcaaaaaaaaaaaaaaaaaaa

SEQ ID NO: 12 - protein, 2321 aa, Homo sapiens
MGPGARGRRRRRRPMSPPPPPPPVRALPLLLLLAGPGAAAPPCLDGSPCANGGRCTQLPSREAACLCPPGWVGERCQLEDPCHSGPCAGRGVCQSSVVAGTARFSCRCPRGFRGPDCSLPDPCLSSPCAHGARCSVGPDGRFLCSCPPGYQGRSCRSDVDECRVGEPCRHGGTCLNTPGSFRCQCPAGYTGPLCENPAVPCAPSPCRNGGTCRQSGDLTYDCACLPGFEGQNCEVNVDDCPGHRCLNGGTCVDGVNTYNCQCPPEWTGQFCTEDVDECQLQPNACHNGGTCFNTLGGHSCVCVNGWTGESCSQNIDDCATAVCFHGATCHDRVASFYCACPMGKTGLLCHLDDACVSNPCHEDAICDTNPVNGRAICTCPPGFTGGACDQDVDECSIGANPCEHLGRCVNTQGSFLCQCGRGYTGPRCETDVNECLSGPCRNQATCLDRIGQFTCICMAGFTGTYCEVDIDECQSSPCVNGGVCKDRVNGFSCTCPSGFSGSTCQLDVDECASTPCRNGAKCVDQPDGYECRCAEGFEGTLCDRNVDDCSPDPCHHGRCVDGIASFSCACAPGYTGTRCESQVDECRSQPCRHGGKCLDLVDKYLCRCPSGTTGVNCEVNIDDCASNPCTFGVCRDGINRYDCVCQPGFTGPLCNVEINECASSPCGEGGSCVDGENGFRCLCPPGSLPPLCLPPSHPCAHEPCSHGICYDAPGGFRCVCEPGWSGPRCSQSLARDACESQPCRAGGTCSSDGMGFHCTCPPGVQGRQCELLSPCTPNPCEHGGRCESAPGQLPVCSCPQGWQGPRCQQDVDECAGPAPCGPHGICTNLAGSFSCTCHGGYTGPSCDQDINDCDPNPCLNGGSCQDGVGSFSCSCLPGFAGPRCARDVDECLSNPCGPGTCTDHVASFTCTCPPGYGGFHCEQDLPDCSPSSCFNGGTCVDGVNSFSCLCRPGYTGAHCQHEADPCLSRPCLHGGVCSAAHPGFRCTCLESFTGPQCQTLVDWCSRQPCQNGGRCVQTGAYCLCPPGWSGRLCDIRSLPCREAAAQIGVRLEQLCQAGGQCVDEDSSHYCVCPEGRTGSHCEQEVDPCLAQPCQHGGTCRGYMGGYMCECLPGYNGDNCEDDVDECASQPCQHGGSCIDLVARYLCSCPPGTLGVLCEINEDDCGPGPPLDSGPRCLHNGTCVDLVGGFRCTCPPGYTGLRCEADINECRSGACHAAHTRDCLQDPGGGFRCLCHAGFSGPRCQTVLSPCESQPCQHGGQCRPSPGPGGGLTFTCHCAQPFWGPRCERVARSCRELQCPVGVPCQQTPRGPRCACPPGLSGPSCRSFPGSPPGASNASCAAAPCLHGGSCRPAPLAPFFRCACAQGWTGPRCEAPAAAPEVSEEPRCPRAACQAKRGDQRCDRECNSPGCGWDGGDCSLSVGDPWRQCEALQCWRLFNNSRCDPACSSPACLYDNFDCHAGGRERTCNPVYEKYCADHFADGRCDQGCNTEECGWDGLDCASEVPALLARGVLVLTVLLPPEELLRSSADFLQRLSAILRTSLRFRLDAHGQAMVFPYHRPSPGSEPRARRELAPEVIGSVVMLEIDNRLCLQSPENDHCFPDAQSAADYLGALSAVERLDFPYPLRDVRGEPLEPPEPSVPLLPLLVAGAVLLLVILVLGVMVARRKREHSTLWFPEGFSLHKDVASGHKGRREPVGQDALGMKNMAKGESLMGEVATDWMDTECPEAKRLKVEEPGMGAEEAVDCRQWTQHHLVAADIRVAPAMALTPPQGDADADGMDVNVRGPDGFTPLMLASFCGGALEPMPTEEDEADDTSASIISDLICQGAQLGARTDRTGETALHLAARYARADAAKRLLDAGADTNAQDHSGRTPLHTAVTADAQGVFQILIRNRSTDLDARMADGSTALILAARLAVEGMVEELIASHADVNAVDELGKSALHWAAAVNNVEATLALLKNGANKDMQDSKEETPLFLAAREGSYEAAKLLLDHFANREITDHLDRLPRDVAQERLHQDIVRLLDQPSGPRSPPGPHGLGPLLCPPGAFLPGLKAAQSGSKKSRRPPGKAGLGPQGPRGRGKKLTLACPGPLADSSVTLSPVDSLDSPRPFGGPPASPGGFPLEGPYAAATATAVSLAQLGGPGRAGLGRQPPGGCVLSLGLLNPVAVPLDWARLPPPAPPGPSFLLPLAPGPQLLNPGTPVSPQERPPPYLAVPGHGEEYPAAGAHSSPPKARFLRVPSEHPYLTPSPESPEHWASPSPPSLSDWSESTPSPATATGAMATTTGALPAQPLPLSVPSSLAQAQTQLGPQPEVTPKRQVLA